(19) United States
(12) Patent Application Publication
(10) Pub. No.: US 2025/0312445 A1
(43) Pub. Date: Oct. 9, 2025

(54) METHODS FOR TREATING BLOOD LOSS CONDITIONS

(71) Applicant: Disc Medicine, Inc., Watertown, MA (US)

(72) Inventors: Akshay Buch, West Chester, OH (US); Natasha Novikov, Cambridge, MA (US); Will Savage, Newton Highlands, MA (US); Hua Yang, Acton, MA (US)

(73) Assignee: Disc Medicine, Inc., Watertown, MA (US)

(21) Appl. No.: 18/872,447

(22) PCT Filed: Jun. 8, 2023
(86) PCT No.: PCT/US2023/068107

§ 371 Date: Dec. 6, 2024

Related U.S. Application Data
(60) Provisional application No. 63/350,766, filed on Jun. 9, 2022.

(51) Int. Cl. A61K 39/395; A61K 33/26; A61K 38/17; A61K 38/18; C07K 16/28

(52) U.S. Cl. CPC A61K 39/3955; A61K 33/26; A61K 38/179; A61K 38/1816; C07K 16/28

(57) ABSTRACT

Aspects of the disclosure provides composition and methods for treating a subject having a blood loss condition, the method comprising administering to the subject a hemojuvelin (HIV) antagonist (e.g., anti-HJV antibody).

### RELATED APPLICATIONS

This application claims benefit of U.S. Provisional Application No. 63/350,766, filed Jun. 9, 2022, the entire contents of which are incorporated by reference.

### REFERENCE TO AN ELECTRONIC SEQUENCE LISTING

The contents of the electronic sequence listing (D084270007WO00-SEQ-LJG.xml; Size: 202,362 bytes; and Date of Creation: Jun. 7, 2023) is herein incorporated by reference in its entirety.

### BACKGROUND

Iron is a key component of oxygen-transporting storage molecules, such as hemoglobin and myoglobin. Blood loss conditions cause iron deficiency. Hepcidin is a key peptide hormonal regulator of systemic iron homeostasis. It exerts its regulatory function by binding to the cellular iron exporter ferroportin, a transmembrane protein present on hepatocytes, enterocytes in the duodenum, macrophages, and adipocytes. The binding of hepcidin promotes ferroportin degradation, preventing the export of iron from cells and release of iron into the plasma.

### SUMMARY

Aspects of the present disclosure relate to compositions and methods for treating subjects having a blood loss condition. In some embodiments, the treatment described herein promotes hematological recovery such as recovery of erythropoiesis, reticulocyte hemoglobin (CHr), corpuscular hemoglobin (MCH) and/or circulating hemoglobin levels to baseline levels.

In some aspects, the present disclosure provides a method for treating a subject having a blood loss condition, the method comprising administering to the subject a hemojuvelin antagonist.

In some embodiments, the hemojuvelin antagonist is administered in an amount effective for promoting hematological recovery. In some embodiments, the hematological recovery comprises recovery of erythropoiesis, reticulocyte hemoglobin content (CHr), mean corpuscular hemoglobin (MCH) and/or circulating hemoglobin levels to baseline levels prior to the blood loss condition. In some embodiments, the hematological recovery is achieved within a shorter duration than would be achieved by a control subject who did not receive the hemojuvelin antagonist. In some embodiments, the hematological recovery comprises recovery of erythropoiesis, reticulocyte hemoglobin (CHr), mean corpuscular hemoglobin (MCH) and/or circulating hemoglobin levels to baseline levels within 3 days, 5 days, 1 week, two weeks, three weeks, or four weeks.

In some embodiments, the blood loss condition comprises chronic blood loss. In some embodiments, the blood loss condition comprises acute blood loss.

In some embodiments, the blood loss condition comprises iatrogenic blood loss. In some embodiments, the blood loss condition comprises a phlebotomy procedure. In some embodiments, the blood loss condition comprises a surgical procedure. In some embodiments, the blood loss condition comprises a blood donation procedure.

In some embodiments, the blood loss condition comprises a bleeding wound. In some embodiments, the bleeding wound is an internal bleeding wound. In some embodiments, the internal bleeding wound is selected from ruptured blood vessels, organ contusion, organ rupture, and hematoma. In some embodiments, the bleeding wound is an external bleeding wound. In some embodiments, the external bleeding wound is selected from a cut, a stab, a puncture, an avulsion, an incision, and a penetration.

In some embodiments, the blood loss condition comprises a disease.

In some embodiments, the disease is a gastrointestinal (GI) disease. In some embodiments, the GI disease comprises blood loss due to esophageal varices, gastritis, gastric ulcer, duodenal ulcer, diverticulosis, Meckel's diverticulum, intestinal polyps, inflammatory bowel disease (IBD), hemorrhoids, celiac disease or colorectal cancer.

In some embodiments, the disease a genitourinary disease. In some embodiments, the genitourinary disease comprises blood loss due to menorrhagia, fibroid, endometriosis, bladder tumors, urinary tract infection (UTI) or renal stones.

In some embodiments, the disease is an infectious disease. In some embodiments, infectious disease comprises blood loss due to a viral hemorrhagic fever selected from Dengue fever, Ebola virus disease, Lassa fever, Hantavirus pulmonary syndrome, Marburg virus disease, and yellow fever. In some embodiments, the infectious disease comprises blood loss due to a bacterial infectious disease selected from sepsis, bacterial vaginosis, Lemierre's syndrome, and tuberculosis. In some embodiments, the infectious disease comprises blood loss due to a parasitic infectious disease selected from malaria, Trichuriasis, and Schistosomiasis.

In some embodiments, prior to administration, the subject has a hemoglobin level of at least 6 g/dl. In some embodiments, prior to administration, the subject has a hemoglobin level in the range of 7-12 g/dl. In some embodiments, prior to administration, the subject has a hemoglobin level in the range of 7-11 g/dl. In some embodiments, prior to administration, the subject has a hemoglobin level in the range of 7-10 g/dl. In some embodiments, after administration, the subject's hemoglobin level increases by 1-3 g/dl. In some embodiments, after administration, the subject's hemoglobin level increases by 1-3 g/dl within 1 week, two weeks, three weeks, four weeks, five weeks, or six weeks.

In some embodiments, prior to administration, the subject has a ferritin level in the range of up to 5000 ng/ml. In some embodiments, prior to administration, the subject has a ferritin level in the range of 14-150 ng/ml. In some embodiments, after administration, the subject's ferritin level decreases by 15%-50% compared to the ferritin level prior to administration.

In some embodiments, prior to administration, the subject has a TSAT % level in the range of 15%-30%. In some embodiments, after administration, the subject has a TSAT % level in the range of 30%-50%. In some embodiments, the subject has a TSAT % level in the range of 30%-50% within 1 week, two weeks, three weeks, four weeks, five weeks, or six weeks.

In some embodiments, prior to administration, the subject has a reticulocyte hemoglobin (CHr) level in the range of 20-40 pg. In some embodiments, after administration, the subject's CHr level increases by 1%-5% compared to the CHr level prior to administration. In some embodiments, the subject's CHr level increases by 1%-5% within 1 day, 2 days, 3 days, 4 days, 5 days, 6 days, one week, or two weeks.

In some embodiments, prior to administration, the subject has a hepcidin level in the range of 5-75 ng/ml. In some embodiments, after administration, the subject's hepcidin level decreases by 2-60 ng/ml compared to the hepcidin level prior to administration.

In some embodiments, prior to administration, the subject has a red blood cell count in the range of 3×10¹² to 6×10¹² cells/L. In some embodiments, after administration, the subject's red blood cell count increases by at least 0.5×10¹² cells/L compared to the red blood cell count prior to administration.

In some embodiments, prior to administration, the subject has a MCH level of 15-50 pg. In some embodiments, after administration, the subject's MCH level increases by 1%-5% compared to the MCH level prior to administration.

In some embodiments, the subject does not have a functional iron deficiency prior to administration.

In some embodiments, the subject does not have anemia associated with inflammation prior to administration. In some embodiments, the subject has anemia associated with inflammation prior to administration.

In some embodiments, the blood loss condition comprises persistent blood loss. In some embodiments, the blood loss condition comprises more than one intermittent blood loss instance up to one week apart, up to two weeks apart, or up to one month apart. In some embodiments, the blood loss condition comprises loss of up to 10% of the subject's total blood volume. In some embodiments, the blood loss is in the range of 1-10% of the subject's total blood volume. In some embodiments, the blood loss condition comprises persistent blood loss which occurs within 1 hour, 4 hours, 8 hours, 12 hours, 16 hours, 20 hours, 1 day, 3 days, 5 days, 8 days, 10 days, two weeks, three weeks, one month, two months, or three months. In some embodiments, the blood loss condition comprises persistent blood loss which persists at least 1 hour, at least 4 hours, at least 8 hours, at least 12 hours, at least 16 hours, at least 20 hours, at least 1 day, at least 3 days, at least 5 days, at least 8 days, at least 10 days, at least two weeks, at least three weeks, at least one month, at least two months, or at least three months. In some embodiments, the blood loss condition comprises intermittent blood loss instance wherein each instance of blood loss comprises loss of up to 10% of the subject's total blood volume. In some embodiments, each instance of blood loss occurs up to 1 hour, up to 4 hours, up to 8 hours, up to 12 hours, up to 16 hours, up to 20 hours, up to 1 day, up to 3 days, up to 5 days, up to 8 days, up to 10 days, or up to two weeks apart.

In some embodiments, the subject has a ferritin level of 14-80 ng/ml due to the blood loss. In some embodiments, the subject has a serum iron of at least 40 μg/dL.

In some embodiments, the hemojuvelin antagonist is an anti-hemojuvelin (HJV) antibody, e.g., the anti-HJV antibodies described herein.

In some embodiments, the hemojuvelin antagonist is administered to the subject weekly, twice a month, or once a month.

In some embodiments, the subject is not identified as requiring blood transfusion. In some embodiments, the subject is identified as requiring blood transfusion prior to administration of the hemojuvelin antagonist.

In some embodiments, the hemojuvelin antagonist is administered in combination with oral iron supplement or iron injection.

In some embodiments, the hemojuvelin antagonist is administered while the subject is on a gluten-free diet.

In some embodiments, the hemojuvelin antagonist is administered in combination with a therapeutic agent. In some embodiments, the therapeutic agent is EPO. In some embodiments, the therapeutic agent is Luspatercept. In some embodiments, the therapeutic agent is HIF-PHI. In some embodiments, the therapeutic agent is for treating the disease that is associated with blood loss.

In some embodiments, the hemojuvelin antagonist is administered subcutaneously. In some embodiments, the hemojuvelin antagonist is administered intravenously.

In some embodiments, the subject is administered the hemojuvelin antagonist on multiple occasions. In some embodiments, the occasions are on a monthly interval.

Other aspects of the disclosure relate to a method of treating a subject having anemia, the method comprising administering to the subject an effective amount of anti-hemojuvelin antibody in monthly interval. In some embodiments, the anemia is associated with a functional iron deficiency. In some embodiments, the anemia is associate with inflammation.

In some embodiments, the anti-hemojuvelin antibody is administered subcutaneously. In some embodiments, the anti-hemojuvelin antibody is administered intravenously.

In some embodiments, the anti-hemojuvelin antibody is administered at a dose of between 25 mg and 70 mg. In some embodiments, the anti-hemojuvelin antibody is administered at a dose of between 35 mg and 65 mg. In some embodiments, the anti-hemojuvelin antibody is administered at a dose of between 45 mg and 60 mg. In some embodiments, the anti-hemojuvelin antibody is administered at a dose of 56 mg.

The foregoing and other aspects, implementations, acts, functionalities, features, and embodiments of the present teachings can be more fully understood from the following description in conjunction with the accompanying drawings.

In some embodiments, a subject in need thereof (e.g., a subject having a blood loss condition described herein) is administered an anti-HJV antibody comprising: a HC CDR1 comprising the amino acid sequence selected from SEQ ID NOs: 1 or 9; a HC CDR2 comprising the amino acid sequence selected from SEQ ID NOs: 2 or 10; a HC CDR3 comprising the amino acid sequence selected from SEQ ID NOs: 3, 11, 12, or 13; a LC CDR1 comprising the amino acid sequence selected from SEQ ID NOs: 4, 14, 15, 16, 17, 50; a LC CDR2 comprising the amino acid sequence selected from SEQ ID NOs: 5, 49, 18, 19, 20, 21, 22, or 23; and a LC CDR3 comprising the amino acid sequence selected from SEQ ID NOs: 6, 24, 25, 26, 27, 28, or 29. In some embodiments, a subject in need thereof (e.g., a subject having a blood loss condition described herein) is administered an anti-HJV antibody comprising a VH comprising the amino acid sequence selected from SEQ ID NOs: 7, 34, 36, 38, 42, or 44; and a VL comprising the amino acid sequence selected from SEQ ID NOs: 8, 30, 31, 32, 33, 35, 37, 39, 41, 43 or 45. In some embodiments, a subject in need thereof (e.g., a subject having a blood loss condition described herein) is administered an anti-HJV antibody comprising a HC sequence comprising the amino acid selected from SEQ ID NOS: 51, 114, 57, 115, 59, 116, 61, 117, 63, 118, 66, 119, 68, or 120; and a LC comprising the amino acid sequence selected from SEQ ID NOs: 52, 53, 54, 55, 56, 58, 60, 62, 65, 67, or 69.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 17, a LC CDR2 comprising the amino acid sequence set forth in SEQ ID NO: 5, and a LC CDR3 comprising the amino acid sequence set forth in SEQ ID NO: 27. In some embodiments, a subject in need thereof (e.g., a subject having a blood loss condition described herein) is administered an anti-HJV antibody comprising a VH comprising the amino acid sequence set forth in SEQ ID NO: 38, and a VL comprising the amino acid sequence set forth in SEQ ID NO: 39. In some embodiments, a subject in need thereof is administered an anti-HJV antibody comprising a HC comprising the amino acid sequence set forth in SEQ ID NOs: 61 or 117, and a LC comprising the amino acid sequence set forth in SEQ ID NO: 62. In some embodiments, a subject in need thereof (e.g., a subject having a blood loss condition described herein) is administered an anti-HJV antibody comprising a HC comprising the amino acid sequence set forth in SEQ ID NOs: 63 or 118, and a LC comprising the amino acid sequence set forth in SEQ ID NO: 62.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 4, a LC CDR2 comprising the amino acid sequence set forth in SEQ ID NO: 5, and a LC CDR3 comprising the amino acid sequence set forth in SEQ ID NO: 6. In some embodiments, a subject in need thereof (e.g., a subject having a blood loss condition described herein) is administered an anti-HJV antibody comprising a VH comprising the amino acid sequence set forth in SEQ ID NO: 7, and a VL comprising the amino acid sequence set forth in SEQ ID NO: 8. In some embodiments, a subject in need thereof (e.g., a subject having a blood loss condition described herein) is administered an anti-HJV antibody comprising a HC comprising the amino acid sequence set forth in SEQ ID NOs: 51 or 114, and a LC comprising the amino acid sequence set forth in SEQ ID NO: 52.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 4, a LC CDR2 comprising the amino acid sequence set forth in SEQ ID NO: 49, and a LC CDR3 comprising the amino acid sequence set forth in SEQ ID NO: 24. In some embodiments, a subject in need thereof (e.g., a subject having a blood loss condition described herein) is administered an anti-HJV antibody comprising a VH comprising the amino acid sequence set forth in SEQ ID NO: 7, and a VL comprising the amino acid sequence set forth in SEQ ID NO: 30. In some embodiments, a subject in need thereof (e.g., a subject having a blood loss condition described herein) is administered an anti-HJV antibody comprising a HC comprising the amino acid sequence set forth in SEQ ID NOs: 51 or 114, and a LC comprising the amino acid sequence set forth in SEQ ID NO: 53.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 4, a LC CDR2 comprising the amino acid sequence set forth in SEQ ID NO: 18, and a LC CDR3 comprising the amino acid sequence set forth in SEQ ID NO: 25. In some embodiments, a subject need thereof (e.g., a subject having a blood loss condition described herein) is administered an anti-HJV antibody comprising a VH comprising the amino acid sequence set forth in SEQ ID NO: 7, and a VL comprising the amino acid sequence set forth in SEQ ID NO: 31. In some embodiments, a subject in need thereof (e.g., a subject having a blood loss condition described herein) is administered an anti-HJV antibody comprising a HC comprising the amino acid sequence set forth in SEQ ID NOs: 51 or 114, and a LC comprising the amino acid sequence set forth in SEQ ID NO: 54.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 4, a LC CDR2 comprising the amino acid sequence set forth in SEQ ID NO: 49, and a LC CDR3 comprising the amino acid sequence set forth in SEQ ID NO: 24. In some embodiments, a subject in need thereof (e.g., a subject having a blood loss condition described herein) is administered an anti-HJV antibody comprising a VH comprising the amino acid sequence set forth in SEQ ID NO: 7, and a VL comprising the amino acid sequence set forth in SEQ ID NO: 30. In some embodiments, a subject in need thereof (e.g., a subject having a blood loss condition described herein) is administered an anti-HJV antibody comprising a HC comprising the amino acid sequence set forth in SEQ ID NOs: 51 or 114, and a LC comprising the amino acid sequence set forth in SEQ ID NO: 53.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 14, a LC CDR2 comprising the amino acid sequence set forth in SEQ ID NO: 19, and a LC CDR3 comprising the amino acid sequence set forth in SEQ ID NO: 25. In some embodiments, a subject in need thereof (e.g., a subject having a blood loss condition described herein) is administered an anti-HJV antibody comprising a VH comprising the amino acid sequence set forth in SEQ ID NO: 7, and a VL comprising the amino acid sequence set forth in SEQ ID NO: 32. In some embodiments, a subject in need thereof (e.g., a subject having a blood loss condition described herein) is administered an anti-HJV antibody comprising a HC comprising the amino acid sequence set forth in SEQ ID NOs: 51 or 114, and a LC comprising the amino acid sequence set forth in SEQ ID NO: 55.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 15, a LC CDR2 comprising the amino acid sequence set forth in SEQ ID NO: 20, and a LC CDR3 comprising the amino acid sequence set forth in SEQ ID NO: 26. In some embodiments, a subject in need thereof (e.g., a subject having a blood loss condition described herein) is administered an anti-HJV antibody comprising a VH comprising the amino acid sequence set forth in SEQ ID NO: 7, and a VL comprising the amino acid sequence set forth in SEQ ID NO: 33. In some embodiments, a subject in need thereof (e.g., a subject having a blood loss condition described herein) is administered an anti-HJV antibody comprising a HC comprising the amino acid sequence set forth in SEQ ID NOs: 51 or 114, and a LC comprising the amino acid sequence set forth in SEQ ID NO: 56.

In some embodiments, a subject in need thereof (e.g., a subject having a blood loss condition described herein) is administered an anti-HJV antibody comprising: a HC CDR1 comprising the amino acid sequence set forth in SEQ ID NO: 9, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 16, a LC CDR2 comprising the amino acid sequence set forth in SEQ ID NO: 21, and a LC CDR3 comprising the amino acid sequence set forth in SEQ ID NO: 27. In some embodiments, a subject in need thereof (e.g., a subject having a blood loss condition described herein) is administered an anti-HJV antibody comprising a VH comprising the amino acid sequence set forth in SEQ ID NO: 34, and a VL comprising the amino acid sequence set forth in SEQ ID NO: 35. In some embodiments, a subject in need thereof (e.g., a subject having a blood loss condition described herein) is administered an anti-HJV antibody comprising a HC comprising the amino acid sequence set forth in SEQ ID NOs: 57 or 115, and a LC comprising the amino acid sequence set forth in SEQ ID NO: 58.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 10, a HC CDR3 comprising the amino acid sequence set forth in SEQ ID NO: 11, a LC CDR1 comprising the amino acid sequence set forth in SEQ ID NO: 17, a LC CDR2 comprising the amino acid sequence set forth in SEQ ID NO: 18, and a LC CDR3 comprising the amino acid sequence set forth in SEQ ID NO: 28. In some embodiments, a subject in need thereof (e.g., a subject having a blood loss condition described herein) is administered an anti-HJV antibody comprising a VH comprising the amino acid sequence set forth in SEQ ID NO: 36, and a VL comprising the amino acid sequence set forth in SEQ ID NO: 37. In some embodiments, a subject in need thereof (e.g., a subject having a blood loss condition described herein) is administered an anti-HJV antibody comprising a HC comprising the amino acid sequence set forth in SEQ ID NOs: 59 or 116, and a LC comprising the amino acid sequence set forth in SEQ ID NO: 60.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 3, a LC CDR1 comprising the amino acid sequence set forth in SEQ ID NO: 50, a LC CDR2 comprising the amino acid sequence set forth in SEQ ID NO: 22, and a LC CDR3 comprising the amino acid sequence set forth in SEQ ID NO: 28. In some embodiments, a subject in need thereof (e.g., a subject having a blood loss condition described herein) is administered an anti-HJV antibody comprising a VH comprising the amino acid sequence set forth in SEQ ID NO: 38, and a VL comprising the amino acid sequence set forth in SEQ ID NO: 41. In some embodiments, a subject in need thereof is administered an anti-HJV antibody comprising a HC comprising the amino acid sequence set forth in SEQ ID NOs: 61 or 117, and a LC comprising the amino acid sequence set forth in SEQ ID NO: 65.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 12, a LC CDR1 comprising the amino acid sequence set forth in SEQ ID NO: 15, a LC CDR2 comprising the amino acid sequence set forth in SEQ ID NO: 23, and a LC CDR3 comprising the amino acid sequence set forth in SEQ ID NO: 27. In some embodiments, a subject in need thereof (e.g., a subject having a blood loss condition described herein) is administered an anti-HJV antibody comprising a VH comprising the amino acid sequence set forth in SEQ ID NO: 42, and a VL comprising the amino acid sequence set forth in SEQ ID NO: 43. In some embodiments, a subject in need thereof (e.g., a subject having a blood loss condition described herein) is administered an anti-HJV antibody comprising a HC comprising the amino acid sequence set forth in SEQ ID NOs: 66 or 119, and a LC comprising the amino acid sequence set forth in SEQ ID NO: 67.

In some embodiments, a subject in need thereof (e.g., a subject having a blood loss condition described herein) is administered an anti-HJV antibody comprising: a HC CDR1 comprising the amino acid sequence set forth in SEQ ID NO: 1, a HC CDR2 comprising the amino acid sequence set forth in SEQ ID NO: 2, a HC CDR3 comprising the amino acid sequence set forth in SEQ ID NO: 13, a LC CDR1 comprising the amino acid sequence set forth in SEQ ID NO: 16, a LC CDR2 comprising the amino acid sequence set forth in SEQ ID NO: 21, and a LC CDR3 comprising the amino acid sequence set forth in SEQ ID NO: 29. In some embodiments, a subject in need thereof (e.g., a subject having a blood loss condition described herein) is administered an anti-HJV antibody comprising a VH comprising the amino acid sequence set forth in SEQ ID NO: 44, and a VL comprising the amino acid sequence set forth in SEQ ID NO: 45. In some embodiments, a subject in need thereof (e.g., a subject having a blood loss condition described herein) is administered an anti-HJV antibody comprising a HC comprising the amino acid sequence set forth in SEQ ID NOs: 68 or 120, and a LC comprising the amino acid sequence set forth in SEQ ID NO: 69.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 depicts a schematic showing that inhibiting hemojuvelin (HJV) with an HJV antagonist antibody reduces the expression of HAMP, the gene that encodes hepcidin.

FIG. 2 depicts the study design of a single ascending dose of an anti-HJV antibody hHA-008, double-blind, placebo-controlled trial in healthy volunteers.

FIGS. 3A-3C show results of the pharmacokinetic and pharmacodynamic parameters of anti-HJV antibody hHA-008 in humans. FIG. 3A shows hHA-008 concentration-time profiles in human serum plotted as mean with error bars representing standard error of the mean in 7 mg intravenous injection (IV), 14 mg subcutaneous injection (SC), 28 mg SC, and 56 mg SC. FIGS. 3B-3C show the mean absolute change (A) from baseline in hepcidin (FIG. 3B) and transferrin saturation (TSAT %) in human serum following treatment with hHa-008 at the specified concentrations (FIG. 3C). Data are shown for pooled placebo (PBO) (n=8), 7 mg IV (n=8), 14 mg SC (n=6), 28 mg SC (n=6), and 56 mg SC (n=6).

FIGS. 4A-4C depict ferritin and hemoglobin responses to hHA-008 treatment in humans. Mean percent change (4) from baseline of ferritin (FIG. 4A), reticulocyte hemoglobin (CHr) (FIG. 4B) and mean corpuscular hemoglobin (MCH) (FIG. 4C) were measured in serum from volunteers treated with pooled placebo (PBO) (n=8), 7 mg IV (n=8), 14 mg SC (n=6), 28 mg SC (n=6), or 56 mg SC (n=6).

FIGS. 5A-5C depict robust hepcidin, TSAT, and erythropoietic responses in human participants treated with a single dose of 56 mg SC hHA-008. FIG. 5A shows the mean pharmacokinetic (PK) profile (left Y-axis) and pharmacodynamic (PD) response (right Y-axis) measured using hepcidin and transferrin saturation (TSAT) for the 56 mg SC cohort (n=6). FIGS. 5B-5C show the mean with error bars representing standard error of the mean of hemoglobin (FIG. 5B) and red blood cell (RBC) count (FIG. 5C) for pooled placebo (PBO) (n=8) and 56 mg SC (n=6). Asterisk (*) indicates statistically significant, two-sample t-test comparison of Day 42 hemoglobin, assessed as change from baseline.

### DETAILED DESCRIPTION

According to some aspects, the disclosure provides compositions and methods for treating a subject having a blood loss condition, the method comprising administering to the subject a hemojuvelin (HJV) antagonist (e.g., anti-HJV antibody).

Further aspects of the disclosure, including a description of defined terms, are provided below.

### I. Definitions

Administering: As used herein, the terms “administering” or “administration” means to provide a complex to a subject in a manner that is physiologically and/or pharmacologically useful (e.g., to treat a condition in the subject).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a F(ab′)2 fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (a), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or CH3 domain. In some embodiments, the amino acid sequence of the V_{H} domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the V_{H} domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

Affinity Matured Antibody: “Affinity Matured Antibody” is used herein to refer to an antibody with one or more alterations in one or more CDRs, which result in an improvement in the affinity (i.e., KD, kd or ka) of the antibody for a target antigen compared to a parent antibody, which does not possess the alteration(s). Exemplary affinity matured antibodies will have nanomolar or even picomolar affinities for the target antigen. A variety of procedures for producing affinity matured antibodies are known in the art, including the screening of a combinatory antibody library that has been prepared using bio-display. For example, Marks et al., BioTechnology, 10:779-783 (1992) describes affinity maturation by VH and VL domain shuffling. Random mutagenesis of CDR and/or framework residues is described by Barbas et al., Proc. Nat. Acad. Sci. USA, 91:3809-3813 (1994); Schier et al., Gene, 169:147-155 (1995); Yelton et al., J. Immunol., 155:1994-2004 (1995); Jackson et al., J. Immunol., 154 (7): 3310-3319 (1995); and Hawkins et al, J. Mol. Biol., 226:889-896 (1992). Selective mutation at selective mutagenesis positions and at contact or hypermutation positions with an activity-enhancing amino acid residue is described in U.S. Pat. No. 6,914,128 B1.

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Baseline level: As used herein, the term “baseline level” refers to a value that represents the beginning level (including normal or diseased level) of a measurable quality, used for comparison with values representing response to an intervention (e.g., a drug, a medical procedural, or a control). In some embodiments, the baseline reflects the health state—disease severity, confounding conditions, biomarkers (e.g., hematological biomarkers) —present in a person or group of individuals at the beginning of a process. In some embodiments, the process is a medical treatment plan. In some embodiments, the process is a prospective study (e.g., clinical trial). In some embodiments, the baseline value is the last non-missing value before a subject starts the process (e.g., treatment plan or clinical trial). In some embodiments, the baseline value is the value before a subject suffers a condition (e.g., a blood loss condition described herein). In some embodiments, the subject experiences a change in values compared to the baseline value prior to receiving the drug, and treatment using the drug brings the values back to baseline levels. In some embodiments, treatment of a drug brings the values back to baseline levels more rapidly compared to a subject who did not receive the drug.

Blood Loss Condition: As used herein, the term “blood loss condition” refers to a condition (including but not limited to a condition comprising a disease, a disorder, an injury, and/or a procedure, e.g., a medical or surgical procedure) associated with blood loss that creates a need for hematological recovery to achieve homeostatic oxygen carrying capacity in the blood of the subject. In some embodiments, the blood loss can be actual ongoing blood loss. In some embodiments, blood loss may be a past blood loss. In some embodiments, blood loss can be an anticipated blood loss (e.g., before a scheduled surgical procedure or blood donation procedure when blood loss is anticipated to occur). In some embodiments, blood loss can be chronic or acute. In some embodiments, blood loss can be intermittent or persistent.

Comorbidity: As used herein, a “comorbidity” refers to one or more conditions or disorders that co-occur with (or are coincident with) a primary condition (such as a disease associated with a blood loss condition) in an individual.

Control subject: A subject that has comparable features and property as the subject, e.g., age, species, state of health, and other similar parameters. In some embodiments, control subject may be a group of subjects having a similar condition (e.g., a blood loss condition) but is receiving, or expected to receive a different treatment from the therapy described herein (e.g., treatment using an hemojuvelin antagonist). In some embodiments, in an experiment or clinical trial, control subjects may be a group of participants who have characteristics similar to those of the treatment group, but they do not receive the treatment being studied. In some embodiments, the control subjects receive a substance or treatment which is designed to have no therapeutic value (i.e., a placebo).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each V_{H} and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics information system® http://www.imgt.org, Lefranc, M.-P. et al., Nucleic Acids Res., 27:209-212 (1999); Ruiz, M. et al., Nucleic Acids Res., 28:219-221 (2000); Lefranc, M.-P., Nucleic Acids Res., 29:207-209 (2001); Lefranc, M.-P., Nucleic Acids Res., 31:307-310 (2003); Lefranc, M.-P. et al., In Silico Biol., 5, 0006 (2004) [Epub], 5:45-60 (2005); Lefranc, M.-P. et al., Nucleic Acids Res., 33: D593-597 (2005); Lefranc, M.-P. et al., Nucleic Acids Res., 37: D1006-1012 (2009); Lefranc, M.-P. et al., Nucleic Acids Res., 43: D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). ee also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

Generally, there are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although exemplary embodiments use Kabat or Chothia defined CDRs.

CDR-grafted antibody: The term “CDR-grafted antibody” refers to antibodies which comprise heavy and light chain variable region sequences from one species but in which the sequences of one or more of the CDR regions of V_{H} and/or VL are replaced with CDR sequences of another species, such as antibodies having murine heavy and light chain variable regions in which one or more of the murine CDRs (e.g., CDR3) has been replaced with human CDR sequences.

Chimeric antibody: The term “chimeric antibody” refers to antibodies which comprise heavy and light chain variable region sequences from one species and constant region sequences from another species, such as antibodies having murine heavy and light chain variable regions linked to human constant regions.

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Conservative amino acid substitution: As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Fourth Edition, Cold Spring Harbor Laboratory Press, Cold Spring Harbor, New York, 2012, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

Cross-reactive: As used herein and in the context of a targeting agent (e.g., antibody), the term “cross-reactive,” refers to a property of the agent being capable of specifically binding to more than one antigen of a similar type or class (e.g., antigens of multiple homologs, paralogs, or orthologs) with similar affinity or avidity. For example, in some embodiments, an antibody that is cross-reactive against human and non-human primate antigens of a similar type or class (e.g., a human hemojuvelin and non-human primate hemojuvelin) is capable of binding to the human antigen and non-human primate antigens with a similar affinity or avidity. In some embodiments, an antibody is cross-reactive against a human antigen and a rodent antigen of a similar type or class. In some embodiments, an antibody is cross-reactive against a rodent antigen and a non-human primate antigen of a similar type or class. In some embodiments, an antibody is cross-reactive against a human antigen, a non-human primate antigen, and a rodent antigen of a similar type or class.

Effective Amount: As used herein, “an effective amount” or “an amount effective” refers to the amount of each active agent (e.g., hemojuvelin antagonist including an anti-HJV antibody) required to confer therapeutic effect on the subject (such as in treating a blood loss condition associated with, either alone or in combination with one or more other active agents. In some embodiments, the therapeutic effect is reduced hepcidin level or activity, increased level of transferrin saturation (TSAT %), decreased level of circulating transferrin level, decreased ferritin, and/or promote hematological recovery (e.g., promote recovery of erythropoiesis, reticulocyte hemoglobin (CHr), mean corpuscular hemoglobin (MCH) and/or circulating hemoglobin levels).

Erythropoiesis: As used herein, the term “erythropoiesis” refers to the process of producing red blood cells (RBCs or erythrocytes). In some embodiments, erythropoiesis comprises development from hematopoietic stem cells to mature red blood cells. For example, in the process of red blood maturation, a cell generally undergoes a series of differentiation steps, which may include, in the bone marrow, hemocytoblasts (a multipotent hematopoietic stem cells) differentiating along a path comprising one or more of common myeloid progenitors, unipotent stem cells, proerythroblasts, erythroblasts, polychromatophilic cells, and orthochromatic cells. In some embodiments, nuclear expulsion occurs at the end of the orthochromatic stage through an asymmetric division of the orthochromatic erythroblast, becoming a reticulocyte, which is an immature red blood cell. The reticulocytes are generally released from the bone marrow into the circulation, and ultimately become “erythrocytes” or mature red blood cells one or two days later. Therefore, reticulocytes are primarily present in circulating blood. In some embodiments, by the reticulocyte stage, the cell has extruded its nucleus, but is still capable of producing hemoglobin. In further embodiments, normal RBC count in a subject is in the range of 4.7×10¹² to 6.1×10¹² cells/L in men or 4.2×10¹² to 5.4×10¹² cells/L.

Ferritin: As used herein, the term “ferritin” refers to a protein that stores iron and releases it in a controlled fashion. It is the primary intracellular iron-storage protein in both prokaryotes and eukaryotes, keeping iron in a soluble and non-toxic form. In humans, it acts as a buffer against iron deficiency and iron overload. Circulating ferritin is also an indirect marker of the total amount of iron stored in the body; hence, serum ferritin is used as a diagnostic test for iron-deficiency anemia.

Free iron is toxic to cells as it acts as a catalyst in the formation of free radicals from reactive oxygen species via the Fenton reaction. Hence vertebrates have an elaborate set of protective mechanisms to bind iron in various tissue compartments. Ferritin serves to store iron in a non-toxic form, to deposit it in a safe form, and to transport it to areas where it is required. One of the triggers for the production of ferritin is the presence of iron. Under steady-state conditions, the level of ferritin in the blood serum correlates with total body stores of iron; thus, the serum ferritin FR5R1 is the most convenient laboratory test to estimate iron stores. Iron is released from ferritin for use by ferritin degradation, which is performed mainly by lysosomes.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Hemojuvelin (HJV): As used herein, the term “hemojuvelin (HJV)” (also known as repulsive guidance molecule C (RGMc) or hemochromatosis type 2 protein (HFE2)) refers to a membrane-bound and soluble form protein that regulates hepcidin production through the bone morphogenic protein (BMP)/SMAD signaling pathway (Xiao et al., “Bone morphogenic proteins in iron homeostasis.” Bone. 2020; 138:115495). The HFE2 gene encodes two known classes of glycosylphosphatidylinositol (GPI)-anchored and glycosylated HJV molecules, which are targeted to the membrane and undergo distinct fates. HJV exists in multiple isoforms, including two soluble isoforms and two membrane-associated isoforms. In some embodiments, a predominant membrane-associated isoform is a disulfide-linked two-chain form composed of N- and C-terminal fragments. In some embodiments, a full-length single-chain isoform associates with the membrane, but is released from the cell surface and accumulates in extracellular fluid. In some embodiments, HJV may be of human (NCBI Gene ID 148738), non-human primate (e.g., NCBI Gene ID 698805), or rodent (e.g., NCBI Gene ID 69585 or NCBI Gene ID 310681) origin. In addition to HJV (RGMc), the repulsive guidance molecule family includes repulsive guidance molecule A (RGMa) and repulsive guidance molecule B (RGMb). RGMa and RGMb are expressed in the central nervous system during development and are thought to be involved in controlling axonal patterning and neuronal survival, while HJV is produced in the liver and in cardiac and skeletal muscle.

Hepcidin: As used herein, a “hepcidin” refers to an iron-regulating peptide hormone primarily made in the liver that is encoded by the HAMP gene. In some embodiments, hepcidin controls the delivery of iron to blood plasma from intestinal cells absorbing iron, from erythrocyte-recycling macrophages, and from iron-storing hepatocytes. Normal hepcidin levels vary depending on the measurement technique, gender, tissue or fluid in which it is measured, and/or menopausal status. In some embodiments, hepcidin inhibits iron transport by binding to the iron export channel ferroportin which is located on the basolateral surface of gut enterocytes and the plasma membrane of reticuloendothelial cells (macrophages). In some embodiments, inhibiting ferroportin prevents iron from being exported and the iron is sequestered in the cells. In some embodiments, by inhibiting ferroportin, hepcidin prevents enterocytes from allowing iron into the hepatic portal system, thereby reducing dietary iron absorption. Hepcidin expression involves multiple aspects, including, for example, transcription of the HAMP gene, translation of the transcribed mRNA, and the posttranslational processing of the hepcidin precursor into the bioactive hepcidin-25 peptide (DTHFPICIFCCGCCHRSKCGMCCKT (SEQ ID NO: 129)). In some embodiments, hepcidin expression is modulated via the hemojuvelin-induced BMP signaling pathway. In some embodiments, hepcidin expression is modulated via the IL-6-JAK-STAT signaling pathway.

Hepcidin Antagonist: As used herein, a “hepcidin antagonist” refers to an agent that reduces hepcidin expression and/or hepcidin activity (directly or indirectly). In some embodiments, a hepcidin antagonist inhibits hepcidin-induced ferroportin degradation. Accordingly, in some embodiments, a hepcidin antagonist targets hepcidin function indirectly through the hepcidin stimulatory pathway to decrease hepcidin expression. In some embodiments, a hepcidin antagonist targets hepcidin function directly, e.g., by binding the hepcidin peptide to sequester free hepcidin or by binding ferroportin to inhibit the hepcidin-ferroportin binding interaction, thereby decreasing hepcidin-induced ferroportin degradation. In some embodiments, a hepcidin antagonist is a ferroportin inhibitor that disrupts ferroportin-hepcidin interactions, such as, for example, as disclosed in Ross S L, et al., Identification of Antibody and Small Molecule Antagonists of Ferroportin-Hepcidin Interaction. Front Pharmacol. 2017 Nov. 21; 8:838; Fung E., et al., High-Throughput Screening of Small Molecules Identifies Hepcidin Antagonists. Molecular Pharmacology March 2013, 83 (3) 681-690; and Angeliki Katsarou and Kostas Pantopoulos, Hepcidin Therapeutics. Pharmaceuticals (Basel). 2018 December; 11 (4): 127, the relevant contents of each of which are incorporated herein by reference.

Hematological recovery: As used herein, the term “hematological recovery,” refers to a process of bringing the oxygen carrying capacity in the blood of a subject to a state that is comparable to (e.g., equal to or in the range of) baseline values of a normal, healthy subject (e.g., a normal, healthy control subject). A “typical” range for baseline oxygen levels in the blood of a normal, healthy subject is between 95% and 100% oxygen saturation. In some embodiments, hematological recovery may be assessed by evaluating levels of one or more of erythropoiesis, circulating hemoglobin levels, reticulocyte hemoglobin (CHr), and mean corpuscular hemoglobin (MCH), levels of which are indicative of whether a subject has achieved a homeostatic oxygen carrying capacity.

Hemoglobin: As used herein, the term “hemoglobin (Hb)” refers to is the iron-containing oxygen-transport metalloprotein in red blood cells (erythrocytes). Hemoglobin in blood carries oxygen from the respiratory organs (e.g. lungs or gills) to the rest of the body (i.e. tissues). There it releases the oxygen to permit aerobic respiration to provide energy to power functions of an organism in the process called metabolism. A healthy individual human has about 12 to 20 g/dL of hemoglobin in blood. Hemoglobin (Hb) is synthesized in a complex series of steps. The heme part is synthesized in a series of steps in the mitochondria and the cytosol of immature red blood cells, while the globin protein parts are synthesized by ribosomes in the cytosol. Iron is an essential element for hemoglobin synthesis, particularly heme synthesis. The final step of heme synthesis is the addition of a iron ion to protoporphyrin IX, a precursor of heme, by ferrochelatase, thereby producing a heme molecule. Globin chain production occurs in the cytosol of erythrocytes and occurs by genetic transcription and translation. Many studies have shown that the presence of heme induces globin production. Heme combines with globin to form hemoglobin.

Hemojuvelin antagonist: As used herein, the term “hemojuvelin antagonist,” refers to a molecule that reduces expression of hemojuvelin or inhibits hemojuvelin activity, e.g., by binding to hemojuvelin. In some embodiments, the hemojuvelin antagonist is an antisense oligonucleotide (see, e.g., U.S. Pat. No. 7,534,764; U.S. Patent Publication No. US 2014/127325; and International Publication No. WO 2016/180784, which are incorporated herein by reference). In some embodiments, the hemojuvelin antagonist is an antibody (e.g., an anti-HJV antibody described herein, or an anti-HJV antibody described in WO2020086736, which is incorporated herein by reference). In other embodiments, the hemojuvelin antagonist is a small molecule compound that inhibits hemojuvelin, e.g., by competitive binding and/or chemical modification of hemojuvelin. In some embodiments, the hemojuvelin antagonist is a HJV fragment (see., e.g., an HJV fragment described by U.S. Pat. No. 8,507,435, which is incorporated herein by reference), or an HJV fusion protein (e.g., an HJV-Fc fusion protein described by U.S. Pat. No. 8,637,023, which is incorporated herein by reference).

HJV-induced BMP signaling: As used herein, the term “HJV-induced BMP signaling” refers to signaling through BMP receptors that is induced by Hemojuvelin (HJV), which is a membrane bound co-receptor for bone morphogenetic protein (BMP) signaling. As discussed in Xia Y, et al., Hemojuvelin regulates hepcidin expression via a selective subset of BMP ligands and receptors independently of neogenin, Blood. 2008 May 15; 111 (10): 5195-5204, in hepatocytes, HJV-induced BMP signaling positively regulates hepcidin mRNA expression. In some embodiments, HJV binds to BMP2, BMP4, BMP5, or BMP6 to induce BMP signaling, e.g., to positively regulate hepcidin levels in hepatocytes. In some embodiments, cleavage of HJV by matriptase-2 reduces the amount of cell surface HJV available to participate in BMP signaling. In some embodiments, induction of BMP signaling by HJV is independent of neogenin. However, in some embodiments, neogenin facilitates induction of BMP signaling by HJV, as discussed in Zhao et al, Neogenin Facilitates the Induction of Hepcidin Expression by Hemojuvelin in the Liver, J Biol Chem. 2016 Jun. 3; 291 (23): 12322-12335. In some embodiments, BMP6 is responsible for iron-dependent activation of the Smad signaling. In some embodiments, BMP6 is secreted from liver sinusoidal endothelial cells and binds to a BMP receptor (BMPR) on hepatocytes and thereby activates the SMAD signaling cascade. In such embodiments, HJV serves as a co-receptor for such BMP6, e.g., to positively regulate hepcidin levels in hepatocytes. In some embodiments, BMPs transduce signals by binding to one or a combination of type I and II serine/threonine kinase receptors. BMP type II receptors include BMPRII, ActRIIA, and ActRIIB. BMP type I receptors include ALK3, ALK6, and ALK2. In some embodiments, upon ligand binding, constitutively active type II receptors phosphorylate type I receptors, and type I receptors then phosphorylate intracellular receptor-activated Smads (R-Smads), namely Smad 1, Smad 5 and/or Smad 8. In such embodiments, activated R-Smads complex with the common partner Smad4 and translocate to the nucleus to regulate gene transcription, e.g., induction of hepcidin expression.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences (e.g., CDRs grafted in a heterologous framework).

Humanized antibody: The term “humanized antibody” refers to antibodies which comprise heavy and light chain variable region sequences from a non-human species (e.g., a mouse) but in which at least a portion of the V_{H} and/or V_{L} sequence has been altered to be more “human-like”, i.e., more similar to human germline variable sequences. One type of humanized antibody is a CDR-grafted antibody, in which human CDR sequences are introduced into non-human V_{H} and V_{L} sequences to replace the corresponding nonhuman CDR sequences. In one embodiment, humanized anti-hemojuvelin antibodies and antigen binding portions are provided. Such antibodies may be generated by obtaining murine anti-hemojuvelin monoclonal antibodies using traditional hybridoma technology followed by humanization using in vitro genetic engineering, such as those disclosed in Kasaian et al PCT publication No. WO 2005/123126 A2.

Iatrogenic blood loss: The term “iatrogenic blood loss”, as used herein, refers to condition in which a person has a blood loss condition due to medical interventions (including but not limited to phlebotomy, bleeding from medical procedures such as surgery, dilution of the blood by intravenous fluids). In some embodiments, the iatrogenic blood loss comprises phlebotomy. In some embodiments, a subject is undergoing or expected to undergo repeated phlebotomy due to repeated blood parameter monitoring. In some embodiments, a subject undergoing or expected to undergo repeated phlebotomy are susceptible to have a blood loss condition. In some embodiments, a subject is undergoing or expected to undergo a blood donation procedure (e.g., blood donation by phlebotomy). In some embodiments, the iatrogenic blood loss comprises a surgical procedure.

Isolated antibody: An “isolated antibody”, as used herein, is intended to refer to an antibody that is substantially free of other antibodies having different antigenic specificities (e.g., an isolated antibody that specifically binds hemojuvelin is substantially free of antibodies that specifically bind antigens other than hemojuvelin). An isolated antibody that specifically binds hemojuvelin may, however, have cross-reactivity to other antigens, such as other repulsive guidance molecule (RGM) proteins (e.g., RGMa and/or RGMb). Moreover, an isolated antibody may be substantially free of other cellular material and/or chemicals.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Mean corpuscular hemoglobin (MCH): As used herein, the term “mean corpuscular hemoglobin” refers to the average mass of hemoglobin (Hb) per red blood cell (RBC) in a sample of blood. It is calculated by dividing the total mass of hemoglobin by the number of red blood cells in a volume of blood.

MCH=(Hb*10)/RBC

In some embodiments, a normal MCH value in humans is 27 to 31 picograms (pg)/cell. The amount of hemoglobin per RBC depends on hemoglobin synthesis and the size of the RBC. The mass of the red cell is determined by the iron (as part of the hemoglobin molecule), thus MCH in picograms is roughly the mass of one red cell. In some embodiments, in iron deficiency anemia the cell mass becomes lighter, and a MCH below 27 pg is an indication of iron deficiency.

Recombinant antibody: The term “recombinant human antibody”, as used herein, is intended to include all human antibodies that are prepared, expressed, created or isolated by recombinant means, such as antibodies expressed using a recombinant expression vector transfected into a host cell (described in more details in this disclosure), antibodies isolated from a recombinant, combinatorial human antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for human immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. One embodiment of the disclosure provides fully human antibodies capable of binding human hemojuvelin which can be generated using techniques well known in the art, such as, but not limited to, using human Ig phage libraries such as those disclosed in Jermutus et al., PCT publication No. WO 2005/007699 A2.

Reticulocyte hemoglobin content (CHr): As used herein, the term “Reticulocyte hemoglobin content (CHr)”, refers to the hemoglobin level in reticulocytes, which are immature erythrocytes in circulation. The reticulocytes' hemoglobin content reflects the amount of iron available for hemoglobin production in the bone marrow. The reticulocyte hemoglobin content provides an indirect measure of the functional iron available for new red blood cell production over the previous several days (e.g., 3-4 days). In some embodiments, reticulocyte hemoglobin equivalent (Ret-He) is used in place of CHr (Toki et al., “Usefulness of Reticulocyte Hemoglobin Equivalent for Diagnosis of Iron Deficiency.” Blood. 2016; 128 (22): 3621).

Selective: As used herein, the term “selective” or “selectively” refers to the ability of a molecule to produce an effect in relation to its target molecule compared to a reference molecule. For example, a molecule that selectively inhibits its target molecule means that this molecule is capable of inhibiting its target molecule with a degree that is distinguishable from a reference molecule in an inhibition assay or other inhibitory context. For example, with respect to an inhibitor, the term, “selectively inhibits”, refers to the ability of the inhibitor to inhibit its target molecule with a degree that is distinguishable from a reference molecule that is not substantially inhibited in an inhibition assay, e.g., to an extent that permit selective inhibition of the target molecule, as described herein. For example, the half maximal inhibitory concentration (IC50) for the target molecule and/or the reference molecule can be tested in a kinase potency assay as described in Asshoff, M. et al., Momelotinib inhibits ACVR1/ALK2, decreases hepcidin production, and ameliorates anemia of chronic disease in rodents. Blood. 2017 Mar. 30; 129 (13): 1823-1830 (e.g., Kinase potency assay by Carna Biosciences). In this assay, inhibitor solution (e.g., solution containing the selective inhibitor to be tested)/kinase substrate is mixed with target molecule solution (e.g., ALK2) or reference molecule solution (e.g., JAK1 or JAK2), and incubated under room temperature for 1 hour. Once the reaction is terminated, the signal produced by enzymatic activity on the substrate can be measured. The half maximal inhibitor concentration for the target molecule and the reference molecule can be calculated. In some embodiments, a molecule described herein selectively binds to a target molecule. In some embodiments, a molecule described herein selectively inhibits to a target molecule. In some embodiments, a molecule described herein selectively antagonizes to a target molecule. In some embodiments, a molecule described herein selectively neutralizes to a target molecule.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to hemojuvelin.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a blood loss condition.

Transferrin Saturation (TSAT %): As used herein, the term “transferrin saturation (TSAT %)”, refers to a percentage value of serum iron divided by the total iron-binding capacity of the available transferrin, the main protein that binds iron in the blood. This value indicates how much serum iron is bound to transferrin. For instance, a value of 15% means that 15% of iron-binding sites of transferrin are being occupied by iron. A low transferrin saturation is a common indicator of iron deficiency whereas a high transferrin saturation may indicate iron overload or hemochromatosis. Transferrin saturation is also called transferrin saturation index (TSI) or transferrin saturation percentage (TS %).

Treatment: As used herein, the term “treating” or “treatment” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder. Alleviating a target disease/disorder includes delaying or preventing the development or progression of the disease, or reducing disease severity.

### II. Methods for Treating Blood Loss Conditions

The present disclosure, at least in part, provides compositions and methods for treating a subject having a blood loss condition, the method comprising administering the subject a hemojuvelin antagonist. In some embodiments, the hemojuvelin antagonist is administered in an amount effective for promoting hematological recovery. In some embodiments, the blood loss condition causes the subject to lose red blood cells (RBCs). In some embodiments, when blood is lost, the body pulls water from tissues outside the bloodstream in an attempt to keep the blood vessels filled. As a result, the blood is diluted, and the hematocrit (the percentage of red blood cells in the total amount of blood in the body, or blood volume) is reduced. Eventually, increased production of red blood cells by the bone marrow may correct the blood loss condition. However, over time, bleeding reduces the amount of iron in the body, so that the bone marrow is not able to increase production of new red blood cells to replace those lost. In some embodiments, loss of red blood cell and iron by bleeding reduces the oxygen carrying capacity of the subject. In such cases, additional therapy may be needed to promote hematological recovery of these subjects. Accordingly, aspects of the present disclosure relate to methods for treating a subject having a blood loss condition by administering an hemojuvelin antagonist, in an amount effective for promoting hematological recovery. In some embodiments, the hematological recovery comprises recovery of erythropoiesis, reticulocyte hemoglobin content (CHr), mean corpuscular hemoglobin (MCH) and/or circulating hemoglobin levels to baseline levels.

In some embodiments, the hematological recover is achieved in a subject receiving the hemojuvelin antagonist (e.g., an anti-HJV antibody) treatment within a shorter duration than would be achieved by a control subject who did not receive the hemojuvelin antagonist (e.g., an anti-HJV antibody). In some embodiments, the hematological recovery is achieved in a subject receiving the hemojuvelin antagonist (e.g., an anti-HJV antibody) treatment within 1 day, 2 days, 3 days, 4 days, 5 days, 6 days, one week, two weeks, three weeks, one month, two months, three months, four months, five months, six months, seven months, eight months, nine months, ten months, eleven months, 1 year, 2 years, or 3 years. In some embodiments, the hematological recovery is achieved in a subject receiving the hemojuvelin antagonist (e.g., an anti-HJV antibody) treatment 3 days, 4 days, 5 days, 6 days, one week, two weeks, three weeks, one month, two months, three months, four months, five months, six months, seven months, eight months, nine months, ten months, eleven months, or 1 year shorter than would be achieved by a control subject who did not receive the hemojuvelin antagonist (e.g., an anti-HJV antibody).

In some embodiments, administering a subject having a blood loss condition an hemojuvelin antagonist (e.g., an anti-HJV antibody) promotes recovery of erythropoiesis. In some embodiments, recovery of erythropoiesis results in increase of RBC count to baseline level. In some embodiments, recovery of erythropoiesis results in increase of RBC count to baseline level within 1 day, 2 days, 3 days, 4 days, 5 days, 6 days, one week, two weeks, three weeks, one month, two months, three months, four months, five months, six months, seven months, eight months, nine months, ten months, eleven months, 1 year, 2 years, or 3 years.

In some embodiments, administering to a subject having a blood loss condition an hemojuvelin antagonist (e.g., an anti-HJV antibody) promotes recovery of reticulocyte hemoglobin content (CHr). In some embodiments, recovery of CHr results in increase of CHr to baseline level. In some embodiments, recovery of CHr results in increase of CHr to baseline level within 1 day, 2 days, 3 days, 4 days, 5 days, 6 days, one week, two weeks, three weeks, one month, two months, three months, four months, five months, six months, seven months, eight months, nine months, ten months, eleven months, 1 year, 2 years, or 3 years.

In some embodiments, administering a subject having a blood loss condition an hemojuvelin antagonist (e.g., an anti-HJV antibody) promotes recovery of mean corpuscular hemoglobin (MCH). In some embodiments, recovery of MCH results in increase of MCH to baseline level. In some embodiments, recovery of MCH results in increase of MCH to baseline level within 1 day, 2 days, 3 days, 4 days, 5 days, 6 days, one week, two weeks, three weeks, one month, two months, three months, four months, five months, six months, seven months, eight months, nine months, ten months, eleven months, 1 year, 2 years, or 3 years.

In some embodiments, administering a subject having a blood loss condition an hemojuvelin antagonist (e.g., an anti-HJV antibody) promotes recovery of circulating hemoglobin level. In some embodiments, recovery of circulating hemoglobin level results in increase of circulating hemoglobin level to baseline level. In some embodiments, recovery of circulating hemoglobin level results in increase of circulating hemoglobin level to baseline level within 1 day, 2 days, 3 days, 4 days, 5 days, 6 days, one week, two weeks, three weeks, one month, two months, three months, four months, five months, six months, seven months, eight months, nine months, ten months, eleven months, 1 year, 2 years, or 3 years.

In some embodiments, the blood loss condition comprises acute blood loss. In some embodiments, acute loss of blood due to an injury, surgery, childbirth, or a ruptured blood vessel may lead to loss of large amounts of blood suddenly. In some embodiments, acute loss of between 30% and 40% of total blood volume requires blood transfusion, and acute loss of more than 40% of total blood volume is life threatening and requires immediate blood transfusion. In some embodiments, the subject has an acute blood loss condition in which the subject lost more than 30% of total blood volume (e.g., 30%, 31%, 32%, 33%, 34%, 35%, 36%, 37%, 38%, 39%, 40%, or more) and is identified as requiring blood transfusion. In some embodiments, subjects having an acute blood loss condition resulting in blood loss of less than 30% (e.g., less than 30%, less than 25%, less than 20%, less than 15%, less than 10%, less than 9%, less than 8%, less than 7%, less than 6%, less than 5%, less than 4%, less than 3%, less than 2%, less than 1%) of total blood volume does not require blood transfusion, and can be treated with the hemojuvelin antagonist (e.g., anti-HJV antibody). In some embodiments, the subject having an acute blood loss condition in which the subject lost blood in the range of 1%-30% (e.g., 1%-30%, 1%-25%, 1%-20%, 1%-15%, 1%-10%, 1%-9%, 1%-8%, 1%-7%, 1%-6%, 1%-5%, 1%-4%, 1%-3%, 1%-2%, 2%-30%, 2%-25%, 2%-20%, 2%-15%, 2%-10%, 2%-9%, 2%-8%, 2%-7%, 2%-6%, 2%-5%, 2%-4%, 2%-3%, 3%-30%, 3%-25%, 3%-20%, 3%-15%, 3%-10%, 3%-9%, 3%-8%, 3%-7%, 3%-6%, 3%-5%, 3%-4%, 4%-30%, 4%-25%, 4%-20%, 4%-15%, 4%-10%, 4%-9%, 4%-8%, 4%-7%, 4%-6%, 4%-5%, 5%-30%, 5%-25%, 5%-20%, 5%-15%, 5%-10%, 5%-9%, 5%-8%, 5%-7%, 5%-6%, 6%-30%, 6%-25%, 6%-20%, 6%-15%, 6%-10%, 6%-9%, 6%-8%, 6%-7%, 7%-30%, 7%-25%, 7%-20%, 7%-15%, 7%-10%, 7%-9%, 7%-8%, 8%-30%, 8%-25%, 8%-20%, 8%-15%, 8%-10%, 8%-9%, 9%-30%, 9%-25%, 9%-20%, 9%-15%, 9%-10%, 10%-30%, 10%-25%, 10%-20%, 10%-15%, 15%-30%, 15%-25%, 15%-20%, 20%-30%, 20%-25%, 25%-30% of total blood volume does not require blood transfusion, and can be treated with the hemojuvelin antagonist (e.g., anti-HJV antibody). In some embodiments, the subject having an acute blood loss condition in which the subject lost blood in the range of 1%-10% of total blood volume does not require blood transfusion, and can be treated with the hemojuvelin antagonist (e.g., anti-HJV antibody).

In some embodiments, the subjects has an acute blood loss condition and has a hemoglobin level of less than 7 g/dL (e.g., less than 7 g/dL, less than 6.5 g/dL, less than 6 g/dL, less than 5.5 g/dL, less than 5 g/dL, less than 4.5 g/dL, less than 5.5 g/dL, less than 5 g/dL, less than 4.5 g/dL, less than 4 g/dL, less than 3.5 g/dL, less than 3 g/dL, less than 2.5 g/dL, or less than 2 g/dL), and the subject is identified as requiring blood transfusion. In some embodiments, the blood transfusion increases the subject's hemoglobin level to at least 7 g/dL. In some embodiments, the blood transfusion increases the subject's hemoglobin level to the range of 7-10 g/dL (e.g., 7-8 g/dL, 7-9 g/dL, 7.5-8.5 g/dL, 7.5-9.5 g/dL, 7-7.5 g/dL, 7-8.5 g/dL, 7-9.5 g/dL, 7.5-8 g/dL, 7.5-9 g/dL, 7.5-10 g/dL, 8-8.5 g/dL, 8-9 g/dL, 8-9.5 g/dL, 8-10 g/dL, 9-9.5 g/dL, or 9-10 g/dL), and the subject can be treated with an hemojuvelin antagonist (e.g., an anti-HJV antibody) to promote further hematological recovery. In some embodiments, the subject has an acute blood loss condition and has a hemoglobin level of more than 7 g/dL, and the subject is identified as not requiring blood transfusion. In some embodiments, the subjects has an acute blood loss condition and has a hemoglobin level to the range of 7-10 g/dL (e.g., 7-8 g/dL, 7-9 g/dL, 7.5-8.5 g/dL, 7.5-9.5 g/dL, 7-7.5 g/dL, 7-8.5 g/dL, 7-9.5 g/dL, 7.5-8 g/dL, 7.5-9 g/dL, 7.5-10 g/dL, 8-8.5 g/dL, 8-9 g/dL, 8-9.5 g/dL, 8-10 g/dL, 9-9.5 g/dL, or 9-10 g/dL), and the subject is identified as not requiring blood transfusion and can be treated with an hemojuvelin antagonist (e.g., an anti-HJV antibody) to promote hematological recovery.

In some embodiments, the blood loss condition comprises chronic blood loss (e.g., the blood loss occurs over a long period of time). Chronic blood loss may occur from various parts of the body. In some embodiments, chronic blood loss is obvious and noticeable (e.g., nose bleeding, hemorrhoids bleeding, upper gastrointestinal tract bleeding). In some embodiments, chronic blood loss is not noticeable due to the small amounts of bleeding but over time, a significant amount blood is lost. In some embodiments, a subject having chronic loss of more than 30% of total blood volume (e.g., between 30% and 35%, between 30% and 40%, between 30% and 45%, between 30% and 50%, between 30% and 55%, between 30% and 60%, between 40% and 45%, between 40% and 50%, between 40% and 55%, between 40% and 60%, between 50% and 55%, between 50% and 60%, or between 55% and 40%) may only experience mild symptom (e.g., fatigue, weakness, black stool, blood in urine) and the subject may not require blood transfusion, and can be treated with an hemojuvelin antagonist (e.g., anti-HJV antibody). In some embodiments, a subject having a chronic blood loss condition resulting in blood loss of less than 30% (e.g., less than 30%, less than 25%, less than 20%, less than 15%, less than 10%, less than 9%, less than 8%, less than 7%, less than 6%, less than 5%, less than 4%, less than 3%, less than 2%, less than 1%) of total blood volume does not require blood transfusion, and can be treated with the hemojuvelin antagonist (e.g., anti-HJV antibody). In some embodiments, the subject having a chronic blood loss condition in which the subject lost blood in the range of 1%-30% (e.g., 1%-30%, 1%-25%, 1%-20%, 1%-15%, 1%-10%, 1%-9%, 1%-8%, 1%-7%, 1%-6%, 1%-5%, 1%-4%, 1%-3%, 1%-2%, 2%-30%, 2%-25%, 2%-20%, 2%-15%, 2%-10%, 2%-9%, 2%-8%, 2%-7%, 2%-6%, 2%-5%, 2%-4%, 2%-3%, 3%-30%, 3%-25%, 3%-20%, 3%-15%, 3%-10%, 3%-9%, 3%-8%, 3%-7%, 3%-6%, 3%-5%, 3%-4%, 4%-30%, 4%-25%, 4%-20%, 4%-15%, 4%-10%, 4%-9%, 4%-8%, 4%-7%, 4%-6%, 4%-5%, 5%-30%, 5%-25%, 5%-20%, 5%-15%, 5%-10%, 5%-9%, 5%-8%, 5%-7%, 5%-6%, 6%-30%, 6%-25%, 6%-20%, 6%-15%, 6%-10%, 6%-9%, 6%-8%, 6%-7%, 7%-30%, 7%-25%, 7%-20%, 7%-15%, 7%-10%, 7%-9%, 7%-8%, 8%-30%, 8%-25%, 8%-20%, 8%-15%, 8%-10%, 8%-9%, 9%-30%, 9%-25%, 9%-20%, 9%-15%, 9%-10%, 10%-30%, 10%-25%, 10%-20%, 10%-15%, 15%-30%, 15%-25%, 15%-20%, 20%-30%, 20%-25%, 25%-30% of total blood volume does not require blood transfusion, and can be treated with the hemojuvelin antagonist (e.g., anti-HJV antibody). In some embodiments, the subject having a chronic blood loss condition in which the subject lost blood in the range of 1%-10% of total blood volume does not require blood transfusion, and can be treated with the hemojuvelin antagonist (e.g., anti-HJV antibody).

Non-limiting examples of diseases that may lead to chronic blood loss include gastrointestinal (GI) disease (e.g., esophageal varices, gastritis, gastric ulcer, duodenal ulcer, diverticulosis, Meckel's diverticulum, intestinal polyps, inflammatory bowel disease (IBD), celiac disease or colorectal cancer), a genitourinary disease (e.g., menorrhagia, fibroid, endometriosis, bladder tumors, or renal stones), an infectious disease (e.g., Dengue fever, Ebola virus disease, Lassa fever, Marburg virus disease, yellow fever, sepsis, bacterial vaginosis, Lemierre's syndrome, and tuberculosis, malaria, Trichuriasis, or Schistosomiasis). In some embodiments, the subject having a chronic blood loss condition can be treated with an hemojuvelin antagonist (e.g., an anti-HJV antibody) to promote hematological recovery.

In some embodiments, the blood loss condition comprises iatrogenic blood loss. In some embodiments, the blood loss condition (e.g., iatrogenic blood loss) comprises a phlebotomy procedure. As used herein, a phlebotomy procedure comprises any procedure in which a needle is used to take blood from a vein. In some embodiments, the blood loss condition (e.g., iatrogenic blood loss) comprises a blood transfusion procedure. In some embodiments, the blood loss condition (e.g., iatrogenic blood loss) comprises a blood donation procedure. As used herein, a blood donation procedure comprises any procedure wherein a subject voluntarily has blood drawn to be used for a medical procedure, such as a blood transfusion. In some embodiments, the blood loss condition (e.g., iatrogenic blood loss) comprises a surgical procedure. As used herein, a surgical procedure comprises any medical procedure involving an incision with instruments.

In some embodiments, the blood loss condition comprises a bleeding wound. A wound is a type of damage or breakage to a tissue (e.g., external tissue or internal tissue).

In some embodiments, the bleeding wound be an internal bleeding wound. In some embodiments, the internal bleed wound comprises ruptured blood vessels, organ contusion, or hematoma. In some embodiments, the internal bleeding comprises ruptured blood vessels including but not limited to ruptured artery, ruptured vein, or ruptured capillary, for example, caused by varices, stroke, subarachnoid hemorrhage, aneurysm, or ruptured aorta. In some embodiments, the internal bleeding comprises organ contusion. Organ contusion refers to bruises in internal organs (e.g., lungs, kidneys, heart, bones, spleen, intestines, brain, or muscle). In some embodiments, organ contusion causes bleeding in the damaged organ. In some embodiments, the internal bleeding comprises hematoma. In some embodiments, a hematoma may be a collection (or pooling) of blood outside the blood vessel. In some embodiments, hematomas include but are not limited to subdural hematoma, spinal hematoma, subungual hematoma, or hepatic hematoma.

In some embodiments, the bleeding wound is an external bleeding wound. An external wound is damage or breakage on the surface of the skin. An external bleeding wound includes but is not limited to a cut, a stab, a puncture, an avulsion, an incision, and a penetration, for example, by a knife, a gun, a needle, a lancet, an external force, or an automobile accident.

One of ordinary skill in the art would be able to determine whether a subject with an internal bleeding wound and/or an external bleeding wound requires blood transfusion prior to receiving the hemojuvelin antagonist (e.g., an anti-HJV antibody) based on the existing technique to assess the condition of the subject (e.g., amount of blood lost and/or the hemoglobin level of the subject).

In some embodiments, the blood loss condition comprises a disease. In some embodiments, the disease is a gastrointestinal (GI) disease. As used herein, a GI disease is any disease/disorder that affects normal GI function. Examples of GI diseases include, but are not limited to, esophageal varices, gastritis, gastric ulcers, duodenal ulcers, diverticulosis, Meckel's diverticulum, intestinal polyps, inflammatory bowel disease (IBD), hemorrhoids, celiac disease, and colorectal cancer. Gastrointestinal (GI) diseases may present with GI bleeding, usually due to a disorder in the digestive tract of a subject. In some embodiments, the blood often appears in stool (e.g., fresh blood in stool or black tarry stool) or vomit (e.g., bright red vomit or dark brown vomit). In some embodiments, the blood is not visible. The level of bleeding can range from mild to severe and can be life-threatening. In some embodiments, the GI disease comprises blood loss due to upper GI bleeding. In some embodiments, the upper GI bleeding comprises esophageal varices, gastric ulcers, duodenal ulcer, esophagitis, or Mallory-Weiss tears. In some embodiments, the GI disease comprises blood loss due to lower GI bleeding. In some embodiments, the lower GI bleeding comprises diverticulosis, diverticulitis, Meckel's diverticulum, intestinal polyps, inflammatory bowel disease (IBD), celiac disease, colorectal cancer, anal fissures, or proctitis. In some embodiments, the subject having a blood loss condition comprising a GI disease is identified as requiring blood transfusion, and followed by an hemojuvelin antagonist (e.g., an anti-HJV antibody) treatment (e.g., to promote hematological recovery). In some embodiments, the subject having a blood loss condition comprising a GI disease is not identified as requiring blood transfusion, and is treated with an hemojuvelin antagonist (e.g., an anti-HJV antibody) (e.g., to promote hematological recovery). Determining whether a subject requires a blood transfusion may be done by any method known in the art and commonly practiced in medical clinics (see, e.g., Zomorrodi et al., “Determining the Need for Blood Transfusion.” In: StatPearls. Treasure Island (FL): StatPearls Publishing; Oct. 17, 2022).

In some embodiments, the blood loss condition comprises a genitourinary disease, which are diseases of the reproductive system and the urinary system. In some embodiments, the disease is a urinary tract disease, and the blood appears in the urine. In some embodiments, the urinary tract disease comprises bladder tumors, urinary tract infection (UTI), urinary tract laceration, or renal stones. In some embodiments, the disease is a reproductive organ disease. In some embodiments, the reproductive organ disease comprises menorrhagia, fibroid, bleeding during pregnancy, ectopic pregnancy, miscarriage, endometrial hyperplasia, or endometriosis.

In some embodiments, the blood loss condition comprises an infectious disease. As used herein, an infectious disease includes any disease, disorder, or illness caused by bacteria, fungi, parasites, or viruses. In some embodiments, the infectious disease comprises blood loss due to viral hemorrhagic fevers (VHFs), which are a group of infectious diseases caused by viruses. These infections can damage the blood vessels and cause dangerous bleeding in organs throughout the body. In some embodiments, viral hemorrhagic fevers (VHFs) comprise, but are not limited to, Dengue fever, Ebola virus disease, Lassa fever, Hantavirus pulmonary syndrome, Marburg virus disease, or yellow fever. In some embodiments, the infectious disease comprises blood loss due to a bacterial infectious disease. In some embodiments, the bacterial infectious diseases comprise sepsis, bacterial vaginosis, Lemierre's syndrome, or tuberculosis. In some embodiments, the infectious disease comprises blood loss due to a parasitic infectious disease. In some embodiments, the parasitic infectious disease comprises malaria, Trichuriasis, and Schistosomiasis.

In some embodiments, the blood loss condition comprises persistent blood loss. Persistent blood loss refers to blood loss that is constant without stopping over a long period of time (e.g., one month, two months, three months, four months, five months, six months, seven months, eight months, nine months, ten months, eleven months, twelve months, one year, two years, three years, four years, five years, or longer). In some embodiments, the persistent blood loss cause the subject to lose blood in the range of 1%-30% (e.g., 1%-30%, 1%-25%, 1%-20%, 1%-15%, 1%-10%, 1%-9%, 1%-8%, 1%-7%, 1%-6%, 1%-5%, 1%-4%, 1%-3%, 1%-2%, 2%-30%, 2%-25%, 2%-20%, 2%-15%, 2%-10%, 2%-9%, 2%-8%, 2%-7%, 2%-6%, 2%-5%, 2%-4%, 2%-3%, 3%-30%, 3%-25%, 3%-20%, 3%-15%, 3%-10%, 3%-9%, 3%-8%, 3%-7%, 3%-6%, 3%-5%, 3%-4%, 4%-30%, 4%-25%, 4%-20%, 4%-15%, 4%-10%, 4%-9%, 4%-8%, 4%-7%, 4%-6%, 4%-5%, 5%-30%, 5%-25%, 5%-20%, 5%-15%, 5%-10%, 5%-9%, 5%-8%, 5%-7%, 5%-6%, 6%-30%, 6%-25%, 6%-20%, 6%-15%, 6%-10%, 6%-9%, 6%-8%, 6%-7%, 7%-30%, 7%-25%, 7%-20%, 7%-15%, 7%-10%, 7%-9%, 7%-8%, 8%-30%, 8%-25%, 8%-20%, 8%-15%, 8%-10%, 8%-9%, 9%-30%, 9%-25%, 9%-20%, 9%-15%, 9%-10%, 10%-30%, 10%-25%, 10%-20%, 10%-15%, 15%-30%, 15%-25%, 15%-20%, 20%-30%, 20%-25%, 25%-30% of total blood volume, and the subject does not require blood transfusion, and can be treated with the hemojuvelin antagonist (e.g., anti-HJV antibody).

In some embodiments, the blood loss condition comprises intermittent blood loss. Intermittent blood loss refers to blood loss that occurs in an interval. In some embodiments, the intermittent blood loss occurs on a regular interval (e.g., up to 1 hour, up to 4 hours, up to 8 hours, up to 12 hours, up to 16 hours, up to 20 hours, up to one day apart, up to two days apart, up to three days apart, up to five days apart, up to one week apart, up to two weeks apart, up to one month apart). In some embodiments, the intermittent blood loss occurs on irregular intervals (e.g., one day apart during the first and second bleeding instances, but one week apart between the second and third bleeding instances). In some embodiments, the intermittent blood loss cause the subject to lose blood in the range of 1%-30% (e.g., 1%-30%, 1%-25%, 1%-20%, 1%-15%, 1%-10%, 1%-9%, 1%-8%, 1%-7%, 1%-6%, 1%-5%, 1%-4%, 1%-3%, 1%-2%, 2%-30%, 2%-25%, 2%-20%, 2%-15%, 2%-10%, 2%-9%, 2%-8%, 2%-7%, 2%-6%, 2%-5%, 2%-4%, 2%-3%, 3%-30%, 3%-25%, 3%-20%, 3%-15%, 3%-10%, 3%-9%, 3%-8%, 3%-7%, 3%-6%, 3%-5%, 3%-4%, 4%-30%, 4%-25%, 4%-20%, 4%-15%, 4%-10%, 4%-9%, 4%-8%, 4%-7%, 4%-6%, 4%-5%, 5%-30%, 5%-25%, 5%-20%, 5%-15%, 5%-10%, 5%-9%, 5%-8%, 5%-7%, 5%-6%, 6%-30%, 6%-25%, 6%-20%, 6%-15%, 6%-10%, 6%-9%, 6%-8%, 6%-7%, 7%-30%, 7%-25%, 7%-20%, 7%-15%, 7%-10%, 7%-9%, 7%-8%, 8%-30%, 8%-25%, 8%-20%, 8%-15%, 8%-10%, 8%-9%, 9%-30%, 9%-25%, 9%-20%, 9%-15%, 9%-10%, 10%-30%, 10%-25%, 10%-20%, 10%-15%, 15%-30%, 15%-25%, 15%-20%, 20%-30%, 20%-25%, 25%-30% of total blood volume in each bleeding instance, and the subject does not require blood transfusion, and can be treated with the hemojuvelin antagonist (e.g., anti-HJV antibody).

In some aspects, the present disclosure relates to composition and methods for treating a subject having a blood loss condition. In some embodiments, any of the blood loss condition suitable for a hemojuvelin antagonist (e.g., an anti-HJV antibody) treatment may be characterized with one or more of the hematological standards described herein. In some embodiments, the blood loss condition may be characterized as a mild to moderate blood loss condition or a severe blood loss condition in accordance with appropriate diagnostic threshold parameters.

In some embodiments, the blood loss condition is characterized based on a level of circulating hemoglobin (Hgb), wherein the severity of the blood loss condition increases with decreasing levels of circulating Hgb. In some embodiments, circulating hemoglobin levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment circulating hemoglobin levels in the subject. In some embodiments, prior to administration, a subject is not identified as requiring blood transfusion and the blood loss condition suitable for a hemojuvelin antagonist (e.g., anti-HJV antibody) is associated with circulating hemoglobin levels of at least 6 g/dL (e.g., at least 6 g/dL, at least 6.5 g/dL, at least 7 g/dL, at least 7.5 g/dL, at least 8 g/dL, at least 8.5 g/dL, at least 9 g/dL, at least 9.5 g/dL, at least 10 g/dL, at least 10.5 g/dL, at least 11 g/dL, at least 11.5 g/dL, at least 12 g/dL, at least 12.5 g/dL, or at least 13 g/dL). In some embodiments, prior to administration, a subject is not identified as requiring blood transfusion and the blood loss condition suitable for a hemojuvelin antagonist (e.g., anti-HJV antibody) is associated with circulating hemoglobin levels in the range of 6-13 g/dL, 7-13 g/dL, 7-12.5 g/dL, 7-12 g/dL, 7-11.5 g/dL, 7-11 g/dL, 7-10.5 g/dL, 7-10 g/dL, 7-9.5 g/dL, 7-9 g/dL, 7-8.5 g/dL, 7-8 g/dL, 7-7.5 g/dL, 7.5-13 g/dL, 7.5-12.5 g/dL, 7.5-12 g/dL, 7.5-11.5 g/dL, 7.5-11 g/dL, 7.5-10.5 g/dL, 7.5-10 g/dL, 7.5-9.5 g/dL, 7.5-9 g/dL, 7.5-8.5 g/dL, 7.5-8 g/dL, 8-13 g/dL, 8-12.5 g/dL, 8-12 g/dL, 8-11.5 g/dL, 8-11 g/dL, 8-10.5 g/dL, 8-10 g/dL, 8-9.5 g/dL, 8-9 g/dL, 8-8.5 g/dL, 8.5-13 g/dL, 8.5-12.5 g/dL, 8.5-12 g/dL, 8.5-11.5 g/dL, 8.5-11 g/dL, 8.5-10.5 g/dL, 8.5-10 g/dL, 8.5-9.5 g/dL, 8.5-9 g/dL, 9-13 g/dL, 9-12.5 g/dL, 9-12 g/dL, 9-11.5 g/dL, 9-11 g/dL, 9-10.5 g/dL, 9-10 g/dL, 9-9.5 g/dL, 10-13 g/dL, 10-12.5 g/dL, 10-12 g/dL, 10-11.5 g/dL, 10-11 g/dL, 10-10.5 g/dL, 10.5-13 g/dL, 10.5-12.5 g/dL, 10.5-12 g/dL, 10.5-11.5 g/dL, 10.5-11 g/dL, 11-13 g/dL, 11-12.5 g/dL, 11-12 g/dL, 11-11.5 g/dL, 11.5-13 g/dL, 11.5-12.5 g/dL, 11.5-12 g/dL, 12-13 g/dL, 12-12.5 g/dL, 12.5-13 g/dL, 6.5-13 g/dL, 6.5-12.5 g/dL, 6.5-12 g/dL, 6.5-11.5 g/dL, 6.5-11 g/dL, 6.5-10.5 g/dL, 6.5-10 g/dL, 6.5-9.5 g/dL, 6.5-9 g/dL, 6.5-8.5 g/dL, 6.5-8 g/dL, 6.5-7.5 g/dL, 6.5-7 g/dL, 6-13 g/dL, 6-12.5 g/dL, 6-12 g/dL, 6-11.5 g/dL, 6-11 g/dL, 6-10.5 g/dL, 6-10 g/dL, 6-9.5 g/dL, 6-9 g/dL, 6-8.5 g/dL, 6-8 g/dL, 6-7.5 g/dL, 6-7 g/dL, or 6-6.5 g/dL. In some embodiments, prior to administration, a subject is identified as requiring blood transfusion according to the circulating hemoglobin levels described elsewhere herein such that the blood transfusion increases the hemoglobin level in the subject to at least 6 g/dL (e.g., at least 6 g/dL, at least 6.5 g/dL, at least 7 g/dL, at least 7.5 g/dL, at least 8 g/dL, at least 8.5 g/dL, at least 9 g/dL, at least 9.5 g/dL, at least 10 g/dL, at least 10.5 g/dL, at least 11 g/dL, at least 11.5 g/dL, at least 12 g/dL, at least 12.5 g/dL, at least 13 g/dL, in the range of 6-13 g/dL, 7-13 g/dL, 7-12.5 g/dL, 7-12 g/dL, 7-11.5 g/dL, 7-11 g/dL, 7-10.5 g/dL, 7-10 g/dL, 7-9.5 g/dL, 7-9 g/dL, 7-8.5 g/dL, 7-8 g/dL, 7-7.5 g/dL, 7.5-13 g/dL, 7.5-12.5 g/dL, 7.5-12 g/dL, 7.5-11.5 g/dL, 7.5-11 g/dL, 7.5-10.5 g/dL, 7.5-10 g/dL, 7.5-9.5 g/dL, 7.5-9 g/dL, 7.5-8.5 g/dL, 7.5-8 g/dL, 8-13 g/dL, 8-12.5 g/dL, 8-12 g/dL, 8-11.5 g/dL, 8-11 g/dL, 8-10.5 g/dL, 8-10 g/dL, 8-9.5 g/dL, 8-9 g/dL, 8-8.5 g/dL, 8.5-13 g/dL, 8.5-12.5 g/dL, 8.5-12 g/dL, 8.5-11.5 g/dL, 8.5-11 g/dL, 8.5-10.5 g/dL, 8.5-10 g/dL, 8.5-9.5 g/dL, 8.5-9 g/dL, 9-13 g/dL, 9-12.5 g/dL, 9-12 g/dL, 9-11.5 g/dL, 9-11 g/dL, 9-10.5 g/dL, 9-10 g/dL, 9-9.5 g/dL, 10-13 g/dL, 10-12.5 g/dL, 10-12 g/dL, 10-11.5 g/dL, 10-11 g/dL, 10-10.5 g/dL, 10.5-13 g/dL, 10.5-12.5 g/dL, 10.5-12 g/dL, 10.5-11.5 g/dL, 10.5-11 g/dL, 11-13 g/dL, 11-12.5 g/dL, 11-12 g/dL, 11-11.5 g/dL, 11.5-13 g/dL, 11.5-12.5 g/dL, 11.5-12 g/dL, 12-13 g/dL, 12-12.5 g/dL, 12.5-13 g/dL, 6.5-13 g/dL, 6.5-12.5 g/dL, 6.5-12 g/dL, 6.5-11.5 g/dL, 6.5-11 g/dL, 6.5-10.5 g/dL, 6.5-10 g/dL, 6.5-9.5 g/dL, 6.5-9 g/dL, 6.5-8.5 g/dL, 6.5-8 g/dL, 6.5-7.5 g/dL, 6.5-7 g/dL, 6-13 g/dL, 6-12.5 g/dL, 6-12 g/dL, 6-11.5 g/dL, 6-11 g/dL, 6-10.5 g/dL, 6-10 g/dL, 6-9.5 g/dL, 6-9 g/dL, 6-8.5 g/dL, 6-8 g/dL, 6-7.5 g/dL, 6-7 g/dL, or 6-6.5 g/dL), and the subject can be subsequently treated with an hemojuvelin antagonist (e.g., anti-HJV antibody) to promote hematological recovery.

In some embodiments, after administration of an hemojuvelin antagonist (e.g., an anti-HJV antibody), the subject's circulating hemoglobin level increases. In some embodiments, after administration of an hemojuvelin antagonist (e.g., an anti-HJV antibody), the subject's circulating hemoglobin level increases to baseline level before the blood loss condition. In some embodiments, after administration of an hemojuvelin antagonist (e.g., an anti-HJV antibody), the subject's circulating hemoglobin level increases by 1-5 g/dL, 1-4.5 g/dL, 1-4 g/dL, 1-3.5 g/dL, 1-3 g/dL, 1-2.5 g/dL, 1-2 g/dL, 1-1.5 g/dL, 1.5-5 g/dL, 1.5-4.5 g/dL, 1.5-4 g/dL, 1.5-3.5 g/dL, 1.5-3 g/dL, 1.5-2.5 g/dL, 1.5-2 g/dL, 2-5 g/dL, 2-4.5 g/dL, 2-4 g/dL, 2-3.5 g/dL, 2-3 g/dL, 2-2.5 g/dL, 2.5-5 g/dL, 2.5-4.5 g/dL, 2.5-4 g/dL, 2.5-3.5 g/dL, 2.5-3 g/dL, 3-5 g/dL, 3-4.5 g/dL, 3-4 g/dL, 3-3.5 g/dL, 3.5-5 g/dL, 3.5-4.5 g/dL, 3.5-4 g/dL, 4-5 g/dL, 4-4.5 g/dL, or 4.5-5 g/dL. In some embodiments, after administration of an hemojuvelin antagonist (e.g., an anti-HJV antibody), the subject's circulating hemoglobin level increases by 1-5 g/dL, 1-4.5 g/dL, 1-4 g/dL, 1-3.5 g/dL, 1-3 g/dL, 1-2.5 g/dL, 1-2 g/dL, 1-1.5 g/dL, 1.5-5 g/dL, 1.5-4.5 g/dL, 1.5-4 g/dL, 1.5-3.5 g/dL, 1.5-3 g/dL, 1.5-2.5 g/dL, 1.5-2 g/dL, 2-5 g/dL, 2-4.5 g/dL, 2-4 g/dL, 2-3.5 g/dL, 2-3 g/dL, 2-2.5 g/dL, 2.5-5 g/dL, 2.5-4.5 g/dL, 2.5-4 g/dL, 2.5-3.5 g/dL, 2.5-3 g/dL, 3-5 g/dL, 3-4.5 g/dL, 3-4 g/dL, 3-3.5 g/dL, 3.5-5 g/dL, 3.5-4.5 g/dL, 3.5-4 g/dL, 4-5 g/dL, 4-4.5 g/dL, or 4.5-5 g/dL within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the increase of circulating hemoglobin level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the increase of circulating hemoglobin level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the blood loss condition is characterized based on a level of circulating ferritin. Ferritin is a blood protein that contains iron, the level of which indicates how much iron the body stores. In some embodiments, circulating ferritin levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment circulating ferritin levels in the subject. In some embodiments, the subject is slightly iron deficient due to the blood loss which manifests as a circulating ferritin level of 14-80 ng/ml. In some embodiments, prior to administration, a subject having the blood loss condition suitable for a hemojuvelin antagonist (e.g., anti-HJV antibody) is associated with a circulating ferritin levels of up to 5000 ng/ml. In some embodiments, prior to administration, a subject having the blood loss condition suitable for a hemojuvelin antagonist (e.g., anti-HJV antibody) is associated with a circulating ferritin level in the range of 10-5000 ng/ml, 50-4000 ng/ml, 100-3000 ng/ml, 200-2000 ng/ml, 300-1000 ng/ml, 500-750 ng/ml, 14-150 ng/ml, 14-140 ng/ml, 14-130 ng/ml, 14-120 ng/ml, 14-110 ng/ml, 14-100 ng/ml, 14-90 ng/ml, 14-80 ng/ml, 14-70 ng/ml, 14-60 ng/ml, 14-50 ng/ml, 14-40 ng/ml, 14-30 ng/ml, 14-20 ng/ml, 15-150 ng/ml, 15-140 ng/ml, 15-130 ng/ml, 15-120 ng/ml, 15-110 ng/ml, 15-100 ng/ml, 15-90 ng/ml, 15-80 ng/ml, 15-70 ng/ml, 15-60 ng/ml, 15-50 ng/ml, 15-40 ng/ml, 15-30 ng/ml, 15-20 ng/ml, 20-150 ng/ml, 20-140 ng/ml, 20-130 ng/ml, 20-120 ng/ml, 20-110 ng/ml, 20-100 ng/ml, 20-90 ng/ml, 20-80 ng/ml, 20-70 ng/ml, 20-60 ng/ml, 20-50 ng/ml, 20-40 ng/ml, 20-30 ng/ml, 20-25 ng/ml, 30-150 ng/ml, 30-140 ng/ml, 30-130 ng/ml, 30-120 ng/ml, 30-110 ng/ml, 30-100 ng/ml, 30-90 ng/ml, 30-80 ng/ml, 30-70 ng/ml, 30-60 ng/ml, 30-50 ng/ml, 30-40 ng/ml, 40-150 ng/ml, 40-140 ng/ml, 40-130 ng/ml, 40-120 ng/ml, 40-110 ng/ml, 40-100 ng/ml, 40-90 ng/ml, 40-80 ng/ml, 40-70 ng/ml, 40-60 ng/ml, 40-50 ng/ml, 50-150 ng/ml, 50-140 ng/ml, 50-130 ng/ml, 50-120 ng/ml, 50-110 ng/ml, 50-100 ng/ml, 50-90 ng/ml, 50-80 ng/ml, 50-70 ng/ml, 50-60 ng/ml, 60-150 ng/ml, 60-140 ng/ml, 60-130 ng/ml, 60-120 ng/ml, 60-110 ng/ml, 60-100 ng/ml, 60-90 ng/ml, 60-80 ng/ml, 60-70 ng/ml, 70-150 ng/ml, 70-140 ng/ml, 70-130 ng/ml, 70-120 ng/ml, 70-110 ng/ml, 70-100 ng/ml, 70-90 ng/ml, 70-80 ng/ml, 80-150 ng/ml, 80-140 ng/ml, 80-130 ng/ml, 80-120 ng/ml, 80-110 ng/ml, 80-100 ng/ml, 80-90 ng/ml, 90-150 ng/ml, 90-140 ng/ml, 90-130 ng/ml, 90-120 ng/ml, 90-110 ng/ml, 90-100 ng/ml, 100-150 ng/ml, 100-140 ng/ml, 100-130 ng/ml, 100-120 ng/ml, 100-110 ng/ml, 110-150 ng/ml, 110-140 ng/ml, 110-130 ng/ml, 110-120 ng/ml, 120-150 ng/ml, 120-140 ng/ml, 120-130 ng/ml, 140-150 ng/ml, 100-5000 ng/ml, 200-5000 ng/ml, 300-5000 ng/ml, 400-5000 ng/ml, 500-5000 ng/ml, 600-5000 ng/ml, 700-5000 ng/ml, 800-5000 ng/ml, 900-5000 ng/ml, 1000-5000 ng/ml, 1500-5000 ng/ml, 2000-5000 ng/ml, 2500-5000 ng/ml, 3000-5000 ng/ml, 3500-5000 ng/ml, 4000-5000 ng/ml, 4500-5000 ng/ml, 100-4000 ng/ml, 200-4000 ng/ml, 300-4000 ng/ml, 400-4000 ng/ml, 500-4000 ng/ml, 600-4000 ng/ml, 700-4000 ng/ml, 800-4000 ng/ml, 900-4000 ng/ml, 1000-4000 ng/ml, 1500-4000 ng/ml, 2000-4000 ng/ml, 2500-4000 ng/ml, 3000-4000 ng/ml, 3500-4000 ng/ml, 100-3000 ng/ml, 200-3000 ng/ml, 300-3000 ng/ml, 400-3000 ng/ml, 500-3000 ng/ml, 600-3000 ng/ml, 700-3000 ng/ml, 800-3000 ng/ml, 900-3000 ng/ml, 1000-3000 ng/ml, 1500-3000 ng/ml, 2000-3000 ng/ml, 2500-3000 ng/ml, 100-2000 ng/ml, 200-2000 ng/ml, 300-2000 ng/ml, 400-2000 ng/ml, 500-2000 ng/ml, 600-2000 ng/ml, 700-2000 ng/ml, 800-2000 ng/ml, 900-2000 ng/ml, 1000-2000 ng/ml, 1500-2000 ng/ml, 100-1000 ng/ml, 200-1000 ng/ml, 300-1000 ng/ml, 400-1000 ng/ml, 500-1000 ng/ml, 600-1000 ng/ml, 700-1000 ng/ml, 800-1000 ng/ml, or 900-1000 ng/ml. In some embodiments, prior to administration, a subject having the blood loss condition suitable for a hemojuvelin antagonist (e.g., anti-HJV antibody) is associated with a circulating ferritin level in the range of 14-150 ng/ml.

In some embodiments, after administration of an hemojuvelin antagonist (e.g., an anti-HJV antibody), the subject's circulating ferritin level decreases. In some embodiments, decrease of ferritin level indicates that the stored iron is released into circulation to be available for RBC production. In some embodiments, after administration of an hemojuvelin antagonist (e.g., an anti-HJV antibody), the subject's circulating ferritin level decreases by 10%-70%, 15%-70%, 20%-70%, 25%-70%, 30%-70%, 35%-70%, 40%-70%, 45%-70%, 50%-70%, 55%-70%, 60%-70%, 65%-70%, 10%-60%, 15%-60%, 20%-60%, 25%-60%, 30%-60%, 35%-60%, 40%-60%, 45%-60%, 50%-60%, 55%-60%, 10%-50%, 15%-50%, 20%-50%, 25%-50%, 30%-50%, 35%-50%, 40%-50%, 45%-50%, 10%-40%, 15%-40%, 20%-40%, 25%-40%, 30%-40%, 35%-40%, 10%-30%, 15%-30%, 20%-30%, 25%-30%, 10%-20%, or 15%-20% compared to circulating ferritin level prior to the administration. In some embodiments, after administration of an hemojuvelin antagonist (e.g., an anti-HJV antibody), the subject's circulating ferritin level decreases by 10%-70%, 15%-70%, 20%-70%, 25%-70%, 30%-70%, 35%-70%, 40%-70%, 45%-70%, 50%-70%, 55%-70%, 60%-70%, 65%-70%, 10%-60%, 15%-60%, 20%-60%, 25%-60%, 30%-60%, 35%-60%, 40%-60%, 45%-60%, 50%-60%, 55%-60%, 10%-50%, 15%-50%, 20%-50%, 25%-50%, 30%-50%, 35%-50%, 40%-50%, 45%-50%, 10%-40%, 15%-40%, 20%-40%, 25%-40%, 30%-40%, 35%-40%, 10%-30%, 15%-30%, 20%-30%, 25%-30%, 10%-20%, or 15%-20% within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the decrease of circulating ferritin level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the decrease of circulating ferritin level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the blood loss condition is characterized based on a level of serum iron. In some embodiments, serum iron levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment serum iron levels in the subject. In some embodiments, the subject is slightly iron deficient due to the blood loss which manifests as a serum iron level of at least 40 μg/dL. In some embodiments, the subject is slightly iron deficient due to the blood loss which manifests as a serum iron level in the range of 40-60 μg/dL. In some embodiments, prior to administration, a subject having the blood loss condition suitable for a hemojuvelin antagonist (e.g., anti-HJV antibody) is associated with a serum iron level in the range of 20-80 μg/dL, 20-70 μg/dL, 20-60 μg/dL, 20-50 μg/dL, 20-40 μg/dL, 20-30 μg/dL, 25-80 μg/dL, 25-70 μg/dL, 25-60 μg/dL, 25-50 μg/dL, 25-40 μg/dL, 25-30 μg/dL, 30-80 μg/dL, 30-70 μg/dL, 30-60 μg/dL, 30-50 μg/dL, 30-40 μg/dL, 35-80 μg/dL, 35-70 μg/dL, 35-60 μg/dL, 35-50 μg/dL, 35-40 μg/dL, 40-80 μg/dL, 40-70 μg/dL, 40-60 μg/dL, 40-50 μg/dL, 45-80 μg/dL, 45-70 μg/dL, 45-60 μg/dL, 45-50 μg/dL, 50-80 μg/dL, 50-70 μg/dL, 50-60 μg/dL, 60-80 μg/dL, 60-70 μg/dL, or 70-80 μg/dL.

In some embodiments, after administration of an hemojuvelin antagonist (e.g., an anti-HJV antibody), the subject's serum iron level increases. In some embodiments, increase of serum iron level indicates that more iron is available for RBC production. In some embodiments, after administration of an hemojuvelin antagonist (e.g., an anti-HJV antibody), the subject's serum iron level is in the range of 45-200 μg/dL, 45-180 μg/dL, 45-160 μg/dL, 45-140 μg/dL, 45-120 μg/dL, 45-100 μg/dL, 45-80 μg/dL, 45-60 μg/dL, 60-200 μg/dL, 60-180 μg/dL, 60-160 μg/dL, 60-140 μg/dL, 60-120 μg/dL, 60-100 μg/dL, 60-80 μg/dL, 70-200 μg/dL, 70-180 μg/dL, 70-160 μg/dL, 70-140 μg/dL, 70-120 μg/dL, 70-100 μg/dL, 70-80 μg/dL, 80-200 μg/dL, 80-180 μg/dL, 80-160 μg/dL, 80-140 μg/dL, 80-120 μg/dL, 80-100 μg/dL, 90-200 μg/dL, 90-180 μg/dL, 90-160 μg/dL, 90-140 μg/dL, 90-120 μg/dL, 90-100 μg/dL, 100-200 μg/dL, 100-180 μg/dL, 100-160 μg/dL, 100-140 μg/dL, 100-120 μg/dL, 120-200 μg/dL, 120-180 μg/dL, 120-160 μg/dL, 120-140 μg/dL, 140-200 μg/dL, 140-180 μg/dL, 140-160 μg/dL, 160-200 μg/dL, 160-180 μg/dL, 160-180 μg/dL, or 180-200 μg/dL. In some embodiments, after administration of an hemojuvelin antagonist (e.g., an anti-HJV antibody), the subject's serum iron level reaches the range of 45-200 μg/dL, 45-180 μg/dL, 45-160 μg/dL, 45-140 μg/dL, 45-120 μg/dL, 45-100 μg/dL, 45-80 μg/dL, 45-60 μg/dL, 60-200 μg/dL, 60-180 μg/dL, 60-160 μg/dL, 60-140 μg/dL, 60-120 μg/dL, 60-100 μg/dL, 60-80 μg/dL, 70-200 μg/dL, 70-180 μg/dL, 70-160 μg/dL, 70-140 μg/dL, 70-120 μg/dL, 70-100 μg/dL, 70-80 μg/dL, 80-200 μg/dL, 80-180 μg/dL, 80-160 μg/dL, 80-140 μg/dL, 80-120 μg/dL, 80-100 μg/dL, 90-200 μg/dL, 90-180 μg/dL, 90-160 μg/dL, 90-140 μg/dL, 90-120 μg/dL, 90-100 μg/dL, 100-200 μg/dL, 100-180 μg/dL, 100-160 μg/dL, 100-140 μg/dL, 100-120 μg/dL, 120-200 μg/dL, 120-180 μg/dL, 120-160 μg/dL, 120-140 μg/dL, 140-200 μg/dL, 140-180 μg/dL, 140-160 μg/dL, 160-200 μg/dL, 160-180 μg/dL, 160-180 μg/dL, or 180-200 μg/dL within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the increase of serum iron level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the increase of serum iron level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the blood loss condition is characterized based on a level of Reticulocytes Hemoglobin Content (RET-He or CHr). In some embodiments, CHr levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment CHr levels in the subject. In some embodiments, prior to administration, a subject having the blood loss condition suitable for a hemojuvelin antagonist (e.g., anti-HJV antibody) is associated with a CHr of in the range of 10-60 pg, 10-55 pg, 10-50 pg, 10-45 pg, 10-40 pg, 10-35 pg, 10-30 pg, 10-25 pg, 10-20 pg, 10-15 pg, 15-60 pg, 15-55 pg, 15-50 pg, 15-45 pg, 15-40 pg, 15-35 pg, 15-30 pg, 15-25 pg, 15-20 pg, 20-60 pg, 20-55 pg, 20-50 pg, 20-45 pg, 20-40 pg, 20-35 pg, 20-30 pg, 20-25 pg, 25-60 pg, 25-55 pg, 25-50 pg, 25-45 pg, 25-40 pg, 25-35 pg, 25-30 pg, 30-60 pg, 30-55 pg, 30-50 pg, 30-45 pg, 30-40 pg, 30-35 pg, 35-60 pg, 35-55 pg, 35-50 pg, 35-45 pg, 35-40 pg, 40-60 pg, 40-55 pg, 40-50 pg, 40-45 pg, 45-60 pg, 45-55 pg, 45-50 pg, 50-60 pg, 50-55 pg, or 50-60 pg. In some embodiments, a subject having the blood loss condition suitable for a hemojuvelin antagonist (e.g., anti-HJV antibody) is associated with a CHr of in the range of 20-40 pg.

In some embodiments, after administration of an hemojuvelin antagonist (e.g., an anti-HJV antibody), the subject's CHr level increases. In some embodiments, after administration of an hemojuvelin antagonist (e.g., an anti-HJV antibody), the subject's CHr level increases by 1%-10%, 1%-9%, 1%-8%, 1%-7%, 1%-6%, 1%-5%, 1%-4%, 1%-3%, 1%-2%, 2%-10%, 2%-9%, 2%-8%, 2%-7%, 2%-6%, 2%-5%, 2%-4%, 2%-3%, 3%-10%, 3%-9%, 3%-8%, 3%-7%, 3%-6%, 3%-5%, 3%-4%, 4%-10%, 4%-9%, 4%-8%, 4%-7%, 4%-6%, 4%-5%, 5%-10%, 5%-9%, 5%-8%, 5%-7%, 5%-6%, 6%-10%, 6%-9%, 6%-8%, 6%-7%, 7%-10%, 7%-9%, 7%-8%, 8%-10%, 8%-9%, or 9%-10% compared to CHr level prior to the administration. In some embodiments, after administration of an hemojuvelin antagonist (e.g., an anti-HJV antibody), the subject's CHr level increases by 1%-10%, 1%-9%, 1%-8%, 1%-7%, 1%-6%, 1%-5%, 1%-4%, 1%-3%, 1%-2%, 2%-10%, 2%-9%, 2%-8%, 2%-7%, 2%-6%, 2%-5%, 2%-4%, 2%-3%, 3%-10%, 3%-9%, 3%-8%, 3%-7%, 3%-6%, 3%-5%, 3%-4%, 4%-10%, 4%-9%, 4%-8%, 4%-7%, 4%-6%, 4%-5%, 5%-10%, 5%-9%, 5%-8%, 5%-7%, 5%-6%, 6%-10%, 6%-9%, 6%-8%, 6%-7%, 7%-10%, 7%-9%, 7%-8%, 8%-10%, 8%-9%, or 9%-10% within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the increase of the CHr level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the increase of CHr level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the blood loss condition is characterized based on a level of transferrin saturation (TSAT %). In some embodiments, TSAT % levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment TSAT % levels in the subject. In some embodiments, prior to administration, a subject having the blood loss condition suitable for a hemojuvelin antagonist (e.g., anti-HJV antibody) is associated with a TSAT % level in the range of 10%-40%, 10%-35%, 10%-30%, 10%-25%, 10%-20%, 10%-15%, 15%-40%, 15%-35%, 15%-30%, 15%-25%, 15%-20%, 20%-40%, 20%-35%, 20%-30%, 20%-25%, 25%-40%, 25%-35%, 25%-30%, 30%-40%, 30%-35%, 35%-40%. In some embodiments, a subject having the blood loss condition suitable for a hemojuvelin antagonist (e.g., anti-HJV antibody) is associated with a TSAT % in the range of 15%-30%.

In some embodiments, after administration of an hemojuvelin antagonist (e.g., an anti-HJV antibody), the subject's TSAT % level increases. In some embodiments, after administration of an hemojuvelin antagonist (e.g., an anti-HJV antibody), the subject's TSAT % level increases to the range of 25%-55%, 25%-50%, 25%-45%, 25%-40%, 25%-35%, 25%-30%, 30%-55%, 30%-50%, 30%-45%, 30%-40%, 30%-35%, 35%-55%, 35%-50%, 35%-45%, 35%-40%, 40%-55%, 40%-50%, 40%-45%, 45%-55%, 45%-50%, or 50%-55% compared to TSAT % level prior to the administration. In some embodiments, after administration of an hemojuvelin antagonist (e.g., an anti-HJV antibody), the subject's TSAT % level increases to the range of 25%-55%, 25%-50%, 25%-45%, 25%-40%, 25%-35%, 25%-30%, 30%-55%, 30%-50%, 30%-45%, 30%-40%, 30%-35%, 35%-55%, 35%-50%, 35%-45%, 35%-40%, 40%-55%, 40%-50%, 40%-45%, 45%-55%, 45%-50%, or 50%-55% within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the increase of the TSAT % level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the increase of TSAT % level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the blood loss condition is characterized based on a level of circulating hepcidin (e.g., hepcidin-25). In some embodiments, circulating hepcidin (e.g., hepcidin-25) levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment circulating hepcidin (e.g., hepcidin-25) levels in the subject. In some embodiments, prior to administration, a subject having the blood loss condition suitable for a hemojuvelin antagonist (e.g., anti-HJV antibody) is associated with a circulating hepcidin (e.g., hepcidin-25) level in the range of 2-100 ng/ml, 2-95 ng/ml, 2-90 ng/ml, 2-85 ng/ml, 2-80 ng/ml, 2-75 ng/ml, 2-70 ng/ml, 2-65 ng/ml, 2-60 ng/ml, 2-55 ng/ml, 2-50 ng/ml, 2-45 ng/ml, 2-40 ng/ml, 2-35 ng/ml, 2-30 ng/ml, 2-25 ng/ml, 2-20 ng/ml, 2-15 ng/ml, 2-10 ng/ml, 5-100 ng/ml, 5-95 ng/ml, 5-90 ng/ml, 5-85 ng/ml, 5-80 ng/ml, 5-75 ng/ml, 5-70 ng/ml, 5-65 ng/ml, 5-60 ng/ml, 5-55 ng/ml, 5-50 ng/ml, 5-45 ng/ml, 5-40 ng/ml, 5-35 ng/ml, 5-30 ng/ml, 5-25 ng/ml, 5-20 ng/ml, 5-15 ng/ml, 5-10 ng/ml, 10-100 ng/ml, 10-95 ng/ml, 10-90 ng/ml, 10-85 ng/ml, 10-80 ng/ml, 10-75 ng/ml, 10-70 ng/ml, 10-65 ng/ml, 10-60 ng/ml, 10-55 ng/ml, 10-50 ng/ml, 10-45 ng/ml, 10-40 ng/ml, 10-35 ng/ml, 10-30 ng/ml, 10-25 ng/ml, 10-20 ng/ml, 10-15 ng/ml, 20-100 ng/ml, 20-95 ng/ml, 20-90 ng/ml, 20-85 ng/ml, 20-80 ng/ml, 20-75 ng/ml, 20-70 ng/ml, 20-65 ng/ml, 20-60 ng/ml, 20-55 ng/ml, 20-50 ng/ml, 20-45 ng/ml, 20-40 ng/ml, 20-35 ng/ml, 20-30 ng/ml, 20-25 ng/ml, 30-100 ng/ml, 30-95 ng/ml, 30-90 ng/ml, 30-85 ng/ml, 30-80 ng/ml, 30-75 ng/ml, 30-70 ng/ml, 30-65 ng/ml, 30-60 ng/ml, 30-55 ng/ml, 30-50 ng/ml, 30-45 ng/ml, 30-40 ng/ml, 30-35 ng/ml, 40-100 ng/ml, 40-95 ng/ml, 40-90 ng/ml, 40-85 ng/ml, 40-80 ng/ml, 40-75 ng/ml, 40-70 ng/ml, 40-65 ng/ml, 40-60 ng/ml, 40-55 ng/ml, 40-50 ng/ml, 40-45 ng/ml, 50-100 ng/ml, 50-95 ng/ml, 50-90 ng/ml, 50-85 ng/ml, 50-80 ng/ml, 50-75 ng/ml, 50-70 ng/ml, 50-65 ng/ml, 50-60 ng/ml, 50-55 ng/ml, 60-100 ng/ml, 60-95 ng/ml, 60-90 ng/ml, 60-85 ng/ml, 60-80 ng/ml, 60-75 ng/ml, 60-70 ng/ml, 60-65 ng/ml, 70-100 ng/ml, 70-95 ng/ml, 70-90 ng/ml, 70-85 ng/ml, 70-80 ng/ml, 70-75 ng/ml, 80-100 ng/ml, 80-95 ng/ml, 80-90 ng/ml, 80-85 ng/ml, 90-100 ng/ml, 90-95 ng/ml, or 95-100 ng/ml. In some embodiments, prior to administration, a subject having the blood loss condition suitable for a hemojuvelin antagonist (e.g., anti-HJV antibody) is associated with a circulating hepcidin (e.g., hepcidin-25) level in the range of 5-75 ng/ml. In some embodiments, after administration of an hemojuvelin antagonist, the subject's circulating hepcidin (e.g., hepcidin-25) level decreases. In some embodiments, after administration of an hemojuvelin antagonist (e.g., an anti-HJV antibody), the subject's circulating hepcidin (e.g., hepcidin-25) level decreases by 2-60 ng/ml, 2-55 ng/ml, 2-50 ng/ml, 2-45 ng/ml, 2-40 ng/ml, 2-35 ng/ml, 2-30 ng/ml, 2-25 ng/ml, 2-20 ng/ml, 2-15 ng/ml, 2-10 ng/ml, 2-5 ng/ml, 5-60 ng/ml, 5-55 ng/ml, 5-50 ng/ml, 5-45 ng/ml, 5-40 ng/ml, 5-35 ng/ml, 5-30 ng/ml, 5-25 ng/ml, 5-20 ng/ml, 5-15 ng/ml, 5-10 ng/ml, 10-60 ng/ml, 10-55 ng/ml, 10-50 ng/ml, 10-45 ng/ml, 10-40 ng/ml, 10-35 ng/ml, 10-30 ng/ml, 10-25 ng/ml, 10-20 ng/ml, 10-15 ng/ml, 20-60 ng/ml, 20-55 ng/ml, 20-50 ng/ml, 20-45 ng/ml, 20-40 ng/ml, 20-35 ng/ml, 20-30 ng/ml, 20-25 ng/ml, 30-60 ng/ml, 30-55 ng/ml, 30-50 ng/ml, 30-45 ng/ml, 30-40 ng/ml, 30-35 ng/ml, 40-60 ng/ml, 40-55 ng/ml, 40-50 ng/ml, 40-45 ng/ml, 50-60 ng/ml, or 50-55 ng/ml compared to circulating hepcidin (e.g., hepcidin-25) level prior to the administration. In some embodiments, after administration of an hemojuvelin antagonist (e.g., an anti-HJV antibody), the subject's circulating hepcidin (e.g., hepcidin-25) level decreases by 2-60 ng/ml, 2-55 ng/ml, 2-50 ng/ml, 2-45 ng/ml, 2-40 ng/ml, 2-35 ng/ml, 2-30 ng/ml, 2-25 ng/ml, 2-20 ng/ml, 2-15 ng/ml, 2-10 ng/ml, 2-5 ng/ml, 5-60 ng/ml, 5-55 ng/ml, 5-50 ng/ml, 5-45 ng/ml, 5-40 ng/ml, 5-35 ng/ml, 5-30 ng/ml, 5-25 ng/ml, 5-20 ng/ml, 5-15 ng/ml, 5-10 ng/ml, 10-60 ng/ml, 10-55 ng/ml, 10-50 ng/ml, 10-45 ng/ml, 10-40 ng/ml, 10-35 ng/ml, 10-30 ng/ml, 10-25 ng/ml, 10-20 ng/ml, 10-15 ng/ml, 20-60 ng/ml, 20-55 ng/ml, 20-50 ng/ml, 20-45 ng/ml, 20-40 ng/ml, 20-35 ng/ml, 20-30 ng/ml, 20-25 ng/ml, 30-60 ng/ml, 30-55 ng/ml, 30-50 ng/ml, 30-45 ng/ml, 30-40 ng/ml, 30-35 ng/ml, 40-60 ng/ml, 40-55 ng/ml, 40-50 ng/ml, 40-45 ng/ml, 50-60 ng/ml, or 50-55 ng/ml within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the decrease of the circulating hepcidin (e.g., hepcidin-25) level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the decrease of the circulating hepcidin (e.g., hepcidin-25) level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HIV antibody).

In some embodiments, the blood loss condition is characterized based on a level of mean corpuscular hemoglobin (MCH) level. In some embodiments, MCH levels in a subject are determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment MCH levels in the subject. In some embodiments, prior to administration, a subject having the blood loss condition suitable for a hemojuvelin antagonist (e.g., anti-HJV antibody) is associated with a MCH level in the range of 15-50 pg, 15-45 pg, 15-40 pg, 15-35 pg, 15-30 pg, 15-25 pg, 15-20 pg, 20-50 pg, 20-45 pg, 20-40 pg, 20-35 pg, 20-30 pg, 20-25 pg, 25-50 pg, 25-45 pg, 25-40 pg, 25-35 pg, 25-30 pg, 30-50 pg, 30-45 pg, 30-40 pg, 30-35 pg, 35-50 pg, 35-45 pg, 35-40 pg, 40-50 pg, 40-45 pg, or 45-50 pg. In some embodiments, a subject having the blood loss condition suitable for a hemojuvelin antagonist (e.g., anti-HJV antibody) is associated with a MCH level in the range of 15-50 pg.

In some embodiments, after administration of an hemojuvelin antagonist (e.g., an anti-HJV antibody), the subject's MCH level increases. In some embodiments, after administration of an hemojuvelin antagonist (e.g., an anti-HJV antibody), the subject's MCH level increases by 1%-5%, 1%-4%, 1%-3%, 1%-2%, 2%-5%, 2%-4%, 2%-3%, 3%-5%, 3%-4%, or 4%-5% compared to MCH level prior to the administration. In some embodiments, after administration of an hemojuvelin antagonist (e.g., an anti-HJV antibody), the subject's MCH level increases by 1%-5%, 1%-4%, 1%-3%, 1%-2%, 2%-5%, 2%-4%, 2%-3%, 3%-5%, 3%-4%, or 4%-5% within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the increase of the MCH level in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the increase of the MCH level in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the blood loss condition is characterized based on the circulating red blood cell (RBC) count. In some embodiments, RBC count in a subject is determined (e.g., prior to treatment with a HJV antagonist (e.g., anti-HJV antibodies) or otherwise in absence of HJV antagonist (e.g., anti-HJV antibodies and compositions thereof) treatment at the time of determining) and compared to post-treatment RBC count in the subject. In some embodiments, prior to administration, a subject having the blood loss condition suitable for a hemojuvelin antagonist (e.g., anti-HJV antibody) is associated with a RBC count in the range of. 2×10¹² to 8×10¹² cells/L, 2×10¹² to 7×10¹² cells/L, 2×10¹² to 6×10¹² cells/L, 2×10¹² to 5×10¹² cells/L, 2×10¹² to 4×10¹² cells/L, 2×10¹² to 3×10¹² cells/L, 3×10¹² to 8×10¹² cells/L, 3×10¹² to 7×10¹² cells/L, 3×10¹² to 6×10¹² cells/L, 3×10¹² to 5×10¹² cells/L, 3×10¹² to 4×10¹² cells/L, 4×10¹² to 8×10¹² cells/L, 4×10¹² to 7×10¹² cells/L, 4×10¹² to 6×10¹² cells/L, 4×10¹² to 5×10¹² cells/L, 5×10¹² to 8×10¹² cells/L, 5×10¹² to 7×10¹² cells/L, 5×10¹² to 6×10¹² cells/L, 6×10¹² to 8×10¹² cells/L, or 7×10¹² to 8×10¹² cells/L. In some embodiments, a subject having the blood loss condition suitable for a hemojuvelin antagonist (e.g., anti-HJV antibody) is associated with an RBC count in the range of 3×10¹² to 6×10¹² cells/L.

In some embodiments, after administration of an hemojuvelin antagonist (e.g., an anti-HJV antibody), the subject's RBC count increases. In some embodiments, after administration of an hemojuvelin antagonist (e.g., an anti-HJV antibody), the subject's RBC count increases by at least 0.5×10¹² cells/L, at least 0.6×10¹² cells/L, at least 0.7×10¹² cells/L, at least 0.8×10¹² cells/L, at least 0.9×10¹² cells/L, at least 1.0×10¹² cells/L, at least 1.1×10¹² cells/L, at least 1.2×10¹² cells/L, at least 1.3×10¹² cells/L, at least 1.4×10¹² cells/L, at least 1.5×10¹² cells/L, at least 1.6×10¹² cells/L, at least 1.7×10¹² cells/L, at least 1.8×10¹² cells/L, at least 1.9×10¹² cells/L, at least 2.0×10¹² cells/L, at least 2.1×10¹² cells/L, at least 2.2×10¹² cells/L, at least 2.3×10¹² cells/L, at least 2.4×10¹² cells/L, or at least 2.5×10¹² cells/L compared to the RBC count prior to the administration. In some embodiments, after administration of an hemojuvelin antagonist (e.g., an anti-HJV antibody), the subject's RBC count increases by at least 0.5×10¹² cells/L, at least 0.6×10¹² cells/L, at least 0.7×10¹² cells/L, at least 0.8×10¹² cells/L, at least 0.9×10¹² cells/L, at least 1.0×10¹² cells/L, at least 1.1×10¹² cells/L, at least 1.2×10¹² cells/L, at least 1.3×10¹² cells/L, at least 1.4×10¹² cells/L, at least 1.5×10¹² cells/L, at least 1.6×10¹² cells/L, at least 1.7×10¹² cells/L, at least 1.8×10¹² cells/L, at least 1.9×10¹² cells/L, at least 2.0×10¹² cells/L, at least 2.1×10¹² cells/L, at least 2.2×10¹² cells/L, at least 2.3×10¹² cells/L, at least 2.4×10¹² cells/L, or at least 2.5×10¹² cells/L within one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, eight weeks, nine weeks, ten weeks, eleven weeks, twelve weeks, one month, two months, three months, four months, five month, or six months. In some embodiments, the increase of the RBC count in a subject receiving the hemojuvelin antagonist (e.g., anti-HJV antibody) is achieved within a shorter duration than would be achieved by a control subject who did not receive the hemojuvelin antagonist (e.g., anti-HJV antibody). In some embodiments, the increase of RBC count in a subject receiving the hemojuvelin antagonist (e.g., anti-HJV antibody) is achieved one day, two days, three days, four days, five days, six days, seven days, eight days, nine days, ten days, eleven days, thirteen days, fourteen days, fifteen days, sixteen days, seventeen days, eighteen days, nineteen days, twenty days, twenty-one days, twenty-two days, twenty-three days, twenty-four days, twenty-five days, twenty-six days, twenty-seven days, twenty-eight days, twenty-nine days, thirty days, one week, two weeks, three weeks, four weeks, five weeks, six weeks, seven weeks, or eight weeks faster than would be achieved by a control subject who did not receive the hemojuvelin antagonist (e.g., anti-HJV antibody).

In some embodiments, the subject does not have a functional iron deficiency (FID) prior to administration of a hemojuvelin antagonist (e.g., an anti-HJV antibody). FID represents a state of iron-restricted erythropoiesis characterized by an imbalance between iron demand and serum iron that is readily available for effective erythropoiesis. In FID, even when the body has adequate or increased systemic iron stores, iron is sequestered and not available for erythropoiesis. In some embodiments, a subject having a blood loss condition does not have functional iron deficiency prior to the hemojuvelin antagonist (e.g., an anti-HJV antibody) treatment. In some embodiments, upregulation of inflammatory cytokines in the bone marrow of subjects has also been associated with upregulation of circulating hepcidin, which in turn leads to anemia. In some embodiments, a subject does not have an anemia associated with inflammation prior to administration of a hemojuvelin antagonist (e.g., an anti-HJV antibody). In some embodiments, a subject has an anemia associated with inflammation prior to administration of a hemojuvelin antagonist (e.g., an anti-HJV antibody).

Determination of whether an amount of the HJV antagonist achieved the therapeutic effect would be evident to one of skill in the art based on the teachings provided herein. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. The particular dosage regimen, i.e., dose, timing and repetition, used in the method described herein will depend on the particular subject and that subject's medical history, as discussed herein.

Other aspects of the present disclosure relate to treating a subject having anemia, the method comprising administering to the subject an effective amount of anti-hemojuvelin antibody in monthly interval. In some embodiments, the anemia is associated with a functional iron deficiency. In some embodiments, the anemia is associated with inflammation.

Empirical considerations, such as time to maximum effect, half-life, and/or time above a specific concentration generally will contribute to the determination of the dosage of the hemojuvelin antagonist (e.g., anti-HJV antibody) to be used in the methods described herein (e.g., treating a subject having a blood loss condition or an anemia). In some embodiments, dosages for a HJV antagonist as described herein may be determined empirically in individuals who have been given one or more administration(s) of the HJV antagonist (e.g., anti-HJV antibody). Individuals are given incremental dosages of the hemojuvelin antagonist (e.g., anti-HJV antibody). To assess efficacy of the hemojuvelin antagonist (e.g., anti-HJV antibody), an indicator of the disease/disorder can be followed.

Dosing frequencies may vary in accordance with the methods described herein (e.g., treating a subject having a blood loss condition and/or anemia). In some embodiments, a hemojuvelin antagonist (e.g., an anti-HJV antibody) or a composition thereof is administered once. In some embodiments, a hemojuvelin antagonist (e.g., an anti-HJV antibody) or a composition thereof is administered on multiple occasions. In some embodiments, a hemojuvelin antagonist (e.g., an anti-HJV antibody) or a composition thereof is administered once monthly. In some embodiments, dosing frequency is every week, every 2 weeks, every 3 weeks, every 4 weeks, every 5 weeks, every 6 weeks, every 7 weeks, every 8 weeks, every 9 weeks, or every 10 weeks; or once every month, every 2 months, or every 3 months, or longer. In some embodiments, a hemojuvelin antagonist (e.g., an anti-HJV antibody) or a composition thereof is administered daily, biweekly, weekly, bimonthly, monthly, quarterly, or at any time interval that provide suitable (e.g., maximal) efficacy while minimizing safety risks to the subject. Generally, the efficacy and the treatment and safety risks may be monitored throughout the course of treatment.

In some embodiments, a subject may be administered a hemojuvelin antagonist (e.g., an anti-HJV antibody) or a composition thereof at one or more intervals during a set period of time. In some cases, periods of time during which a subject is administered a composition at one or more intervals may be separated by periods of time in which the subject is not administered the composition. In some embodiments, the relative durations of respective periods of time may depend on the subject's response to treatment or severity of disease or both and/or may be determined based on the judgment of a treating physician. For example, in some embodiments, during the course of a year a subject may be administered a composition weekly, biweekly or monthly for two months and then the administration is stopped for ten months. In some embodiments, during the course of a year a subject may be administered a composition weekly, biweekly or monthly for three months and then the administration is stopped for nine months. In some embodiments, during the course of a year a subject may be administered a composition weekly, biweekly or monthly for four months and then the administration is stopped for eight months. In some embodiments, during the course of a year a subject may be administered a composition weekly, biweekly or monthly for five months and then the administration is stopped for seven months. In some embodiments, during the course of a year a subject may be administered a composition weekly, biweekly or monthly for six months and then the administration is stopped for six months. In some embodiments, during the course of a year a subject may be administered a composition weekly, biweekly or monthly for seven months and then the administration is stopped for five months. In some embodiments, during the course of a year a subject may be administered a composition weekly, biweekly or monthly for eight months and then the administration is stopped for four months. In some embodiments, during the course of a year a subject may be administered a composition weekly, biweekly or monthly for nine months and then the administration is stopped for three months. In some embodiments, during the course of a year a subject may be administered a composition weekly, biweekly or monthly for ten months and then the administration is stopped for two months. In some embodiments, during the course of a year a subject may be administered a composition weekly, biweekly or monthly for two months on, two months off; or for three months on, three months off; or for four months on, four months off. In some embodiments, during the course of a year, a subject may be administered a composition quarterly for the entire duration of the year (i.e., four times). In some embodiments, during the course of a year, a subject may be administered a composition quarterly twice, or three times, but not four times.

In some embodiments, the hemojuvelin antagonist (e.g., an anti-HIV antibody) used in the methods (e.g., treating a subject having a blood loss condition and/or anemia) described herein is an anti-HJV antibody described herein. Generally, for administration of any of the anti-HJV antibodies described herein, a dose may be about 0.01 mg/kg, 0.05 mg/kg. 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, or 5 mg/kg.

In some embodiments, the dosage the anti-HJV antibody is up to 0.01 mg/kg, up to 0.05 mg/kg, up to 0.1 mg/kg, up to 0.2 mg/kg, up to 0.3 mg/kg, up to 0.4 mg/kg, up to 0.5 mg/kg, up to 0.6 mg/kg, up to 0.7 mg/kg, up to 0.8 mg/kg, up to 0.9 mg/kg, up to 1 mg/kg, mg/kg, up to 2 mg/kg, up to 3 mg/kg, up to 4 mg/kg, up to 5 g/kg or more.

In some embodiments, the dose of the anti-HJV antibody can be in a range of 0.01 mg/kg to 5 mg/kg, 0.01 mg/kg to 4 mg/kg, 0.01 mg/kg to 3 mg/kg, 0.01 mg/kg to 2 mg/kg, 0.01 mg/kg to 1 mg/kg, 0.01 mg/kg to 0.9 mg/kg, 0.01 mg/kg to 0.8 mg/kg, 0.01 mg/kg to 0.7 mg/kg, 0.01 mg/kg to 0.6 mg/kg, 0.01 mg/kg to 0.5 mg/kg, 0.01 mg/kg to 0.4 mg/kg, 0.01 mg/kg to 0.3 mg/kg, 0.01 mg/kg to 0.1 mg/kg, 0.01 mg/kg to 0.05 mg/kg, 0.01 mg/kg to 0.02 mg/kg, 0.05 mg/kg to 5 mg/kg, 0.05 mg/kg to 4 mg/kg, 0.05 mg/kg to 3 mg/kg, 0.05 mg/kg to 2 mg/kg, 0.05 mg/kg to 1 mg/kg, 0.05 mg/kg to 0.9 mg/kg, 0.05 mg/kg to 0.8 mg/kg, 0.05 mg/kg to 0.7 mg/kg, 0.05 mg/kg to 0.6 mg/kg, 0.05 mg/kg to 0.5 mg/kg, 0.05 mg/kg to 0.4 mg/kg, 0.05 mg/kg to 0.3 mg/kg, 0.05 mg/kg to 0.1 mg/kg, 0.1 mg/kg to 5 mg/kg, 0.1 mg/kg to 4 mg/kg, 0.1 mg/kg to 3 mg/kg, 0.1 mg/kg to 2 mg/kg, 0.1 mg/kg to 1 mg/kg, 0.1 mg/kg to 0.9 mg/kg, 0.1 mg/kg to 0.8 mg/kg, 0.1 mg/kg to 0.7 mg/kg, 0.1 mg/kg to 0.6 mg/kg, 0.1 mg/kg to 0.5 mg/kg, 0.1 mg/kg to 0.4 mg/kg, 0.1 mg/kg to 0.3 mg/kg, 0.5 mg/kg to 5 mg/kg, 0.5 mg/kg to 4 mg/kg, 0.5 mg/kg to 3 mg/kg, 0.5 mg/kg to 2 mg/kg, 0.5 mg/kg to 1 mg/kg, 0.5 mg/kg to 0.9 mg/kg, 0.5 mg/kg to 0.8 mg/kg, 0.5 mg/kg to 0.7 mg/kg, 0.5 mg/kg to 0.6 mg/kg, 1 mg/kg to 5 mg/kg, 1 mg/kg to 4 mg/kg, 1 mg/kg to 3 mg/kg, 1 mg/kg to 2 mg/kg, 2 mg/kg to 5 mg/kg, 2 mg/kg to 4 mg/kg, 2 mg/kg to 3 mg/kg, 3 mg/kg to 4 mg/kg, or 4 mg/kg to 5 mg/kg.

In some embodiments, the dose of the anti-HIV antibody can be in a range of 5-100 mg, 5-95 mg, 5-90 mg, 5-85 mg, 5-80 mg, 5-75 mg, 5-70 mg, 5-65 mg, 5-60 mg, 5-55 mg, 5-50 mg, 5-45 mg, 5-40 mg, 5-35 mg, 5-30 mg, 5-25 mg, 5-20 mg, 5-15 mg, 5-10 mg, 7-100 mg, 7-95 mg, 7-90 mg, 7-85 mg, 7-80 mg, 7-75 mg, 7-70 mg, 7-65 mg, 7-60 mg, 7-55 mg, 7-50 mg, 7-45 mg, 7-40 mg, 7-35 mg, 7-30 mg, 7-25 mg, 7-20 mg, 7-15 mg, 7-10 mg, 10-100 mg, 10-95 mg, 10-90 mg, 10-85 mg, 10-80 mg, 10-75 mg, 10-70 mg, 10-65 mg, 10-60 mg, 10-55 mg, 10-50 mg, 10-45 mg, 10-40 mg, 10-35 mg, 10-30 mg, 10-25 mg, 10-20 mg, 10-15 mg, 20-100 mg, 20-95 mg, 20-90 mg, 20-85 mg, 20-80 mg, 20-75 mg, 20-70 mg, 20-65 mg, 20-60 mg, 20-55 mg, 20-50 mg, 20-45 mg, 20-40 mg, 20-35 mg, 20-30 mg, 20-25 mg, 25-100 mg, 25-95 mg, 25-90 mg, 25-85 mg, 25-80 mg, 25-75 mg, 25-70 mg, 25-65 mg, 25-60 mg, 25-55 mg, 25-50 mg, 25-45 mg, 25-40 mg, 25-35 mg, 25-30 mg, 30-100 mg, 30-95 mg, 30-90 mg, 30-85 mg, 30-80 mg, 30-75 mg, 30-70 mg, 30-65 mg, 30-60 mg, 30-55 mg, 30-50 mg, 30-45 mg, 30-40 mg, 30-35 mg, 35-100 mg, 35-95 mg, 35-90 mg, 35-85 mg, 35-80 mg, 35-75 mg, 35-70 mg, 35-65 mg, 35-60 mg, 35-55 mg, 35-50 mg, 35-45 mg, 35-40 mg, 40-100 mg, 40-95 mg, 40-90 mg, 40-85 mg, 40-80 mg, 40-75 mg, 40-70 mg, 40-65 mg, 40-60 mg, 40-55 mg, 40-50 mg, 40-45 mg, 45-100 mg, 45-95 mg, 45-90 mg, 45-85 mg, 45-80 mg, 45-75 mg, 45-70 mg, 45-65 mg, 45-60 mg, 45-55 mg, 45-50 mg, 50-100 mg, 50-95 mg, 50-90 mg, 50-85 mg, 50-80 mg, 50-75 mg, 50-70 mg, 50-65 mg, 50-60 mg, 50-55 mg, 55-100 mg, 55-95 mg, 55-90 mg, 55-85 mg, 55-80 mg, 55-75 mg, 55-70 mg, 55-65 mg, 55-60 mg, 60-100 mg, 60-95 mg, 60-90 mg, 60-85 mg, 60-80 mg, 60-75 mg, 60-70 mg, 60-65 mg, 65-100 mg, 65-95 mg, 65-90 mg, 65-85 mg, 65-80 mg, 65-75 mg, 65-70 mg, 70-100 mg, 70-95 mg, 70-90 mg, 70-85 mg, 70-80 mg, 70-75 mg, 80-100 mg, 80-95 mg, 80-90 mg, 80-85 mg, 90-100 mg, 90-95 mg, or 95-100 mg. In some embodiments, the dose of the anti-HIV antibody can be in a range of 25-70 mg. In some embodiments, the dose of the anti-HJV antibody can be in a range of 35-65 mg. In some embodiments, the dose of the anti-HJV antibody can be in a range of 45-60 mg. In some embodiments, the dose of the anti-HJV antibody is 56 mg. In some embodiments, the dose of the anti-HJV antibody is 7 mg. In some embodiments, the dose of the anti-HJV antibody is 14 mg. In some embodiments, the dose of the anti-HJV antibody is 28 mg. In some embodiments, a subject is administered 56 mg of an anti-HJV antibody monthly.

In some embodiments, the antibodies described herein are administered to a subject in need of the treatment at an amount sufficient to inhibit the activity of the target antigen (e.g., an amount sufficient to inhibit HJV-induced BMP signaling) by at least 20% (e.g., 30%, 40%, 50%, 60%, 70%, 80%, 90% or greater) in vivo. In other embodiments, the antibody is administered in an amount effective in reducing the activity level of a target antigen by at least 20% (e.g., 30%, 40%, 50%, 60%, 70%, 80%, 90% or greater).

In some embodiments, an hemojuvelin antagonist (e.g., an anti-HJV antibody) can be administered parenterally. For example, a parenterally administered composition may be administered by subcutaneous, intracutaneous, intravenous, intraperitoneal, intratumor, intramuscular, intraarticular, intraarterial, or infusion techniques. In addition, it can be administered to the subject via injectable depot routes of administration such as using 1-, 3-, or 6-month depot injectable or biodegradable materials and methods.

In some embodiments, a hemojuvelin antagonist (e.g., an anti-HJV antibody) is administered intravenously. In some embodiments, a hemojuvelin antagonist (e.g., an anti-HJV antibody) is administered subcutaneously. In some embodiments, subcutaneous administration of a hemojuvelin antagonist (e.g., an anti-HJV antibody) results in similar bioavailability compared to intravenous administration of the same antibody at the same dose. In some embodiments, an anti-HJV antibody is administered subcutaneously at 56 mg. In some embodiments, an anti-HJV antibody is administered intravenously at 7 mg. In some embodiments, an anti-HJV antibody is administered subcutaneously at 14 mg. In some embodiments, an anti-HJV antibody is administered subcutaneously at 28 mg.

In some embodiments, subcutaneous administration of a hemojuvelin antagonist (e.g., an anti-HJV antibody) yields comparable pharmacodynamics effects (e.g., decreased circulating hepcidin-25 levels, increased TSAT %, increased CHr, decreased ferritin, increased MCH, increased RBC count and/or increased circulating hemoglobin) at lower maximum concentrations (Cmax) of the hemojuvelin antagonist (e.g., an anti-HJV antibody) compared to intravenous administration of the same antibody. Cmax is the maximum (or peak) serum concentration that a drug (e.g., a hemojuvelin antagonist (e.g., an anti-HJV antibody)) after the drug has been administered and before the administration of a second dose. In some embodiments, achieving a low Cmax within a short period of time (e.g., within 12 hours, within 24 hours, etc) after administration of a hemojuvelin antagonist (e.g., an anti-HJV antibody) minimizes undesirable increases in serum iron response, and/or minimizes chances of off-target effects of the hemojuvelin antagonist (e.g., an anti-HJV antibody) (e.g., binding to RGMa). In some embodiments, blunting Cmax by subcutaneous administration of a hemojuvelin antagonist (e.g., an anti-HJV antibody) avoids an undesirably sharp increase in serum iron response. In some embodiments, blunting Cmax by subcutaneous administration of a hemojuvelin antagonist (e.g., an anti-HJV antibody) reduces the off-target effects of the hemojuvelin antagonist (e.g., an anti-HJV antibody). In some embodiments, the Cmax reached by subcutaneous administration is at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than the Cmax reached by intravenous administration of a hemojuvelin antagonist (e.g., an anti-HJV antibody).

For intravenous injection, water soluble molecules (e.g., a hemojuvelin antagonist such as an anti-HJV antibody)) can be administered by the drip method, whereby a pharmaceutical formulation containing the molecule and a physiologically acceptable excipient is infused. Physiologically acceptable excipients may include, for example, 5% dextrose, 0.9% saline. Ringer's solution or other suitable excipients. Other injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). In some cases, preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, a hemojuvelin antagonist (e.g., an anti-HJV antibody) is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 2000/53211 and U.S. Pat. No. 5,981,568.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents. Treatment efficacy for a target disease/disorder can be assessed by methods well-known in the art.

The hemojuvelin antagonist (e.g., an anti-HJV antibody) and treatment methods involving such as described in the present disclosure may be utilized in combination with other types of therapy for the target disease or disorder disclosed herein. In this context, a hemojuvelin antagonist (e.g., an anti-HJV antibody) composition and a therapeutic agent may be given either simultaneously or sequentially. Examples include chemotherapy, immune therapy (e.g., therapies involving other HJV antagonists), surgery, radiation, gene therapy, and so forth, or anti-infection therapy. Such therapies can be administered simultaneously or sequentially (in any order) with the treatment according to the present disclosure.

In some embodiments, the combination therapy can include the hemojuvelin antagonist (e.g., an anti-HJV antibody) and pharmaceutical composition described herein, co-formulated with and/or co-administered with, at least one additional therapeutic agent described herein. Such combination therapies may advantageously utilize lower dosages of the administered therapeutic agents, thus preventing possible toxicities or complications associated with the various monotherapies. Moreover, the additional therapeutic agents disclosed herein may act on pathways in addition to or distinct from the hepcidin/BMP pathway, and thus may enhance and/or synergize with the effects of the hemojuvelin antagonist (e.g., an anti-HJV antibody).

In some embodiments, the combination therapy can include the hemojuvelin antagonist (e.g., an anti-HJV antibody) and pharmaceutical composition described herein, formulated in separate compositions with, at least one additional therapeutic agent described herein., and the different compositions can be administered simultaneously, or sequentially.

In some embodiments, the hemojuvelin antagonist (e.g., an anti-HJV antibody) is administered to a subject in combination with an erythropoietin stimulating agent. In some embodiments, the erythropoietin stimulating agent is selected from the group consisting of danazol, prednisone, thalidomide, lenalidomide, and pomalidomide.

In some embodiments, the hemojuvelin antagonist (e.g., an anti-HJV antibody) is administered to a subject in combination with an erythropoietin (EPO). Erythropoietin (EPO) is a glycoprotein hormone, naturally produced by the peritubular cells of the kidney, that stimulates red blood cell production. Recombinant EPOs have been previously described, for example, Epoetin alfa (Epogen/Procrit), Darbepoetin alfa (Aranesp), Methoxy polyethylene glycol-epoetin beta (Mircera), Epoetin alfa-epbx (Retacrit); or biosimilars to Epogen/Procrit.

In some embodiments, the hemojuvelin antagonist (e.g., an anti-HJV antibody) is administered to a subject in combination with a growth factor ligand trap. In some embodiments, the growth factor ligand trap is a transforming growth factor beta (TGF-β) ligand trap. In some embodiments, the TGF-β ligand trap is a GDF trap such as sotatercept or luspatercept. See U.S. Pat. No. 8,216,997, the contents of each of which are herein incorporated by reference.

In some embodiments, a subject in need of treatment in accordance with the disclosure continues to receive a therapeutic treatment for a underlying disease that causes the blood loss condition. The disclosure therefore provides, in some aspects, compositions and methods for treating blood loss condition and/or one or more conditions arising as a cause of the blood loss condition by administering to a subject in need thereof the hemojuvelin antagonist (e.g., an anti-HJV antibody) in combination with one or more therapeutic treatments for a disease that causes the blood loss condition as described herein.

In some embodiments, any of the disclosed hemojuvelin antagonists (e.g., an anti-HJV antibody) may be administered in a combination therapy with a therapeutic agent selected from an oral iron, IV iron, a HIF-PHI, and a red blood cell transfusion. In some embodiments, any of the disclosed hemojuvelin antagonists (e.g., an anti-HJV antibody) may be administered in combination with a diet the subject is on. For example, the subject may have a blood loss condition associated with celiac disease and is on a gluten-free diet while being administered with the hemojuvelin antagonist (e.g., an anti-HJV antibody).

In some embodiments, the hemojuvelin antagonist (e.g., an anti-HJV antibody) is administered to a subject in combination with an IV iron therapy, such as Iron Isomaltoside (MonoFerric®). Additional exemplary IV iron therapies include, but are not limited to, Iron Sucrose (Venofer®), Ferric Carboylmaltose (Ferrinject® or Injectofer®), Ferumoxytol (Ferraheme®), Iron Dextran (Imferon®), and sodium ferric gluconate in iron sucrose solution (Ferrlecit®).

In some embodiments, the hemojuvelin antagonist (e.g., an anti-HJV antibody) is administered to a subject in combination with an oral iron therapy. In some embodiments, the hemojuvelin antagonist (e.g., an anti-HJV antibody) provided herein may be combined with oral iron therapy to facilitate restoration of iron levels and/or treat anemic subjects who are experiencing intolerance to oral iron or an unsatisfactory response to oral iron. Examples of oral iron therapies include, but are not limited to, ferrous sulfate, ferric maltol (Accrufer®), ferrous gluconate, ferrous succinate, iron polymaltose, polysaccharide-iron complex, and ferrous sulfate. In other cases, iron may be administered intramuscularly (e.g., as iron sorbitol citrate).

In some embodiments, the hemojuvelin antagonist (e.g., an anti-HJV antibody) is administered to a subject in combination with an HIF-PHI, such as daprodustat, roxadustat and vadadustat. In some embodiments, the additional therapeutic agent is roxadustat.

### III. Hemojuvelin (HJV) Antagonists

In some aspects, hemojuvelin antagonists are provided herein to inhibit BMP-SMAD signaling for reducing expression and/or function of hepcidin, e.g., for modulating available iron for erythropoiesis for the treatment of a blood loss condition described herein in a subject in need thereof. In some embodiments, such methods are based on a recognition that increases in serum or tissue iron trigger transcriptional induction of hepcidin via the BMP-SMAD signaling pathway. In some embodiments, HJV serves as a BMP co-receptor to positively regulate hepcidin levels. In certain cells, e.g., hepatocytes, HJV-induced BMP signaling positively regulates hepcidin mRNA expression. In such embodiments, HJV binds to BMP2, BMP4, BMP5, and/or BMP6 to mediate BMP signaling, e.g., to positively regulate hepcidin levels in hepatocytes. In some embodiments, BMPs transduce signals by binding to one or a combination of type I and II serine/threonine kinase receptors. In some embodiments, upon ligand binding, constitutively active type II receptors phosphorylate type I receptors, and type I receptors then phosphorylate intracellular receptor-activated Smads (R-Smads), namely Smad 1, Smad 5 and/or Smad 8. In such embodiments, activated R-Smads complex with the common partner Smad4 and translocate to the nucleus to regulate gene transcription, e.g., induction of hepcidin expression.

In some embodiments, the hemojuvelin antagonist binds to one or more proteins of the repulsive guidance molecule (RGM) family, including RGMa, RGMb, and RGMc (HJV). In some embodiments, the hemojuvelin antagonist selectively binds hemojuvelin (RGMc) over RGMa and RGMb.

In some embodiments, the hemojuvelin antagonist binds to one or more proteins of the repulsive guidance molecule (RGM) family, including RGMa, RGMb, and RGMc (HJV). In some embodiments, the hemojuvelin antagonist selectively binds hemojuvelin (RGMc) over RGMa and RGMb. In some embodiments, the hemojuvelin antagonist is an antisense oligonucleotide that reduces expression of hemojuvelin (see, e.g., U.S. Pat. No. 7,534,764, entitled “Competitive regulation of hepcidin mRNA by soluble and cell-associated hemojuvelin,” issued May 19, 2009; US2014127325, entitled “Competitive regulation of hepcidin mRNA by soluble and cell-associated hemojuvelin”, issued May 19, 2009; and WO2016180784, entitled “Improved treatments using oligonucleotides”, published Nov. 17, 2016, which are incorporated herein by reference). In some embodiments, the hemojuvelin antagonist is a small molecule compound that inhibits hemojuvelin, e.g., by competitive binding and/or chemical modification of hemojuvelin.

In some embodiments, the HJV antagonist is a soluble HJV. In some embodiments, the soluble HJV is a soluble HJV-Fc fusion protein. In some embodiments, the soluble HJV is an soluble HJV as disclosed in U.S. Pat. No. 8,318,167B2, entitled “Methods and compositions for regulating iron homeostasis by modulation of BMP-6”. issued Nov. 27, 2012; U.S. Pat. No. 9,708,379B2, entitled “COMPOSITIONS FOR REGULATING IRON HOMEOSTASIS AND METHODS OF USING SAME,” issued Jul. 18, 2017, U.S. Pat. No. 10,273,273B2, entitled “COMPOSITIONS AND REGULATING IRON HOMEOSTASIS AND METHODS OF USING SAME,” issued Apr. 30, 2019, U.S. Pat. No. 7,968,091B2, entitled “METHODS AND COMPOSITIONS TO REGULATE IRON METABOLISM,” issued Jun. 28, 2011, U.S. Pat. No. 8,637,023B2, entitled “HEMOJUVELIN FUSION PROTEINS,” issued Jan. 28, 2014, U.S. Pat. No. 8,865,168B2, entitled “METHODS AND COMPOSITIONS TO REGULATE HEPCIDIN EXPRESSION,” issued Oct. 21, 2014, U.S. Pat. No. 9,556,251B2, entitled “METHODS AND COMPOSITIONS TO REGULATE HEPCIDIN EXPRESSION,” issued Jan. 31, 2017; U.S. Pat. No. 8,895,002B2, entitled “Hemojuvelin fusion proteins and uses thereof”, issued Nov. 25, 2014; U.S. Pat. No. 7,511,018B2, entitled “Juvenile hemochromatosis gene (HFE2A) cleavage products and uses thereof”, issued Mar. 31, 2009, the relevant contents of each of which are incorporated herein by reference. In some embodiments, the sHJV-Fc fusion protein is Ferruxmax. In some embodiments, the sHJV-Fc fusion protein is FMX-8.

In some embodiments, the hemojuvelin antagonist is an antibody specific for hemojuvelin and/or one or more proteins of the RGM protein family (e.g., RGMa, RGMb). In some embodiments, antibodies specific for hemojuvelin and/or one or more RGM proteins is an anti-HJV antibody and/or one or more RGM proteins as disclosed in U.S. Pat. No. 10,118,958, entitled “Composition and method for the diagnosis and treatment of iron-related disorders”, issued Nov. 6, 2018; U.S. Pat. No. 9,636,398, entitled “Composition and method for the diagnosis and treatment of iron-related disorders”, issued May 2, 2017; and U.S. Pat. No. 8,507,435, entitled “Juvenile hemochromatosis gene (HFE2A) cleavage products and uses thereof”, issued Aug. 13, 2013; U.S. Pat. No. 10,118,958, entitled “Composition and method for the diagnosis and treatment of iron-related disorders”, issued Nov. 6, 2018; US2010/0322941, entitled “Bone morphogenetic protein (BMP)-binding domains of proteins of the repulsive guidance molecule (RGM) protein family and functional fragments thereof, and use of same”, published Dec. 23, 2010; U.S. Pat. No. 9,040,052, entitled “Precision Medicine By Targeting Rare Human PCSK9 Variants for Cholesterol Treatment”, issued May 26, 2015; and US2017/0029499, entitled “Methods for treating hepcidin-mediated disorders”, published Feb. 2, 2017; and International Publication Nos. WO2007039256, entitled “Binding domains of proteins of the repulsive guidance molecule (rgm) protein family and functional fragments thereof, and their use,” published Apr. 12, 2007; WO2015171691, entitled “Compositions and methods for growth factor modulation”, published Nov. 12, 2015; WO2018/009624, entitled “Tgf-beta superfamily heteromultimers and uses thereof”, published Jan. 11, 2018, and WO2020/086736, entitled “Rgmc-selective inhibitors and use thereof”, published Apr. 30, 2020, the contents of each of which are incorporated herein by reference.

In some embodiments, the hemojuvelin antagonist is an antibody (e.g., hHA-001-hHA-012) specific for hemojuvelin and/or one or more proteins of the RGM protein family (e.g., RGMa, RGMb), as described herein in Table 1. Appropriate antibodies specific for hemojuvelin and/or one or more RGM proteins that may be useful in certain methods provided herein are provided for example, in U.S. Pat. Nos. 10,118,958; and 8,507,435; U.S. Patent Publication Nos. US 2013/330343; US 2015/166672; and US 2017/029499; and International Publication Nos. WO 2015/171691; and WO 2018/009624, which are incorporated herein by reference.

Provided herein, in some aspects, are antibodies that bind to human hemojuvelin (HJV) with high specificity and affinity. In some embodiments, the anti-HJV antibody described herein specifically binds to any extracellular epitope of a HJV or an epitope that becomes exposed to an antibody. In some embodiments, anti-HJV antibodies provided herein bind specifically to HJV from human, non-human primates, mouse, rat, etc. In some embodiments, anti-HJV antibodies provided herein bind to human HJV. In some embodiments, the anti-HJV antibody described herein binds to an amino acid segment of a human or non-human primate HJV.

In some embodiments, the anti-HIV antibody described herein specifically binds to an epitope on human HJV. Human HJV is a 426 amino acid protein with a predicted N-terminal signal peptide of 31 amino acids and a C-terminal GPI-attachment signal of 45 amino acids. An exemplary human HJV amino acid sequence is set forth in SEQ ID NO: 128:

(SEQ ID NO: 128)
MGEPGQSPSPRSSHGSPPTLSTLTLLLLLCGHAHSQCKILRCNAE
YVSSTLSLRGGGSSGALRGGGGGGRGGGVGSGGLCRALRSYALCT
RRTARTCRGDLAFHSAVHGIEDLMIQHNCSRQGPTAPPPPRGPAL
PGAGSGLPAPDPCDYEGRFSRLHGRPPGFLHCASFGDPHVRSFHH
HFHTCRVQGAWPLLDNDFLFVQATSSPMALGANATATRKLTIIFK
NMQECIDQKVYQAEVDNLPVAFEDGSINGGDRPGGSSLSIQTANP
GNHVEIQAAYIGTTIIIRQTAGQLSFSIKVAEDVAMAFSAEQDLQ
LCVGGCPPSQRLSRSERNRRGAITIDTARRLCKEGLPVEDAYFHS
CVFDVLISGDPNFTVAAQAALEDARAFLPDLEKLHLFPSDAGVPL
SSATLLAPLLSGLFVLWLCIQ

In some embodiments, the anti-HJV antibody described herein may bind to a fragment of a human HJV. The fragment of HJV may be between about 5 and about 425 amino acids, between about 10 and about 400 amino acids, between about 50 and about 350 amino acids, between about 100 and about 300 amino acids, between about 150 and about 250 amino acids, between about 200 and about 300 amino acids, or between about 75 and about 150 amino acids in length. The fragment may comprise a contiguous number of amino acids from RGMc. An exemplary amino acid of a HJV fragment is set forth in SEQ ID NO: 123:

(SEQ ID NO: 123)
QCKILRCNAEYVSSTLSLRGGGSSGALRGGGGGGRGGGVGSGGLC
RALRSYALCTRRTARTCRGDLAFHSAVHGIEDLMIQHNCSRQGPT
APPPPRGPALPGAGSGLPAPDPCDYEGRFSRLHGRPPGFLHCASF
GDPHVRSFHHHFHTCRVQGAWPLLDNDFLFVQATSSPMALGANAT
ATRKLTIIFKNMQECIDQKVYQAEVDNLPVAFEDGSINGGDRPGG
SSLSIQTANPGNHVEIQAAYIGTTIIIRQTAGQLSFSIKVAEDVA
MAFSAEQDLQLCVGGCPPSQRLSRSERNRRGAITIDTARRLCKEG
LPVEDAYFHSCVFDVLISGDPNFTVAAQAALEDARAFLPDLEKLH
LFPSD

In some embodiments, the anti-HJV antibody described herein binds to different epitopes within a human HJV or a human HJV fragment.

In some embodiments, the anti-HIV antibody interacts with an epitope within amino acids 160-190 of SEQ ID NO: 123. In some embodiments, the anti-HJV antibody interacts with an epitope having an amino acid sequence of amino acids 170-183 of SEQ ID NO: 123. In some embodiments, the anti-HJV antibody interacts with an epitope having the amino acid sequence of SSPMALGANATATR (SEQ ID NO: 121). In some embodiments, the anti-HJV antibody interacts with different segments within SSPMALGANATATR (SEQ ID NO: 121). In some embodiments, the anti-HJV antibody interacts with amino acids 170-171, amino acids 171-180, amino acids 180-182, and amino acids 182-183 of SEQ ID NO: 123. In some embodiments, the antibody interacts with amino acids 170 (S), 171 (S), 180 (T), 182 (T) and 183 (R) of SEQ ID NO: 123. In some embodiments, hHA-008 interacts with the epitope SSPMALGANATATR (SEQ ID NO: 121). In some embodiments, hHA-008 interacts with amino acids 170 (S), 171 (S), 180 (T), 182 (T) and 183 (R) of SEQ ID NO: 123.

In some embodiments, the anti-HJV antibody interacts with an epitope within amino acids 160-190 of SEQ ID NO: 123 and/or amino acids 280-310 of SEQ ID NO: 123. In some embodiments, the anti-HJV antibody interacts with an epitope within amino acids 169-182 of SEQ ID NO: 123 and/or amino acids 289-300 of SEQ ID NO: 123. In some embodiments, the anti-HJV antibody interacts with an epitope within amino acids 169-182 of SEQ ID NO: 123 and amino acids 289-300 of SEQ ID NO: 123. In some embodiments, the anti-HJV antibody interacts with an epitope having the amino acid sequence of TSSPMALGANATAT (SEQ ID NO: 122) and amino acid sequence SQRLSRSERNRR (SEQ ID NO: 127). In some embodiments, the anti-HJV antibody interacts with different segments within TSSPMALGANATAT (SEQ ID NO: 122) and SQRLSRSERNRR (SEQ ID NO: 127). In some embodiments, the anti-HJV antibody interacts with amino acids 169-171, amino acids 171-180, and amino acids 180-182 of SEQ ID NO: 123, and amino acids 289-293, amino acids 293-294, amino acids 294-295, amino acids 295-297 and amino acids 297-300 of SEQ ID NO: 123. In some embodiments, the antibody interacts with amino acids 169 (T), 170 (S), 171 (S), 180 (T), 182 (T), 289 (S), 293 (S), 294 (R), 295 (S), 297 (R), and 300 (R) of SEQ ID NO: 123. In some embodiments, hHA-008-QL interacts with different segments within TSSPMALGANATAT (SEQ ID NO: 122) and SQRLSRSERNRR (SEQ ID NO: 127). In some embodiments, hHA-008-QL interacts with amino acids 169 (T), 170 (S), 171 (S), 180 (T), 182 (T), 289 (S), 293 (S), 294 (R), 295 (S), 297 (R), and 300 (R) of SEQ ID NO: 123.

In some embodiments, the anti-HIV antibodies described herein are affinity matured clones. In some embodiments, an anti-HJV antibody specifically binds a HJV (e.g., a human or non-human primate HJV) with binding affinity (e.g., as indicated by K_{D}) of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. For example, the anti-HJV antibodies of the present disclosure can bind to a hemojuvelin protein (e.g., human hemojuvelin) with an affinity between 5 pM and 500 nM, e.g., between 50 pM and 100 nM, e.g., between 500 pM and 50 nM. The disclosure also includes antibodies that compete with any of the antibodies described herein for binding to a hemojuvelin protein (e.g., human hemojuvelin) and that have an affinity of 100 nM or lower (e.g., 80 nM or lower, 50 nM or lower, 20 nM or lower, 10 nM or lower, 500 pM or lower, 50 pM or lower, or 5 pM or lower). The affinity and binding kinetics of the anti-HJV antibody can be tested using any suitable method including but not limited to biosensor technology (e.g., OCTET or BIACORE). In some embodiments, the anti-HJV antibodies described herein binds to HJV with a K_{D} of sub-nanomolar range. In some embodiments, the anti-HJV antibodies described herein selectively binds to RGMc, but not RGMa or RGMb.

Binding affinity (or binding specificity) can be determined by a variety of methods including equilibrium dialysis, equilibrium binding, gel filtration, ELISA, surface plasmon resonance (SPR), florescent activated cell sorting (FACS) or spectroscopy (e.g., using a fluorescence assay). Exemplary conditions for evaluating binding affinity are in HBS-P buffer (10 mM HEPES pH7.4, 150 mM NaCl, 0.005% (v/v) surfactant P20) and PBS buffer (10 mM PO4-3, 137 mM NaCl, and 2.7 mM KCl). These techniques can be used to measure the concentration of bound proteins as a function of target protein concentration. The concentration of bound protein ([Bound]) is generally related to the concentration of free target protein ([Free]) by the following equation:

[Bound]=[Free]/(Kd+[Free])

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

The heavy chain (HC) and light chain (LC) sequences, heavy chain variable domain (VH) and light chain variable domain (VL), CDR sequences, and heavy chain and light chain constant region sequences of non-limiting examples of anti-HJV antibodies are provided in Table 1.

TABLE 1
Examples of anti-HJV antibodies (CDRs according to the Kabat definition)

| humanized Hemojuvelin | | | SEQ ID |
|---|---|---|---|
| Antagonist (HA) Antibody | | Sequences | NO |
| hHA-001 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLETSDGDTFLE | 4 |
| | LC CDR2 | EVSTRFS | 5 |
| | LC CDR3 | FQVTHDPMT | 6 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLETSDGDTFLEW | 8 |
| | | FQQRPGQSPRLLIYEVSTRFSGVPDRFSGSGSGTDFTLKI | |
| | | SRVEAEDVGVYYCFQVTHDPMTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKITPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLETSDGDTFLEW | 52 |
| | | FQQRPGQSPRLLIYEVSTRFSGVPDRFSGSGSGTDFTLKI | |
| | | SRVEAEDVGVYYCFQVTHDPMTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-002 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLETSDGDTFLE | 4 |
| | LC CDR2 | EVSSRFS | 49 |
| | LC CDR3 | MQVTHDPLT | 24 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLETSDGDTFLEW | 30 |
| | | FQQRPGQSPRLLIYEVSSRFSGVPDRFSGSGSGTDFTLKI | |
| | | SRVEAEDVGVYYCMQVTHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLIVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLETSDGDTFLEW | 53 |
| | | FQQRPGQSPRLLIYEVSSRFSGVPDRFSGSGSGTDFTLKI | |
| | | SRVEAEDVGVYYCMQVTHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSFNRGEC | |
| hHA-003 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLETSDGDTFLE | 4 |
| | LC CDR2 | EVSNRFS | 18 |
| | LC CDR3 | FQVTHDPVT | 25 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLETSDGDTFLEW | 31 |
| | | FQQRPGQSPRLLIYEVSNRFSGVPDRFSGSGSGTDFTLKI | |
| | | SRVEAEDVGVYYCFQVTHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSFNRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLETSDGDTFLEW | 54 |
| | | FQQRPGQSPRLLIYEVSNRFSGVPDRFSGSGSGTDFTLKI | |
| | | SRVEAEDVGVYYCFQVTHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSFNRGEC | |
| hHA-004 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLESSDGDTFLE | 14 |
| | LC CDR2 | DVSTRES | 19 |
| | LC CDR3 | FQVTHDPVT | 25 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLESSDGDTFLEW | 32 |
| | | FQQRPGQSPRLLIYDVSTRESGVPDRFSGSGSGTDFTLKI | |
| | | SRVEAEDVGVYYCFQVTHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLIVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLESSDGDTFLEW | 55 |
| | | FQQRPGQSPRLLIYDVSTRFSGVPDRFSGSGSGTDFTLKI | |
| | | SRVEAEDVGVYYCFQVTHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-005 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLEESDGYTFLE | 15 |
| | LC CDR2 | DVSERFS | 20 |
| | LC CDR3 | FQATYDPLT | 26 |
| | VH | EVQLVESGGGLVQPGGSLRLSCAASGFTFSNYGMNWIRQA | 7 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEESDGYTFLEW | 33 |
| | | FQQRPGQSPRLLIYDVSERFSGVPDRFSGSGSGTDFTLKI | |
| | | SRVEAEDVGVYYCFQATYDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSFNRGEC | |
| | Heavy Chain | EVQLVESGGGLVQPGGSLRLSCAASGFTFSNYGMNWIRQA | 51 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWIRQA | 114 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLEW | 56 |
| | | FQQRPGQSPRLLIYDVSERFSGVPDRFSGSGSGTDFTLKI | |
| | | SRVEAEDVGVYYCFQATYDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-006 | HC CDR1 | YYGMN | 9 |
| | HC CDR2 | MIYYDSSEKHYADSVKG | 2 |
| | HC CDR3 | GTTPDY | 3 |
| | LC CDR1 | RSSQSLEDSDGGTFLE | 16 |
| | LC CDR2 | DVSSRFS | 21 |
| | LC CDR3 | FQATHDPLT | 27 |
| | VH | EVQLVESGGGLVQPGGSLRLSCAASGFTFSYYGMNWIRQA | 34 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEDSDGGTFLEW | 35 |
| | | FQQRPGQSPRLLIYDVSSRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSYYGMNWIRQA | 57 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSYYGMNWIRQA | 115 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEDSDGGTFLEW | 58 |
| | | FQQRPGQSPRLLIYDVSSRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-007 | HC CDR1 | NYGMN | 1 |
| | HC CDR2 | MIYYDSSDKHYADSVKG | 10 |
| | HC CDR3 | GTTPDV | 11 |
| | LC CDR1 | RSSQSLEESDGYTFLH | 17 |
| | LC CDR2 | EVSNRFS | 18 |
| | LC CDR3 | FQATHDPVT | 28 |
| | VH | EVQLVESGGGLVQPGGSLRLSCAASGFTFSNYGMNWIRQA | 36 |
| | | PGKGLEWIGMIYYDSSDKHYADSVKGRFTISRDNSKNTLY | |
| | | LQMNSLRAEDTAVYYCAKGTTPDVWGQGTMVTVSS | |
| | VL | DVVLTQSPLSLPVTLGQPASISCRSSQSLEESDGYTFLHW | 37 |
| | | FQQRPGQSPRLLIYEVSNRFSGVPDRFSGSGSGTDFTLKI | |
| | | SRVEAEDVGVYYCFQATHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWIRQA | 59 |
| | (with or without | PGKGLEWIGMIYYDSSDKHYADSVKGRFTISRDNSKNTLY | |
| | | LQMNSLRAEDTAVYYCAKGTTPDVWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | the lysine residue | EVQLVESGGGLVQPGGSLRLSCAASGFTFSNYGMNWIRQA | 116 |
| | at the C-terminus | PGKGLEWIGMIYYDSSDKHYADSVKGRFTISRDNSKNTLY | |
| | of the heavy | LQMNSLRAEDTAVYYCAKGTTPDVWGQGTMVTVSSASTKG | |
| | chain) | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLHW | 60 |
| | | FQQRPGQSPRLLIYEVSNRFSGVPDRFSGSGSGTDFTLKI | |
| | | SRVEAEDVGVYYCFQATHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-008 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLEESDGYTFLH | 17 |
| | LC CDR2 | EVSTRFS | 5 |
| | LC CDR3 | FQATHDPLT | 27 |
| | VH | EVQLVESGGGLVQPGGSLRLSCAASGFTFSNYGMNWVRQA | 38 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEESDGYTFLHW | 39 |
| | | FQQRPGQSPRLLIYEVSTRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 61 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 117 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLHW | 62 |
| | | FQQRPGQSPRLLIYEVSTRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-008- | HC CDR1 | NYGMN | 1 |
| QL | HC CDR2 | MIYYDSSEKHYADSVKG | 2 |
| | HC CDR3 | GTTPDY | 3 |
| | LC CDR1 | RSSQSLEESDGYTFLH | 17 |
| | LC CDR2 | EVSTRFS | 5 |
| | LC CDR3 | FQATHDPLT | 27 |
| | VH | EVQLVESGGGLVQPGGSLRLSCAASGFTFSNYGMNWVRQA | 38 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLEESDGYTFLHW | 39 |
| | | FQQRPGQSPRLLIYEVSTRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 48 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDQLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVLHEALHNHYT | |
| | | QKSLSLSPGK | |
| | | ASTKGPSVFPLAPSSKSTSGGTAALGCLVKDYFPEPVTVS | 113 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDQLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVLHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 63 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDQLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVLHEALHNHYTQKSLS | |
| | | LSPGK | |
| | | EVQLVESGGGLVQPGGSLRLSCAASGFTFSNYGMNWVRQA | 118 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDQLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVLHEALHNHYTQKSLS | |
| | | LSPG | |
| | Light Chain | DVVLTQSPLSLPVTLGQPASISCRSSQSLEESDGYTFLHW | 62 |
| | | FQQRPGQSPRLLIYEVSTRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-009 | HC CDR1 | NYGMN | 1 |
| | HC CDR2 | MIYYDSSEKHYADSVKG | 2 |
| | HC CDR3 | GTTPDY | 3 |
| | LC CDR1 | RSSQSLADSDGDTFLH | 50 |
| | LC CDR2 | AVSHRFS | 22 |
| | LC CDR3 | FQATHDPVT | 28 |
| | VH | EVQLVESGGGLVQPGGSLRLSCAASGFTFSNYGMNWVRQA | 38 |
| | | PGKGLEWIGMIYYDSSEKHYADSVKGRFTISRDNSKNTLY | |
| | | LQMNSLRAEDTAVYYCAKGTTPDYWGQGTMVTVSS | |
| | VL | DVVLTQSPLSLPVTLGQPASISCRSSQSLADSDGDTFLHW | 41 |
| | | FQQRPGQSPRLLIYAVSHRFSGVPDRFSGSGSGTDFTLKI | |
| | | SRVEAEDVGVYYCFQATHDPVTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 61 |
| | (with or without | PGKGLEWIGMIYYDSSEKHYADSVKGRFTISRDNSKNTLY | |
| | the lysine residue | LQMNSLRAEDTAVYYCAKGTTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 117 |
| | | PGKGLEWIGMIYYDSSEKHYADSVKGRFTISRDNSKNTLY | |
| | | LQMNSLRAEDTAVYYCAKGTTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLADSDGDTFLHW | 65 |
| | | FQQRPGQSPRLLIYAVSHRFSGVPDRFSGSGSGTDFTLKI | |
| | | SRVEAEDVGVYYCFQATHDPVTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |
| hHA-010 | HC CDR1 | NYGMN | 1 |
| | HC CDR2 | MIYYDSSEKHYADSVKG | 2 |
| | HC CDR3 | GTTPDK | 12 |
| | LC CDR1 | RSSQSLEESDGYTFLE | 15 |
| | LC CDR2 | EVSHRFS | 23 |
| | LC CDR3 | FQATHDPLT | 27 |
| | VH | EVQLVESGGGLVQPGGSLRLSCAASGFTFSNYGMNWVRQA | 42 |
| | | PGKGLEWIGMIYYDSSEKHYADSVKGRFTISRDNSKNTLY | |
| | | LQMNSLRAEDTAVYYCAKGTTPDKWGQGTMVTVSS | |
| | VL | DVVLTQSPLSLPVTLGQPASISCRSSQSLEESDGYTFLEW | 43 |
| | | FQQRPGQSPRLLIYEVSHRFSGVPDRFSGSGSGTDFTLKI | |
| | | SRVEAEDVGVYYCFQATHDPLTFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 66 |
| | (with or without | PGKGLEWIGMIYYDSSEKHYADSVKGRFTISRDNSKNTLY | |
| | the lysine residue | LQMNSLRAEDTAVYYCAKGTTPDKWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 119 |
| | | PGKGLEWIGMIYYDSSEKHYADSVKGRFTISRDNSKNTLY | |
| | | LQMNSLRAEDTAVYYCAKGTTPDKWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVEL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEESDGYTFLEW | 67 |
| | | FQQRPGQSPRLLIYEVSHRFSGVPDRFSGSGSGTDFTLKI | |
| | | SRVEAEDVGVYYCFQATHDPLTFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSFNRGEC | |
| hHA-011 | HC CDR1 | NYGMN | 1 |
| | HC CDR2 | MIYYDSSEKHYADSVKG | 2 |
| | HC CDR3 | GSTPDY | 13 |
| | LC CDR1 | RSSQSLEDSDGGTFLE | 16 |
| | LC CDR2 | DVSSRFS | 21 |
| | LC CDR3 | FQATHDPLS | 29 |
| | VH | EVQLVESGGGLVQPGGSLRLSCAASGFTFSNYGMNWVRQA | 44 |
| | | PGKGLEWIGMIYYDSSEKHYADSVKGRFTISRDNSKNTLY | |
| | | LQMNSLRAEDTAVYYCAKGSTPDYWGQGTMVTVSS | |
| | VL | DVVLTQSPLSLPVTLGQPASISCRSSQSLEDSDGGTFLEW | 45 |
| | | FQQRPGQSPRLLIYDVSSRFSGVPDRFSGSGSGTDFTLKI | |
| | | SRVEAEDVGVYYCFQATHDPLSFGQGTKLEIK | |
| | HC Constant | ASTKGPSVFPLAPSSKSTSGGTAALGCLVKDYFPEPVTVS | 46 |
| | Region | WNSGALTSGVHTFPAVLQSSGLYSLSSVVTVPSSSLGTQT | |
| | (with or without | YICNVNHKPSNTKVDKKVEPKSCDKTHTCPPCPAPEAAGG | |
| | the lysine residue | PSVFLFPPKPKDTLMISRTPEVTCVVVDVSHEDPEVKENW | |
| | at the C-terminus | YVDGVEVHNAKTKPREEQYNSTYRVVSVLTVLHQDWLNGK | |
| | of the heavy | EYKCKVSNKALPAPIEKTISKAKGQPREPQVYTLPPSREE | |
| | chain constant | MTKNQVSLTCLVKGFYPSDIAVEWESNGQPENNYKTTPPV | |
| | region) | LDSDGSFFLYSKLTVDKSRWQQGNVFSCSVMHEALHNHYT | |
| | | QKSLSLSPGK | |
| | | ASTKGPSVFPLAPSSKSTSGGTAALGCLVKDYFPEPVTVS | 112 |
| | | WNSGALTSGVHTFPAVLQSSGLYSLSSVVTVPSSSLGTQT | |
| | | YICNVNHKPSNTKVDKKVEPKSCDKTHTCPPCPAPEAAGG | |
| | | PSVFLFPPKPKDTLMISRTPEVTCVVVDVSHEDPEVKENW | |
| | | YVDGVEVHNAKTKPREEQYNSTYRVVSVLTVLHQDWLNGK | |
| | | EYKCKVSNKALPAPIEKTISKAKGQPREPQVYTLPPSREE | |
| | | MTKNQVSLTCLVKGFYPSDIAVEWESNGQPENNYKTTPPV | |
| | | LDSDGSFFLYSKLTVDKSRWQQGNVFSCSVMHEALHNHYT | |
| | | QKSLSLSPG | |
| | LC Constant | RTVAAPSVFIFPPSDEQLKSGTASVVCLLNNFYPREAKVQ | 47 |
| | Region | WKVDNALQSGNSQESVTEQDSKDSTYSLSSTLTLSKADYE | |
| | | KHKVYACEVTHQGLSSPVTKSENRGEC | |
| | Heavy Chain | EVQLVESGGGLVQPGGSLRLSCAASGFTFSNYGMNWVRQA | 68 |
| | (with or without | PGKGLEWIGMIYYDSSEKHYADSVKGRFTISRDNSKNTLY | |
| | the lysine residue | LQMNSLRAEDTAVYYCAKGSTPDYWGQGTMVTVSSASTKG | |
| | at the C-terminus | PSVFPLAPSSKSTSGGTAALGCLVKDYFPEPVTVSWNSGA | |
| | of the heavy | LTSGVHTFPAVLQSSGLYSLSSVVTVPSSSLGTQTYICNV | |
| | chain) | NHKPSNTKVDKKVEPKSCDKTHTCPPCPAPEAAGGPSVEL | |
| | | FPPKPKDTLMISRTPEVTCVVVDVSHEDPEVKFNWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPGK | |
| | | EVQLVESGGGLVQPGGSLRLSCAASGFTFSNYGMNWVRQA | 120 |
| | | PGKGLEWIGMIYYDSSEKHYADSVKGRFTISRDNSKNTLY | |
| | | LQMNSLRAEDTAVYYCAKGSTPDYWGQGTMVTVSSASTKG | |
| | | PSVFPLAPSSKSTSGGTAALGCLVKDYFPEPVTVSWNSGA | |
| | | LTSGVHTFPAVLQSSGLYSLSSVVTVPSSSLGTQTYICNV | |
| | | NHKPSNTKVDKKVEPKSCDKTHTCPPCPAPEAAGGPSVFL | |
| | | FPPKPKDTLMISRTPEVTCVVVDVSHEDPEVKENWYVDGV | |
| | | EVHNAKTKPREEQYNSTYRVVSVLTVLHQDWLNGKEYKCK | |
| | | VSNKALPAPIEKTISKAKGQPREPQVYTLPPSREEMTKNQ | |
| | | VSLTCLVKGFYPSDIAVEWESNGQPENNYKTTPPVLDSDG | |
| | | SFFLYSKLTVDKSRWQQGNVFSCSVMHEALHNHYTQKSLS | |
| | | LSPG | |
| | Light Chain | DVVLTQSPLSLPVTLGQPASISCRSSQSLEDSDGGTFLEW | 69 |
| | | FQQRPGQSPRLLIYDVSSRFSGVPDRFSGSGSGTDFTLKI | |
| | | SRVEAEDVGVYYCFQATHDPLSFGQGTKLEIKRTVAAPSV | |
| | | FIFPPSDEQLKSGTASVVCLLNNFYPREAKVQWKVDNALQ | |
| | | SGNSQESVTEQDSKDSTYSLSSTLTLSKADYEKHKVYACE | |
| | | VTHQGLSSPVTKSENRGEC | |

In some embodiments, the N-terminus of the heavy chain of the anti-HJV antibody described herein is glutamic acid (E). In some embodiments, the glutamic acid can cyclize spontaneously to pyroglutamic acid by post-translational modification. Spontaneous cyclization of glutamic acid to pyroglutamic acid has been previously described, e.g., Chelius et al., Formation of Pyroglutamic Acid From N-terminal Glutamic Acid in Immunoglobulin Gamma Antibodies, Anal Chem. 2006; 78 (7): 2370-2376. In some embodiments, the N-terminus of the heavy chain of the anti-HJV antibody described herein is a pyroglutamic acid. In some embodiments, the anti-HJV antibodies having N-terminal pyroglutamic acid are impurities in the population of anti-HIV antibodies (e.g., less than 5%, less than 4%, less than 3%, less than 2%, less than 1%, less than 0.5%, less than 0.1%, less than 0.05%, or less than 0.01%) in the population of anti-HJV antibody. In some embodiments, the population of the anti-HJV antibodies comprises a mixture of anti-HJV antibodies having glutamic acid or pyroglutamic acid at the N-terminus of the heavy chain.

In some embodiments, the anti-HJV antibodies of the present disclosure comprise one or more of the HC CDRs (e.g., HC CDR1, HC CDR2, or HC CDR3) amino acid sequences from any one of the anti-HJV antibodies selected from Table 1. In some embodiments, the anti-HJV antibodies of the present disclosure comprise the HC CDR1, HC CDR2, and HC CDR3 as provided for any one of the antibodies elected from Table 1. In some embodiments, the anti-HJV antibodies of the present disclosure comprises one or more of the LC CDRs (e.g., LC CDR1, LC CDR2, or LC CDR3) amino acid sequences from any one of the anti-HIV antibodies selected from Table 1. In some embodiments, the anti-HIV antibodies of the present disclosure comprise the LC CDR1, LC CDR2, and LC CDR3 as provided for any one of the anti-HJV antibodies selected from Table 1.

In some embodiments, the anti-HJV antibodies of the present disclosure comprises the HC CDR1, HC CDR2, HC CDR3, LC CDR1, LC CDR2, and LC CDR3 as provided for any one of the anti-HJV antibodies selected from Table 1. In some embodiments, antibody heavy and light chain CDR3 domains may play a particularly important role in the binding specificity/affinity of an antibody for an antigen. Accordingly, the anti-HJV antibodies of the disclosure may include at least the heavy and/or light chain CDR3s of any one of the anti-HJV antibodies selected from Table 1.

In some embodiments, the isolated anti-HJV antibody comprises a heavy chain variable region that comprises a heavy chain CDR1 (HC CDR1), a heavy chain CDR2 (HC CDR2), and a heavy chain CDR3 (HC CDR3).

In some embodiments, following the Kabat definition, the HC CDR1 may comprise the amino acid sequence of X₁YGMN (SEQ ID NO: 105), in which X₁ can be N or Y. Alternatively or in addition, the HC CDR2 may comprise the amino acid sequence of MIYYDSSX₂KHYADSVKG (SEQ ID NO: 106), in which X₂ can be E or D. Alternatively or in addition, the HC CDR3 may comprise the amino acid sequence of GX₃TPDX₄ (SEQ ID NO: 107), in which X₃ can be T or S, and X₄ can be Y, V, or K.

In some embodiments, following the Kabat definition, the anti-HJV antibody may comprise a light chain variable region that comprises a light chain CDR1 (LC CDR1), a light chain CDR2 (LC CDR2), and a light chain CDR3 (LC CDR3). In some embodiments, the LC CDR1 may comprise the amino acid sequence of RSSQSLX₅X₆SDGX-TFLX₈ (SEQ ID NO: 108), in which X₅ can be A or E, X₆ can be T, S, E, or D, X₇ can be D, Y, or G, and X₈ can be E or H. Alternatively or in addition, the LC CDR2 may comprise the amino acid sequence of X₉VSX₁₀RFS (SEQ ID NO: 109), in which X₉ can be E, D or A, and X₁₀ can be N, S, T, E or H. Alternatively or in addition, the LC CDR3 may comprise the amino acid sequence of X₁₁QX₁₂TX₁₃DPX₁₄X₁₅ (SEQ ID NO: 110), in which X₁₁ can be F or M, X₁₂ can be V or A, X₁₃ can be H or Y, X₁₄ can be M, L or V, and X₁₅ can be T or S.

Also within the scope of the present disclosure are functional variants of any of the exemplary anti-HJV antibodies as disclosed herein. A functional variant may contain one or more amino acid residue variations in the V_{H} and/or V_{L}, or in one or more of the HC CDRs and/or one or more of the LC CDRs as relative to the reference antibody, while retaining substantially similar binding and biological activities (e.g., substantially similar binding affinity, binding specificity, inhibitory activity, anti-inflammatory activity, or a combination thereof) as the reference antibody.

In some embodiments, any of the anti-HJV antibodies of the disclosure have one or more CDRs (e.g., HC CDR or LC CDR) sequences substantially similar to any of the HC CDR1, HC CDR2, HC CDR3, LC CDR1, LC CDR2, and/or LC CDR3 sequences from one of the anti-HIV antibodies selected from Table 1. In some embodiments, the position of one or more CDRs along the VH (e.g., HC CDR1, HC CDR2, or HC CDR3) and/or VL (e.g., LC CDR1, LC CDR2, or LC CDR3) region of an antibody described herein can vary by one, two, three, four, five, or six amino acid positions so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived). For example, in some embodiments, the position defining a CDR of any antibody described herein can vary by shifting the N-terminal and/or C-terminal boundary of the CDR by one, two, three, four, five, or six amino acids, relative to the CDR position of any one of the antibodies described herein, so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived). In another embodiment, the length of one or more CDRs along the VH (e.g., HC CDR1, HC CDR2, or HC CDR3) and/or VL (e.g., LC CDR1, LC CDR2, or LC CDR3) region of an antibody described herein can vary (e.g., be shorter or longer) by one, two, three, four, five, or more amino acids, so long as immunospecific binding to hemojuvelin (e.g., human hemojuvelin) is maintained (e.g., substantially maintained, for example, at least 50%, at least 60%, at least 70%, at least 80%, at least 90%, at least 95% of the binding of the original antibody from which it is derived).

Accordingly, in some embodiments, a HC CDR1, HC CDR2, HC CDR3, LC CDR1, LC CDR2, and/or LC CDR3 described herein may be one, two, three, four, five or more amino acids shorter than one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, a HC CDR1, HC CDR2, HC CDR3, LC CDR1, LC CDR2, and/or LC CDR3 described herein may be one, two, three, four, five or more amino acids longer than one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be extended by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the amino portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, the carboxy portion of a HC CDR1, HC CDR2, HC CDR3, LC CDR1, LC CDR2, and/or LC CDR3 described herein can be shortened by one, two, three, four, five or more amino acids compared to one or more of the CDRs described herein (e.g., CDRS from any of the anti-HIV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). Any method can be used to ascertain whether immunospecific binding to hemojuvelin (e.g., human hemojuvelin) is maintained, for example, using binding assays and conditions described in the art.

In some examples, any of the anti-HJV antibodies of the disclosure have one or more CDR (e.g., HC CDR or LC CDR) sequences substantially similar to any one of the anti-HJV antibodies selected from Table 1. For example, the antibodies may include one or more CDR sequence(s) from any of the anti-HJV antibodies selected from Table 1 containing up to 5, 4, 3, 2, or 1 amino acid residue variations as compared to the corresponding CDR region in any one of the CDRs provided herein (e.g., CDRs from any of the anti-HJV antibodies selected from Table 1) so long as immunospecific binding to hemojuvelin (e.g., human hemojuvelin) is maintained (e.g., substantially maintained, for example, at least 50%, at least 60%, at least 70%, at least 80%, at least 90%, at least 95% relative to the binding of the original antibody from which it is derived). In some embodiments, any of the amino acid variations in any of the CDRs provided herein may be conservative variations. Conservative variations can be introduced into the CDRs at positions where the residues are not likely to be involved in interacting with a hemojuvelin protein (e.g., a human hemojuvelin protein), for example, as determined based on a crystal structure. Some aspects of the disclosure provide anti-HJV antibodies that comprise one or more of the heavy chain variable (VH) and/or light chain variable (VL) domains provided herein. In some embodiments, any of the VH domains provided herein include one or more of the HC CDR sequences (e.g., HC CDR1, HC CDR2, and HC CDR3) provided herein, for example, any of the CDR-H sequences provided in any one of the anti-HJV selected from Table 1. In some embodiments, any of the VL domains provided herein include one or more of the CDR-L sequences (e.g., LC CDR1, LC CDR2, and LC CDR3) provided herein, for example, any of the LC CDR sequences provided in any one of the anti-HJV antibodies selected from Table 1.

In some embodiments, the anti-HJV antibodies of the disclosure include any antibody that includes a heavy chain variable domain and/or a light chain variable domain of any one of the anti-HJV antibodies selected from Table 1, and variants thereof. In some embodiments, anti-HJV antibodies of the disclosure include any antibody that includes the heavy chain variable and light chain variable pairs of any anti-HJV antibodies selected from Table 1.

Aspects of the disclosure provide anti-HJV antibodies having a heavy chain variable (VH) and/or a light chain variable (VL) domain amino acid sequence homologous to any of those described herein. In some embodiments, the anti-HJV antibody comprises a heavy chain variable sequence or a light chain variable sequence that is at least 75% (e.g., 80%, 85%, 90%, 95%, 98%, or 99%) identical to the heavy chain variable sequence and/or any light chain variable sequence of any one of the anti-HJV antibodies selected from Table 1. In some embodiments, the homologous heavy chain variable and/or a light chain variable amino acid sequences do not vary within any of the CDR sequences provided herein. For example, in some embodiments, the degree of sequence variation (e.g., 75%, 80%, 85%, 90%, 95%, 98%, or 99%) may occur within a heavy chain variable and/or a light chain variable sequence excluding any of the CDR sequences provided herein. In some embodiments, any of the anti-HJV antibodies provided herein comprise a heavy chain variable sequence and a light chain variable sequence that comprises a framework sequence that is at least 75%, 80%, 85%, 90%, 95%, 98%, or 99% identical to the framework sequence of any anti-HJV antibodies selected from Table 1. In some embodiments, any of the anti-HJV antibodies provided herein comprise a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of any one of anti-HJV antibodies selected from Table 1 and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of any one of anti-HJV antibodies selected from Table 1.

In some embodiments, an anti-HJV antibody of the present disclosure is a humanized antibody (e.g., a humanized variant containing one or more CDRs of Table 1). In some embodiments, an anti-HJV antibody of the present disclosure comprises a HC CDR1, a HC CDR2, a HC CDR3, a LC CDR1, a LC CDR2, and a LC CDR3 that are the same as the HC CDR1, HC CDR2, HC CDR3, LC CDR1, LC CDR2, and LC CDR3 shown in Table 1, and comprises a humanized heavy chain variable region and/or a humanized light chain variable region. In some embodiments, a humanized heavy chain variable and/or a humanized light chain variable amino acid sequences do not vary within any of the CDR sequences provided herein. For example, in some embodiments, the degree of sequence variation (e.g., at least 75%, at least 80%, at least 85%, at least 90%, at least 91%, at least 92%, at least 93%, at least 94%, at least 95%, at least 96%, at least 97%, at least 98%, or at least 99%) may occur within a heavy chain variable and/or a light chain variable sequence excluding any of the CDR sequences provided herein. In some embodiments, a humanized anti-HJV antibody provided herein comprises a heavy chain variable sequence and a light chain variable sequence that comprises a framework sequence that is at least 75%, at least 80%, at least 85%, at least 90%, at least 91%, at least 92%, at least 93%, at least 94%, at least 95%, at least 96%, at least 97%, at least 98%, or at least 99% identical to the framework sequence of any anti-HJV antibodies selected from Table 1. In some embodiments, a humanized heavy chain variable and/or a humanized light chain variable amino acid sequences do not vary within any of the CDR sequences provided herein. For example, in some embodiments, the number of amino acid variations (e.g., no more than 20, 19, 18, 17, 16, 15, 14, 13, 12, 11, 10, 9, 8, 7, 6, 5, 4, 3, 2, or 1 amino acid variation) may occur within a heavy chain variable and/or a light chain variable sequence excluding any of the CDR sequences provided herein. In some embodiments, a humanized anti-HJV antibody provided herein comprises a heavy chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VH of any one of anti-HJV antibodies selected from Table 1 and/or a light chain variable sequence that comprises a framework sequence that that contains no more than 20, 19, 18, 17, 16, 15, 14, 13, 12, 11, 10, 9, 8, 7, 6, 5, 4, 3, 2, or 1 amino acid variation to the framework sequence of a VL of any one of anti-HJV antibodies selected from Table 1.

Humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some embodiments, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, six) which are altered with respect to the original antibody, which are also termed one or more CDRs derived from one or more CDRs from the original antibody. Humanized antibodies may also involve affinity maturation.

In some embodiments, humanization is achieved by grafting the CDRs (e.g., as shown in Table 1) into the human variable domains (e.g., IGKV1-NL1*01 and IGHV1-3*01 human variable domain). In some embodiments, the anti-HJV antibody of the present disclosure is a humanized variant comprising one or more amino acid substitutions (e.g., in the VH framework region) as compared with any one of the VHs listed in Table 1, and/or one or more amino acid substitutions (e.g., in the VL framework region) as compared with any one of the VLs listed in Table 1.

In some embodiments, the anti-HJV antibody of the present disclosure is a humanized antibody comprising a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH of any of the anti-HJV antibodies listed in Table 1. Alternatively or in addition, the anti-HJV antibody of the present disclosure is a humanized antibody comprising a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL of any one of the anti-HIV antibodies listed in Table 1.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 8.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 5, and a LC CDR3 having the amino acid sequence of SEQ ID NO: 6.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, the anti-HJV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 5, and LC CDR3 having the amino acid sequence of SEQ ID NO: 6.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 6.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 8.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 8.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 7. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 8.

In some embodiments, the anti-HIV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 30.

In some embodiments, according to the Kabat definition system, the anti-HIV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 49, and a LC CDR3 having the amino acid sequence of SEQ ID NO: 24.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 49, and LC CDR3 having the amino acid sequence of SEQ ID NO: 24.

In some embodiments, the anti-HJV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 49, and LC CDR3 having the amino acid sequence of SEQ ID NO: 24.

In some embodiments, the anti-HJV antibody of the present disclosure comprises: a HC CDR 1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 49; and/or a LC CDR3 having no more than 3 amino acid variations (e.g., no more than 3, 2, or 1 amino acid variation) as compared with the LC CDR3 having the amino acid sequence of SEQ ID NO: 24.

In some embodiments, the anti-HJV antibody of the present disclosure comprises a VH comprising the amino acid sequence of SEQ ID NO: 7. Alternatively or in addition, the anti-HJV antibody of the present disclosure comprises a V_{L} comprising the amino acid sequence of SEQ ID NO: 30.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 30.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 7. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 30.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 31.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 4, a LC CDR2 having the amino acid sequence of SEQ ID NO: 18, and a LC CDR3 having the amino acid sequence of SEQ ID NO: 25.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 4, LC CDR2 having the amino acid sequence of SEQ ID NO: 18, and LC CDR3 having the amino acid sequence of SEQ ID NO: 25.

In some embodiments, the anti-HJV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 4, LC CDR2 having the amino acid sequence of SEQ ID NO: 18, and LC CDR3 having the amino acid sequence of SEQ ID NO: 25.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HIV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 4; a LC CDR2 having no more than 3 amino acid variations (e.g., no more than 3, 2, or 1 amino acid variation) as compared with the LC CDR2 having the amino acid sequence of SEQ ID NO: 18; and/or a LC CDR3 having no more than 3 amino acid variations (e.g., no more than 3, 2, or 1 amino acid variation) as compared with the LC CDR3 having the amino acid sequence of SEQ ID NO: 25

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 31

In some embodiments, the anti-HIV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 31

In some embodiments, the anti-HIV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 7. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 31.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 32.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 14, a LC CDR2 having the amino acid sequence of SEQ ID NO: 19, and a LC CDR3 having the amino acid sequence of SEQ ID NO: 25.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HIV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 14, LC CDR2 having the amino acid sequence of SEQ ID NO: 19, and LC CDR3 having the amino acid sequence of SEQ ID NO: 25.

In some embodiments, the anti-HIV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 14, LC CDR2 having the amino acid sequence of SEQ ID NO: 19, and LC CDR3 having the amino acid sequence of SEQ ID NO: 25.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 14; a LC CDR2 having no more than 3 amino acid variations (e.g., no more than 3, 2, or 1 amino acid variation) as compared with the LC CDR2 having the amino acid sequence of SEQ ID NO: 19; and/or a LC CDR3 having no more than 3 amino acid variations (e.g., no more than 3, 2, or 1 amino acid variation) as compared with the LC CDR3 having the amino acid sequence of SEQ ID NO: 25.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 32.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 32.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 7. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 32.

In some embodiments, the anti-HJV antibody of the present disclosure comprises a HC CDR1, HC CDR2 and HC CDR3 of a heavy chain variable domain having the amino acid sequence of SEQ ID NO: 7. Alternatively or in addition, the anti-HJV antibody of the present disclosure comprises a LC CDR1, LC CDR2 and LC CDR3 of a light chain variable domain having the amino acid sequence of SEQ ID NO: 33.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 15, a LC CDR2 having the amino acid sequence of SEQ ID NO: 20, and a LC CDR3 having the amino acid sequence of SEQ ID NO: 26.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 15, LC CDR2 having the amino acid sequence of SEQ ID NO: 20, and LC CDR3 having the amino acid sequence of SEQ ID NO: 26.

In some embodiments, the anti-HIV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HIV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 15, LC CDR2 having the amino acid sequence of SEQ ID NO: 20, and LC CDR3 having the amino acid sequence of SEQ ID NO: 26.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 15; a LC CDR2 having no more than 3 amino acid variations (e.g., no more than 3, 2, or 1 amino acid variation) as compared with the LC CDR2 having the amino acid sequence of SEQ ID NO: 20; and/or a LC CDR3 having no more than 3 amino acid variations (e.g., no more than 3, 2, or 1 amino acid variation) as compared with the LC CDR3 having the amino acid sequence of SEQ ID NO: 26.

In some embodiments, the anti-HJV antibody of the present disclosure comprises a VH comprising the amino acid sequence of SEQ ID NO: 7. Alternatively or in addition, the anti-HJV antibody of the present disclosure comprises a VL comprising the amino acid sequence of SEQ ID NO: 33.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 7. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 33.

In some embodiments, the anti-HIV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 7. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 33.

In some embodiments, the anti-HIV antibody of the present disclosure comprises a HC CDR1, HC CDR2 and HC CDR3 of a heavy chain variable domain having the amino acid sequence of SEQ ID NO: 34. Alternatively or in addition, the anti-HJV antibody of the present disclosure comprises a LC CDR1, LC CDR2 and LC CDR3 of a light chain variable domain having the amino acid sequence of SEQ ID NO: 35.

In some embodiments, according to the Kabat definition system, the anti-HJV antibody of the present disclosure comprises a HC CDR1 having the amino acid sequence of SEQ ID NO: 9, a HC CDR2 having the amino acid sequence of SEQ ID NO: 2, a HC CDR3 having the amino acid sequence of SEQ ID NO: 3, a LC CDR1 having the amino acid sequence of SEQ ID NO: 16, a LC CDR2 having the amino acid sequence of SEQ ID NO: 21, and a LC CDR3 having the amino acid sequence of SEQ ID NO: 27.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 9, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 16, LC CDR2 having the amino acid sequence of SEQ ID NO: 21, and LC CDR3 having the amino acid sequence of SEQ ID NO: 27.

In some embodiments, the anti-HIV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 9, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 16, LC CDR2 having the amino acid sequence of SEQ ID NO: 21, and LC CDR3 having the amino acid sequence of SEQ ID NO: 27.

In some embodiments, the anti-HJV antibody of the present disclosure comprises: a HC CDR 1 having no more than 3 amino acid variations (e.g., no more than 3, 2, or 1 amino acid variation) as compared with the HC CDR1 having the amino acid sequence of SEQ ID NO: 9; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 16; a LC CDR2 having no more than 3 amino acid variations (e.g., no more than 3, 2, or 1 amino acid variation) as compared with the LC CDR2 having the amino acid sequence of SEQ ID NO: 21; and/or a LC CDR3 having no more than 3 amino acid variations (e.g., no more than 3, 2, or 1 amino acid variation) as compared with the LC CDR3 having the amino acid sequence of SEQ ID NO: 27.

In some embodiments, the anti-HJV antibody of the present disclosure comprises a VH comprising the amino acid sequence of SEQ ID NO: 34. Alternatively or in addition, the anti-HIV antibody of the present disclosure comprises a VL comprising the amino acid sequence of SEQ ID NO: 35.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 34. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 35.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 34. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 35.

In some embodiments, the anti-HJV antibody of the present disclosure comprises a HC CDR1, HC CDR2 and HC CDR3 of a heavy chain variable domain having the amino acid sequence of SEQ ID NO: 36. Alternatively or in addition, the anti-HJV antibody of the present disclosure comprises a LC CDR1, LC CDR2 and LC CDR3 of a light chain variable domain having the amino acid sequence of SEQ ID NO: 37.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 10, a HC CDR3 having the amino acid sequence of SEQ ID NO: 11, a LC CDR1 having the amino acid sequence of SEQ ID NO: 17, a LC CDR2 having the amino acid sequence of SEQ ID NO: 18, and a LC CDR3 having the amino acid sequence of SEQ ID NO: 28.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 10, and HC CDR3 having the amino acid sequence of SEQ ID NO: 11. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 17, LC CDR2 having the amino acid sequence of SEQ ID NO: 18, and LC CDR3 having the amino acid sequence of SEQ ID NO: 28.

In some embodiments, the anti-HJV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 10, and HC CDR3 having the amino acid sequence of SEQ ID NO: 11. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 17, LC CDR2 having the amino acid sequence of SEQ ID NO: 18, and LC CDR3 having the amino acid sequence of SEQ ID NO: 28.

In some embodiments, the anti-HIV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 10; and/or a HC CDR3 having no more than 3 amino acid variations (e.g., no more than 3, 2, or 1 amino acid variation) as compared with the HC CDR3 having the amino acid sequence of SEQ ID NO: 11. Alternatively or in addition, the anti-HIV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 17; a LC CDR2 having no more than 3 amino acid variations (e.g., no more than 3, 2, or 1 amino acid variation) as compared with the LC CDR2 having the amino acid sequence of SEQ ID NO: 18; and/or a LC CDR3 having no more than 3 amino acid variations (e.g., no more than 3, 2, or 1 amino acid variation) as compared with the LC CDR3 having the amino acid sequence of SEQ ID NO: 28.

In some embodiments, the anti-HJV antibody of the present disclosure comprises a VH comprising the amino acid sequence of SEQ ID NO: 36. Alternatively or in addition, the anti-HJV antibody of the present disclosure comprises a VL comprising the amino acid sequence of SEQ ID NO: 37.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 36. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 37.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 36. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 37.

In some embodiments, the anti-HJV antibody of the present disclosure comprises a HC CDR1, HC CDR2 and HC CDR3 of a heavy chain variable domain having the amino acid sequence of SEQ ID NO: 38. Alternatively or in addition, the anti-HJV antibody of the present disclosure comprises a LC CDR1, LC CDR2 and LC CDR3 of a light chain variable domain having the amino acid sequence of SEQ ID NO: 39.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 17, a LC CDR2 having the amino acid sequence of SEQ ID NO: 5, and a LC CDR3 having the amino acid sequence of SEQ ID NO: 27.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HIV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 17, LC CDR2 having the amino acid sequence of SEQ ID NO: 5, and LC CDR3 having the amino acid sequence of SEQ ID NO: 27.

In some embodiments, the anti-HJV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 17, LC CDR2 having the amino acid sequence of SEQ ID NO: 5, and LC CDR3 having the amino acid sequence of SEQ ID NO: 27.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 17; a LC CDR2 having no more than 3 amino acid variations (e.g., no more than 3, 2, or 1 amino acid variation) as compared with the LC CDR2 having the amino acid sequence of SEQ ID NO: 5; and/or a LC CDR3 having no more than 3 amino acid variations (e.g., no more than 3, 2, or 1 amino acid variation) as compared with the LC CDR3 having the amino acid sequence of SEQ ID NO: 27.

In some embodiments, the anti-HJV antibody of the present disclosure comprises a VH comprising the amino acid sequence of SEQ ID NO: 38. Alternatively or in addition, the anti-HJV antibody of the present disclosure comprises a VL comprising the amino acid sequence of SEQ ID NO: 39.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 38. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 39.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 38. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 39.

In some embodiments, the anti-HIV antibody of the present disclosure comprises a HC CDR1, HC CDR2 and HC CDR3 of a heavy chain variable domain having the amino acid sequence of SEQ ID NO: 38. Alternatively or in addition, the anti-HJV antibody of the present disclosure comprises a LC CDR1, LC CDR2 and LC CDR3 of a light chain variable domain having the amino acid sequence of SEQ ID NO: 41.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 3, a LC CDR1 having the amino acid sequence of SEQ ID NO: 50, a LC CDR2 having the amino acid sequence of SEQ ID NO: 22, and a LC CDR3 having the amino acid sequence of SEQ ID NO: 28.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 3. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 50, LC CDR2 having the amino acid sequence of SEQ ID NO: 22, and LC CDR3 having the amino acid sequence of SEQ ID NO: 28.

In some embodiments, the anti-HJV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 3. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 50, LC CDR2 having the amino acid sequence of SEQ ID NO: 22, and LC CDR3 having the amino acid sequence of SEQ ID NO: 28.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 3. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 50; a LC CDR2 having no more than 3 amino acid variations (e.g., no more than 3, 2, or 1 amino acid variation) as compared with the LC CDR2 having the amino acid sequence of SEQ ID NO: 22; and/or a LC CDR3 having no more than 3 amino acid variations (e.g., no more than 3, 2, or 1 amino acid variation) as compared with the LC CDR3 having the amino acid sequence of SEQ ID NO: 28.

In some embodiments, the anti-HIV antibody of the present disclosure comprises a VH comprising the amino acid sequence of SEQ ID NO: 38. Alternatively or in addition, the anti-HJV antibody of the present disclosure comprises a VL comprising the amino acid sequence of SEQ ID NO: 41.

In some embodiments, the anti-HIV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 38. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 41.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 38. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 41.

In some embodiments, the anti-HJV antibody of the present disclosure comprises a HC CDR1, HC CDR2 and HC CDR3 of a heavy chain variable domain having the amino acid sequence of SEQ ID NO: 42. Alternatively or in addition, the anti-HIV antibody of the present disclosure comprises a LC CDR1, LC CDR2 and LC CDR3 of a light chain variable domain having the amino acid sequence of SEQ ID NO: 43.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 12, a LC CDR1 having the amino acid sequence of SEQ ID NO: 15, a LC CDR2 having the amino acid sequence of SEQ ID NO: 23, and a LC CDR3 having the amino acid sequence of SEQ ID NO: 27.

In some embodiments, anti-HIV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 12. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 15, LC CDR2 having the amino acid sequence of SEQ ID NO: 23, and LC CDR3 having the amino acid sequence of SEQ ID NO: 27.

In some embodiments, the anti-HIV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 12. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 15, LC CDR2 having the amino acid sequence of SEQ ID NO: 23, and LC CDR3 having the amino acid sequence of SEQ ID NO: 27.

In some embodiments, the anti-HJV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 12. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 15; a LC CDR2 having no more than 3 amino acid variations (e.g., no more than 3, 2, or 1 amino acid variation) as compared with the LC CDR2 having the amino acid sequence of SEQ ID NO: 23; and/or a LC CDR3 having no more than 3 amino acid variations (e.g., no more than 3, 2, or 1 amino acid variation) as compared with the LC CDR3 having the amino acid sequence of SEQ ID NO: 27.

In some embodiments, the anti-HJV antibody of the present disclosure comprises a VH comprising the amino acid sequence of SEQ ID NO: 42. Alternatively or in addition, the anti-HJV antibody of the present disclosure comprises a VL comprising the amino acid sequence of SEQ ID NO: 43.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 42. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 43.

In some embodiments, the anti-HJV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 42. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 43.

In some embodiments, the anti-HJV antibody of the present disclosure comprises a HC CDR1, HC CDR2 and HC CDR3 of a heavy chain variable domain having the amino acid sequence of SEQ ID NO: 44. Alternatively or in addition, the anti-HJV antibody of the present disclosure comprises a LC CDR1, LC CDR2 and LC CDR3 of a light chain variable domain having the amino acid sequence of SEQ ID NO: 45.

In some embodiments, according to the Kabat definition system, the anti-HJV antibody of the present disclosure comprises a HC CDR1 having the amino acid sequence of SEQ ID NO: 1, a HC CDR2 having the amino acid sequence of SEQ ID NO: 2, a HC CDR3 having the amino acid sequence of SEQ ID NO: 13, a LC CDR1 having the amino acid sequence of SEQ ID NO: 16, a LC CDR2 having the amino acid sequence of SEQ ID NO: 21, and a LC CDR3 having the amino acid sequence of SEQ ID NO: 29.

In some embodiments, anti-HJV antibody of the present disclosure comprises a HC CDR1, a HC CDR2, and a HC CDR3, which collectively contains no more than 5 amino acid variations (e.g., no more than 5, 4, 3, 2, or 1 amino acid variation) as compared with the HC CDR1 having the amino acid sequence of SEQ ID NO: 1, HC CDR2 having the amino acid sequence of SEQ ID NO: 2, and HC CDR3 having the amino acid sequence of SEQ ID NO: 13. “Collectively,” as used anywhere in the present disclosure, means that the total number of amino acid variations in all of the three heavy chain CDRs is within the defined range. Alternatively or in addition, the anti-HJV antibody of the present disclosure comprises a LC CDR1, a LC CDR2, and a LC CDR3, which collectively contains no more than 5 amino acid variations (e.g., no more than 5, 4, 3, 2 or 1 amino acid variation) as compared with the LC CDR1 having the amino acid sequence of SEQ ID NO: 16, LC CDR2 having the amino acid sequence of SEQ ID NO: 21, and LC CDR3 having the amino acid sequence of SEQ ID NO: 29

In some embodiments, the anti-HIV antibody of the present disclosure comprises a HC CDR1, a HC CDR2, and a HC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the HC CDR1 having the amino acid sequence of SEQ ID NO: 1, HC CDR2 having the amino acid sequence of SEQ ID NO: 2, and HC CDR3 having the amino acid sequence of SEQ ID NO: 13. Alternatively or in addition, the anti-HJV antibody of the present disclosure comprises a LC CDR1, a LC CDR2, and a LC CDR3 that collectively are at least 80% (e.g., at least 80%, at least 85%, at least 90%, at least 91%, at least 92%, at least 93%, at least 94%, at least 95%, at least 96%, at least 97%, at least 98%, or at least 99%) identical to the to the LC CDR1 having the amino acid sequence of SEQ ID NO: 16, LC CDR2 having the amino acid sequence of SEQ ID NO: 21, and LC CDR3 having the amino acid sequence of SEQ ID NO: 29.

In some embodiments, the anti-HIV antibody of the present disclosure comprises: a HC CDR1 having no more than 3 amino acid variations (e.g., no more than 3, 2, or 1 amino acid variation) as compared with the HC CDR1 having the amino acid sequence of SEQ ID NO: 1; a HC CDR2 having no more than 3 amino acid variations (e.g., no more than 3, 2, or 1 amino acid variation) as compared with the HC CDR2 having the amino acid sequence of SEQ ID NO: 2; and/or a HC CDR3 having no more than 3 amino acid variations (e.g., no more than 3, 2, or 1 amino acid variation) as compared with the HC CDR3 having the amino acid sequence of SEQ ID NO: 13. Alternatively or in addition, the anti-HJV antibody of the present disclosure comprises: a LC CDR1 having no more than 3 amino acid variations (e.g., no more than 3, 2, or 1 amino acid variation) as compared with the LC CDR1 having the amino acid sequence of SEQ ID NO: 16; a LC CDR2 having no more than 3 amino acid variations (e.g., no more than 3, 2, or 1 amino acid variation) as compared with the LC CDR2 having the amino acid sequence of SEQ ID NO: 21; and/or a LC CDR3 having no more than 3 amino acid variations (e.g., no more than 3, 2, or 1 amino acid variation) as compared with the LC CDR3 having the amino acid sequence of SEQ ID NO: 29.

In some embodiments, the anti-HJV antibody of the present disclosure comprises a VH comprising the amino acid sequence of SEQ ID NO: 44. Alternatively or in addition, the anti-HJV antibody of the present disclosure comprises a VL comprising the amino acid sequence of SEQ ID NO: 45.

In some embodiments, the anti-HJV antibody of the present disclosure comprises a VH containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the VH as set forth in SEQ ID NO: 44. Alternatively or in addition, the anti-HJV antibody of the present disclosure comprises a VL containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the VL as set forth in SEQ ID NO: 45.

In some embodiments, the anti-HIV antibody of the present disclosure comprises a VH comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VH as set forth in SEQ ID NO: 44. Alternatively or in addition, the anti-HJV antibody of the present disclosure comprises a VL comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to the VL as set forth in SEQ ID NO: 45.

The CDRs of an antibody may have different amino acid sequences when different definition systems are used (e.g., the IMGT definition, the Kabat definition, or the Chothia definition). A definition system annotates each amino acid in a given antibody sequence (e.g., VH or VL sequence) with a number, and numbers corresponding to the heavy chain and light chain CDRs are provided in Table 2. The CDRs listed in Table 1 are defined in accordance with the Kabat definition. One skilled in the art is able to derive the CDR sequences using the different numbering systems for the anti-HJV antibodies provided in Table 1.

TABLE 2
CDR Definitions

| | IMGT¹ | Kabat² | Chothia³ |
|---|---|---|---|
| HC CDR1 | 27-38 | 31-35 | 26-32 |
| HC CDR2 | 56-65 | 50-65 | 53-55 |
| HC CDR3 | 105-116/117 | 95-102 | 96-101 |
| LC CDR1 | 27-38 | 24-34 | 26-32 |
| LC CDR2 | 56-65 | 50-56 | 50-52 |
| LC CDR3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

In some embodiments, the anti-HJV antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the anti-HIV antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, the anti-HJV antibody of the present disclosure comprises a VL domain and/or VH domain of any one of the anti-HJV antibodies selected from Table 1, and comprises a constant region comprising the amino acid sequences of the constant regions of an IgG, IgE, IgM, IgD, IgA or IgY immunoglobulin molecule, any class (e.g., IgG1, IgG2, IgG3, IgG4, IgA1 and IgA2), or any subclass (e.g., IgG2a and IgG2b) of immunoglobulin molecule. Non-limiting examples of human constant regions are described in the art, e.g., see Kabat E A et al., (1991) supra. An example of a human IgG1 constant region is given below:

(SEQ ID NO: 103)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKL
TVDKSRWQQGNVFSCSVMHEALHNHYTQKSLSLSPGK

In some embodiments, the heavy chain of any of the anti-HIV antibodies described herein comprises a mutant human IgG1 constant region. For example, the introduction of LALA mutations (a mutant derived from mAb b12 that has been mutated to replace the lower hinge residues Leu234 Leu235 with Ala234 and Ala235) in the CH2 domain of human IgG1 is known to reduce Fcg receptor binding (Bruhns, P., et al. (2009) and Xu, D. et al. (2000)). The mutant human IgG1 constant region is provided below (mutations bonded and underlined):

(SEQ ID NO: 46)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments, the heavy chain of any of the anti-HIV antibodies described herein further comprises mutations in human IgG1 constant region. For example, the introduction of T250Q and M248L substitutions. In some embodiments, such substitution may affect FcRn binding and serum half-life (WO2005047307 and WO2013063110). An exemplary IgG1 constant region comprising the LALA mutation and the QL mutation is provided below (mutations bonded and underlined):

(SEQ ID NO: 48)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDQLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVLHEALHNHYTQKSLSLSPGK

In some embodiments, during the production of the antibodies, particularly with Chinese Hamster Ovary Cells (CHO cells), it can be appreciated that the lysine at the C-terminus of the heavy chain is cleaved. Accordingly, a human IgG1 constant region within a secreted antibody can be:

(SEQ ID NO: 111)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

In some embodiments, a mutant human IgG1 comprising the LALA mutations in a secreted antibody can be:

(SEQ ID NO: 112)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPG

In some embodiments, a mutant human IgG1 comprising the LALA mutations and the QL mutations can be:

(SEQ ID NO: 113)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDQLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVLHEALHNHYTQKSLSLSPG

In some embodiments, the light chain of any of the anti-HIV antibodies described herein may further comprise a light chain constant region (CL), which can be any CL known in the art. In some examples, the CL is a kappa light chain. In other examples, the CL is a lambda light chain. In some embodiments, the CL is a kappa light chain, the sequence of which is provided below:

(SEQ ID NO: 47)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSENRGEC

Other antibody heavy and light chain constant regions are well known in the art, e.g., those provided in the IMGT database (www.imgt.org) or at www.vbase2.org/vbstat.php., both of which are incorporated by reference herein.

In some embodiments, the anti-HJV antibody described herein comprises a heavy chain comprising any one of the VH as listed in Table 1 or any variants thereof and a heavy chain constant region that is at least 80%, at least 85%, at least 90%, at least 95%, or at least 99% identical to SEQ ID NO: 46, SEQ ID NO: 48, SEQ ID NO: 112, or SEQ ID NO: 113. In some embodiments, the anti-HJV antibody described herein comprises a heavy chain comprising any one of the VH as listed in Table 1 or any variants thereof and a heavy chain constant region that contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 46, SEQ ID NO: 48, SEQ ID NO: 112, or SEQ ID NO: 113. In some embodiments, the anti-HJV antibody described herein comprises a heavy chain comprising any one of the VH as listed in Table 1 or any variants thereof and a heavy chain constant region as set forth in SEQ ID NO: 46. In some embodiments, the anti-HJV antibody described herein comprises heavy chain comprising any one of the VH as listed in Table 1 or any variants thereof and a heavy chain constant region as set forth in SEQ ID NO: 48. In some embodiments, the anti-HJV antibody described herein comprises heavy chain comprising any one of the VH as listed in Table 1 or any variants thereof and a heavy chain constant region as set forth in SEQ ID NO: 112. In some embodiments, the anti-HJV antibody described herein comprises heavy chain comprising any one of the VH as listed in Table 1 or any variants thereof and a heavy chain constant region as set forth in SEQ ID NO: 113.

In some embodiments, the anti-HJV antibody described herein comprises a light chain comprising any one of the VL as listed in Table 1 or any variants thereof and a light chain constant region that is at least 80%, at least 85%, at least 90%, at least 95%, or at least 99% identical to SEQ ID NO: 47. In some embodiments, the anti-HJV antibody described herein comprises a light chain comprising any one of the VL as listed in Table 1 or any variants thereof and a light chain constant region contains no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with SEQ ID NO: 47. In some embodiments, the anti-HJV antibody described herein comprises a light chain comprising any one of the VL as listed in Table 1 or any variants thereof and a light chain constant region set forth in SEQ ID NO: 47.

Examples of IgG heavy chain and light chain amino acid sequences of the anti-HJV antibodies described are provided in Table 1 above.

In some embodiments, the anti-HIV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51, 57, 59, 61, 63, 66, 68, 114, 115, 116, 117, 118, 119 or 120. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 52, 53, 54, 55, 56, 58, 60, 62, 65, 67 or 69. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 51, 57, 59, 61, 63, 66, 68, 114, 115, 116, 117, 118, 119 or 120. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 52, 53, 54, 55, 56, 58, 60, 62, 65, 67 or 69. In some embodiments, the anti-HJV antibody described herein comprises a heavy chain comprising the amino acid sequence of any one of SEQ ID NOs: 51, 57, 59, 61, 63, 66, 68, 114, 115, 116, 117, 118, 119 or 120. Alternatively or in addition, the anti-HJV antibody described herein comprises a light chain comprising the amino acid sequence of any one of SEQ ID NOs: 52, 53, 54, 55, 56, 58, 60, 62, 65, 67 or 69.

In some embodiments, the anti-HIV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 52. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 51 or 114. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 52. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 52.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 53. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 51 or 114. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 53. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 53.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 54. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 51 or 114. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 54. In some embodiments, the anti-HJV antibody described herein comprises a heavy chain comprising the amino acid sequence of any one of SEQ ID NOs: 51 or 114. Alternatively or in addition, the anti-HIV antibody described herein comprises a light chain comprising the amino acid sequence of any one of SEQ ID NOs: 54.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 55. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 51 or 114. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 55. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 55.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 51 or 114. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 56. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 51 or 114. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 56. In some embodiments, the anti-HJV antibody described herein comprises a heavy chain comprising the amino acid sequence of any one of SEQ ID NOs: 51 or 114. Alternatively or in addition, the anti-HJV antibody described herein comprises a light chain comprising the amino acid sequence of any one of SEQ ID NOs: 56.

In some embodiments, the anti-HIV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 57 or 115. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 58. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 57 or 115. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 58. In some embodiments, the anti-HJV antibody described herein comprises a heavy chain comprising the amino acid sequence of any one of SEQ ID NOs: 57 or 115. Alternatively or in addition, the anti-HJV antibody described herein comprises a light chain comprising the amino acid sequence of any one of SEQ ID NOs: 58.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 59 or 116. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 60. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 59 or 116. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 60. In some embodiments, the anti-HJV antibody described herein comprises a heavy chain comprising the amino acid sequence of any one of SEQ ID NOs: 59 or 116. Alternatively or in addition, the anti-HJV antibody described herein comprises a light chain comprising the amino acid sequence of any one of SEQ ID NOs: 60.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 61 or 117. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 62. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 61 or 117. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 62. In some embodiments, the anti-HJV antibody described herein comprises a heavy chain comprising the amino acid sequence of any one of SEQ ID NOs: 61 or 117. Alternatively or in addition, the anti-HJV antibody described herein comprises a light chain comprising the amino acid sequence of any one of SEQ ID NOs: 62.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 63 or 118. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 62. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 63 or 118. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 62. In some embodiments, the anti-HJV antibody described herein comprises a heavy chain comprising the amino acid sequence of any one of SEQ ID NOs: 63 or 118. Alternatively or in addition, the anti-HJV antibody described herein comprises a light chain comprising the amino acid sequence of any one of SEQ ID NOs: 62.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 61 or 117. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 65. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 61 or 117. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 65. In some embodiments, the anti-HJV antibody described herein comprises a heavy chain comprising the amino acid sequence of any one of SEQ ID NOs: 61 or 117. Alternatively or in addition, the anti-HJV antibody described herein comprises a light chain comprising the amino acid sequence of any one of SEQ ID NOs: 65.

In some embodiments, the anti-HIV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 66 or 119. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 67. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 66 or 119. Alternatively or in addition, the anti-HIV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 67. In some embodiments, the anti-HJV antibody described herein comprises a heavy chain comprising the amino acid sequence of any one of SEQ ID NOs: 66 or 119. Alternatively or in addition, the anti-HIV antibody described herein comprises a light chain comprising the amino acid sequence of any one of SEQ ID NOs: 67.

In some embodiments, the anti-HJV antibody of the present disclosure comprises a heavy chain containing no more than 20 amino acid variations (e.g., no more than 20, 19, 18, 17, 16, 15, 14, 13, 12, 11, 10, 9, 8, 7, 6, 5, 4, 3, 2, or 1 amino acid variation) as compared with the heavy chain as set forth in any one of SEQ ID NOs: 68 or 120. Alternatively or in addition, the anti-HJV antibody of the present disclosure comprises a light chain containing no more than 20 amino acid variations (e.g., no more than 20, 19, 18, 17, 16, 15, 14, 13, 12, 11, 9, 8, 7, 6, 5, 4, 3, 2, or 1 amino acid variation) as compared with the light chain as set forth in any one of SEQ ID NOs: 69. In some embodiments, the anti-HJV antibody described herein comprises a heavy chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 68 or 120. Alternatively or in addition, the anti-HJV antibody described herein comprises a light chain comprising an amino acid sequence that is at least 80% (e.g., at least 80%, at least 85%, at least 90%, at least 91%, at least 92%, at least 93%, at least 94%, at least 95%, at least 96%, at least 97%, at least 98%, or at least 99%) identical to any one of SEQ ID NOs: 69. In some embodiments, the anti-HJV antibody described herein comprises a heavy chain comprising the amino acid sequence of any one of SEQ ID NOs: 68 or 120. Alternatively or in addition, the anti-HJV antibody described herein comprises a light chain comprising the amino acid sequence of any one of SEQ ID NOs: 69.

The anti-HJV antibodies described herein can be in any antibody form, including, but not limited to, intact (i.e., full-length) antibodies, antigen-binding fragments thereof (such as Fab, F(ab′), F(ab′)2, Fv), single chain antibodies, bi-specific antibodies, or nanobodies. In some embodiments, the anti-HJV antibody described herein is a scFv. In some embodiments, the anti-HJV antibody described herein is a scFv-Fab (e.g., scFv fused to a portion of a constant region).

In some embodiments, conservative mutations can be introduced into antibody sequences (e.g., CDRs or framework sequences) at positions where the residues are not likely to be involved in interacting with a target antigen (e.g., hemojuvelin), for example, as determined based on a crystal structure. In some embodiments, one, two or more mutations (e.g., amino acid substitutions) are introduced into the Fc region of an anti-HJV antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of a muscle-targeting antibody described herein (e.g., in a CH2 domain (residues 231-340 of human IgG1) and/or CH3 domain (residues 341-447 of human IgG1) and/or the hinge region, with numbering according to the Kabat numbering system (e.g., the EU index in Kabat)) to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (i.e., substitutions, insertions or deletions) are introduced into an IgG constant domain, or FcRn-binding fragment thereof (e.g., an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid mutations (i.e., substitutions, insertions or deletions) are introduced into an IgG constant domain, or FcRn-binding fragment thereof (e.g., an Fc or hinge-Fc domain fragment) to decrease the half-life of the anti-HJV antibody in vivo. In some embodiments, one, two or more amino acid mutations (i.e., substitutions, insertions or deletions) are introduced into an IgG constant domain, or FcRn-binding fragment thereof (e.g., an Fc or hinge-Fc domain fragment) to increase the half-life of the antibody in vivo. In some embodiments, the antibodies can have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some embodiments, the constant region of the IgG1 of an antibody described herein comprises a methionine (M) to tyrosine (Y) substitution in position 252, a serine(S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281:23514-24). In some embodiments, an antibody comprises an IgG constant domain comprising one, two, three or more amino acid substitutions of amino acid residues at positions 251-257, 285-290, 308-314, 385-389, and 428-436, numbered according to the EU index as in Kabat.

In some embodiments, one, two or more amino acid substitutions are introduced into an IgG constant domain Fc region to alter the effector function(s) of the anti-HJV antibody. The effector ligand to which affinity is altered can be, for example, an Fc receptor or the C1 component of complement. This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody described herein to remove potential glycosylation sites on Fc region, which may reduce Fc receptor binding (see, e.g., Shields R L et al., (2001) J Biol Chem 276:6591-604).

In some embodiments, one or more amino in the constant region of an anti-HJV antibody described herein can be replaced with a different amino acid residue such that the antibody has altered C1q binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody described herein are altered to thereby alter the ability of the antibody to fix complement. This approach is described further in International Publication No. WO 94/29351. In some embodiments, the Fc region of an antibody described herein is modified to increase the ability of the antibody to mediate antibody dependent cellular cytotoxicity (ADCC) and/or to increase the affinity of the antibody for an Fcγ receptor. This approach is described further in International Publication No. WO 00/42072.

In some embodiments, the heavy and/or light chain variable domain(s) sequence(s) of the antibodies provided herein can be used to generate, for example, CDR-grafted, chimeric, humanized, or composite human antibodies or antigen-binding fragments, as described elsewhere herein. As understood by one of ordinary skill in the art, any variant, CDR-grafted, chimeric, humanized, or composite antibodies derived from any of the antibodies provided herein may be useful in the compositions and methods described herein and will maintain the ability to specifically bind hemojuvelin, such that the variant, CDR-grafted, chimeric, humanized, or composite antibody has at least 50%, at least 60%, at least 70%, at least 80%, at least 90%, at least 95% or more binding to hemojuvelin relative to the original antibody from which it is derived.

In some embodiments, the antibodies provided herein comprise mutations that confer desirable properties to the antibodies. For example, to avoid potential complications due to Fab-arm exchange, which is known to occur with native IgG4 mAbs, the antibodies provided herein may comprise a stabilizing ‘Adair’ mutation (Angal S., et al., “A single amino acid substitution abolishes the heterogeneity of chimeric mouse/human (IgG4) antibody,” Mol Immunol 30, 105-108; 1993), where serine 228 (EU numbering; residue 241 Kabat numbering) is converted to proline resulting in an IgG1-like hinge sequence. Accordingly, any of the antibodies may include a stabilizing ‘Adair’ mutation.

In some embodiments, an antibody is modified, e.g., modified via glycosylation, phosphorylation, sumoylation, and/or methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or phosphoglycosylation. In some embodiments, the one or more sugar or carbohydrate molecules are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g., a glycosyltransferase. In some embodiments, an antibody is functionalized with sugar or carbohydrate molecules as described in International Patent Application Publication WO2014065661, published on May 1, 2014, entitled, “Modified antibody, antibody-conjugate and process for the preparation thereof”.

In some embodiments, any one of the anti-HJV antibodies described herein may comprise a signal peptide in the heavy and/or light chain sequence (e.g., a N-terminal signal peptide). In some embodiments, the anti-HJV antibody described herein comprises any one of the VH and VL sequences, any one of the IgG heavy chain and light chain sequences, or any one of the F(ab′) heavy chain and light chain sequences described herein, and further comprises a signal peptide (e.g., a N-terminal signal peptide). In some embodiments, the signal peptide comprises the amino acid sequence of MEFGLSWLFLVAILKGVQC (SEQ ID NO: 104).

### IV. Preparation of the Anti-HJV Antibodies

Antibodies capable of binding hemojuvelin as described herein can be made by any method known in the art. See, for example, Harlow and Lane, (1998) Antibodies: A Laboratory Manual, Cold Spring Harbor Laboratory, New York.

In some embodiments, antibodies specific to a target antigen (e.g., HJV) can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are XenomouseRTM from Amgen, Inc. (Fremont, CA) and HuMAb-MouseRTM and TC MouseTM from Medarex, Inc. (Princeton, NJ) or H2L2 mice from Harbour Antibodies BV (Holland). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments. Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as an exemplary source of such DNA. Once isolated, the DNA may be placed into one or more expression vectors, which are then transfected into host cells such as E. coli cells, simian COS cells, Chinese hamster ovary (CHO) cells, human HEK293 cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions.

Alternatively, techniques described for the production of single chain antibodies (U.S. Pat. Nos. 4,946,778 and 4,704,692) can be adapted to produce a phage or yeast scFv library and scFv clones specific to HJV can be identified from the library following routine procedures. Positive clones can be subjected to further screening to identify those that has high HJV binding affinity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In one example, epitope mapping can be accomplished use H/D-Ex (hydrogen deuterium exchange) coupled with proteolysis and mass spectrometry. In an additional example, epitope mapping can be used to determine the sequence to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. Alternatively, competition assays can be performed using other antibodies known to bind to the same antigen to determine whether an antibody binds to the same epitope as the other antibodies. Competition assays are well known to those of skill in the art.

In some examples, an anti-HJV antibody is prepared by recombinant technology as exemplified below. Nucleic acids encoding the heavy and light chain of an anti-HJV antibody as described herein can be cloned into one expression vector, each nucleotide sequence being in operable linkage to a suitable promoter. In one example, each of the nucleotide sequences encoding the heavy chain and light chain is in operable linkage to a distinct promoter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV promoter, and the herpes simplex tk virus promoter.

Regulatable promoters can also be used. Such regulatable promoters include those using the lac repressor from E. coli as a transcription modulator to regulate transcription from lac operator bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-555115 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad, among others.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO bearing minimal promoter derived from the human cytomegalovirus (hCMV) promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art. Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies (e.g., the nucleic acid coding sequence listed in Table 3) may be introduced into suitable host cells for producing the antibodies. Non-limiting examples of the host cells include Chinese hamster ovary (CHO) cells, dhfr-CHO cell, human embryonic kidney (HEK)-293 cells, verda reno (VERO) cells, nonsecreting null (NSO) cells, human embryonic retinal (PER.C6) cells, Sp2/0 cells, baby hamster kidney (BHK) cells, Madin-Darby Canine Kidney (MDCK) cells, Madin-Darby Bovine Kidney (MDBK) cells, and monkey kidney CV1 line transformed by SV40 (COS) cells. In some embodiments, the host cell expressing the anti-HJV antibodies are CHO cells. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody. In some embodiments, the host cell comprises the nucleic acid encoding the heavy chain of the anti-HJV antibody. In some embodiments, the host cell comprises the nucleic acid encoding the light chain of the anti-HJV antibody. In some embodiments, the host cell comprises the nucleic acid encoding the heavy chain and the nucleic acid encoding the light chain.

In some embodiments, methods for preparing an antibody described herein involve a recombinant expression vector that encodes both the heavy chain and the light chain of an anti-HJV antibody, as also described herein. The recombinant expression vector can be introduced into a suitable host cell (e.g., a dhfr-CHO cell) by a conventional method, e.g., calcium phosphate mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-HJV antibody and the other encoding the light chain of the anti-HJV antibody. Both of the two recombinant expression vectors can be introduced into a suitable host cell (e.g., dhfr-CHO cell) by a conventional method, e.g., calcium phosphate-mediated transfection.

Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-HJV antibody as described herein (e.g., as provided in Table 3), vectors (e.g., expression vectors) containing such; and host cells comprising the vectors are within the scope of the present disclosure.

TABLE 3
Nucleic acids Sequences encoding the VH/VL of anti-HJV antibodies
listed in Table 1

| humanized HA | | Nucleic Acid Sequences | SEQ ID |
|---|---|---|---|
| Antibody | | | NO |
| hHA-001 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 72 |
| | | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAACGAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCACGACCCC | |
| | | ATGACGTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 73 |
| | Chain | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAACGAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCACGACCCC | |
| | | ATGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-002 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 74 |
| | | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTATGCAAGTCACCCACGACCCC | |
| | | CTGACCTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 75 |
| | Chain | ACCAGCCTCAATTTCATGCAGATCATCACAATCACTTGAGACGAGCGACG | |
| | | GAGACACTTTTCTTGAGTGGTTCCAACAAAGACCCGGACAAAGCCCACGC | |
| | | CTGCTTATTTACGAGGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTATGCAAGTCACCCACGACCCC | |
| | | CTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-003 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 76 |
| | | ACCAGCCTCAATTTCATGCAGATCATCACAATCACTTGAGACCAGCGACG | |
| | | GAGATACTTTTCTTGAGTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGTCACCCACGACCCC | |
| | | GTCACGTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 77 |
| | Chain | ACCAGCCTCAATTTCATGCAGATCATCACAATCACTTGAGACCAGCGACG | |
| | | GAGATACTTTTCTTGAGTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGTCACCCACGACCCC | |
| | | GTCACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-004 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 78 |
| | | ACCAGCCTCAATTTCATGCAGATCATCACAATCACTTGAGTCCAGCGACG | |
| | | GAGACACTTTTCTTGAGTGGTTCCAACAAAGACCCGGACAAAGCCCACGC | |
| | | CTGCTTATTTACGATGTATCAACTAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCATGACCCC | |
| | | GTGACCTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 79 |
| | Chain | ACCAGCCTCAATTTCATGCAGATCATCACAATCACTTGAGTCCAGCGACG | |
| | | GAGACACTTTTCTTGAGTGGTTCCAACAAAGACCCGGACAAAGCCCACGC | |
| | | CTGCTTATTTACGATGTATCAACTAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGTCACCCATGACCCC | |
| | | GTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-005 | VH | GAAGTACAGTTGGTAGAAAGCGGCGGAGGACTTGTGCAGCCAGGTGGATC | 70 |
| | | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGC | |
| | VL | GACGTAGTACTGACCCAAAGCCCCCTTTCTCTCCCAGTAACCCTCGGACA | 80 |
| | | ACCAGCCTCAATTTCATGCAGATCATCACAATCACTTGAGGAAAGCGACG | |
| | | GATACACTTTTCTTGAATGGTTCCAACAAAGACCCGGACAAAGCCCACGC | |
| | | CTGCTTATTTACGATGTATCAGAAAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCTACGACCCC | |
| | | CTCACCTTCGGACAAGGAACTAAGCTCGAAATCAAA | |
| | Heavy | GAAGTACAGTTGGTAGAAAGCGGCGGAGGACTTGTGCAGCCAGGTGGATC | 71 |
| | Chain | TCTCAGACTTTCATGTGCCGCAAGCGGTTTTACTTTTAGTAACTATGGAA | |
| | | TGAATTGGATTAGACAAGCGCCCGGAAAAGGATTGGAATGGATAGGAATG | |
| | | ATATACTACGATAGCTCAGAAAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGAACA | |
| | | ACACCAGATTATTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 81 |
| | Chain | ACCAGCCTCAATTTCATGCAGATCATCACAATCACTTGAGGAAAGCGACG | |
| | | GATACACTTTTCTTGAATGGTTCCAACAAAGACCCGGACAAAGCCCACGC | |
| | | CTGCTTATTTACGATGTATCAGAAAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCTACGACCCC | |
| | | CTCACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-006 | VH | GAAGTACAGTTGGTAGAAAGCGGCGGAGGACTTGTGCAGCCAGGTGGATC | 82 |
| | | TCTCAGACTTTCATGTGCCGCAAGCGGTTTTACTTTTAGTTACTATGGAA | |
| | | TGAACTGGATTAGACAAGCGCCCGGAAAAGGATTGGAATGGATAGGCATG | |
| | | ATATACTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 83 |
| | | ACCAGCCTCAATTTCATGCAGATCATCACAATCACTTGAGGACAGCGACG | |
| | | GAGGAACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGCGACCCACGACCCC | |
| | | CTGACGTTCGGACAAGGAACTAAGCTCGAAATCAAA | |
| | Heavy | GAAGTACAGTTGGTAGAAAGCGGCGGAGGACTTGTGCAGCCAGGTGGATC | 84 |
| | Chain | TCTCAGACTTTCATGTGCCGCAAGCGGTTTTACTTTTAGTTACTATGGAA | |
| | | TGAACTGGATTAGACAAGCGCCCGGAAAAGGATTGGAATGGATAGGCATG | |
| | | ATATACTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 85 |
| | Chain | ACCAGCCTCAATTTCATGCAGATCATCACAATCACTTGAGGACAGCGACG | |
| | | GAGGAACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTTCAAGCGACCCACGACCCC | |
| | | CTGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-007 | VH | GAAGTACAGTTGGTAGAAAGCGGCGGAGGACTTGTGCAGCCAGGTGGATC | 86 |
| | | TCTCAGACTTTCATGTGCCGCAAGCGGTTTTACTTTTAGTAACTATGGAA | |
| | | TGAACTGGATAAGACAAGCGCCCGGAAAAGGATTGGAATGGATAGGCATG | |
| | | ATATACTACGACAGCTCGGACAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCGGATGTATGGGGTCAAGGAACAATGGTAACCGTGTCAAGC | |
| | VL | GACGTAGTACTGACCCAAAGCCCCCTTTCTCTCCCAGTAACCCTCGGACA | 87 |
| | | ACCAGCCTCAATTTCATGCAGATCATCACAATCACTTGAAGAGAGCGACG | |
| | | GATACACTTTTCTTCATTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | GTGACGTTCGGACAAGGAACTAAGCTCGAAATCAAA | |
| | Heavy | GAAGTACAGTTGGTAGAAAGCGGCGGAGGACTTGTGCAGCCAGGTGGATC | 88 |
| | Chain | TCTCAGACTTTCATGTGCCGCAAGCGGTTTTACTTTTAGTAACTATGGAA | |
| | | TGAACTGGATAAGACAAGCGCCCGGAAAAGGATTGGAATGGATAGGCATG | |
| | | ATATACTACGACAGCTCGGACAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACG | |
| | | ACGCCGGATGTATGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 89 |
| | Chain | ACCAGCCTCAATTTCATGCAGATCATCACAATCACTTGAAGAGAGCGACG | |
| | | GATACACTTTTCTTCATTGGTTCCAACAAAGACCCGGACAAAGCCCACGC | |
| | | CTGCTTATTTACGAGGTATCAAACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | GTGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-008 | VH | GAAGTACAGTTGGTAGAAAGCGGCGGAGGACTTGTGCAACCAGGTGGATC | 90 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 91 |
| | | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAA | |
| | Heavy | GAAGTACAGTTGGTAGAAAGCGGCGGAGGACTTGTGCAACCAGGTGGATC | 92 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 93 |
| | Chain | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-008- | VH | GAAGTACAGTTGGTAGAAAGCGGCGGAGGACTTGTGCAACCAGGTGGATC | 90 |
| QL | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 91 |
| | | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAA | |
| | Heavy | GAAGTACAGTTGGTAGAAAGCGGCGGAGGACTTGTGCAACCAGGTGGATC | 94 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACCAACTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGCTGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 93 |
| | Chain | ACCAGCCTCAATTTCATGCAGATCATCACAATCACTTGAAGAAAGCGACG | |
| | | GATACACTTTTCTTCACTGGTTCCAACAAAGACCCGGACAAAGCCCACGC | |
| | | CTGCTTATTTACGAGGTATCAACCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTGACCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-009 | VH | GAAGTACAGTTGGTAGAAAGCGGCGGAGGACTTGTGCAACCAGGTGGATC | 90 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 95 |
| | | ACCAGCCTCAATTTCATGCAGATCATCACAATCACTTGCGGACAGCGACG | |
| | | GAGATACTTTTCTTCACTGGTTCCAACAAAGACCCGGACAAAGCCCACGC | |
| | | CTGCTTATTTACGCGGTATCACACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCATGACCCC | |
| | | GTCACGTTCGGACAAGGAACTAAGCTCGAAATCAAA | |
| | Heavy | GAAGTACAGTTGGTAGAAAGCGGCGGAGGACTTGTGCAACCAGGTGGATC | 92 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATATTACGACAGCTCGGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGGACC | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 96 |
| | Chain | ACCAGCCTCAATTTCATGCAGATCATCACAATCACTTGCGGACAGCGACG | |
| | | GAGATACTTTTCTTCACTGGTTCCAACAAAGACCCGGACAAAGCCCACGC | |
| | | CTGCTTATTTACGCGGTATCACACAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCATGACCCC | |
| | | GTCACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-010 | VH | GAAGTACAGTTGGTAGAAAGCGGCGGAGGACTTGTGCAGCCAGGTGGATC | 97 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCACG | |
| | | ACGCCCGATAAATGGGGTCAAGGAACAATGGTAACCGTGTCAAGC | |
| | VL | GACGTAGTACTGACCCAAAGCCCCCTTTCTCTCCCAGTAACCCTCGGACA | 98 |
| | | ACCAGCCTCAATTTCATGCAGATCATCACAATCACTTGAGGAGAGCGACG | |
| | | GATACACTTTTCTTGAGTGGTTCCAACAAAGACCCGGACAAAGCCCACGC | |
| | | CTGCTTATTTACGAGGTATCACATAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCCACGACCCC | |
| | | CTGACGTTCGGACAAGGAACTAAGCTCGAAATCAAA | |
| | Heavy | GAAGTACAGTTGGTAGAAAGCGGCGGAGGACTTGTGCAGCCAGGTGGATC | 99 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGCATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCACG | |
| | | ACGCCCGATAAATGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 100 |
| | Chain | ACCAGCCTCAATTTCATGCAGATCATCACAATCACTTGAGGAGAGCGACG | |
| | | GATACACTTTTCTTGAGTGGTTCCAACAAAGACCCGGACAAAGCCCACGC | |
| | | CTGCTTATTTACGAGGTATCACATAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCGACCCACGACCCC | |
| | | CTGACGTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |
| hHA-011 | VH | GAAGTACAGTTGGTAGAAAGCGGCGGAGGACTTGTGCAGCCAGGTGGATC | 101 |
| | | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGAATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCTCG | |
| | | ACCCCCGATTACTGGGGTCAAGGAACAATGGTAACCGTGTCAAGC | |
| | VL | GACGTAGTACTGACCCAAAGCCCCCTTTCTCTCCCAGTAACCCTCGGACA | 102 |
| | | ACCAGCCTCAATTTCATGCAGATCATCACAATCACTTGAGGACAGCGACG | |
| | | GAGGCACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTCAGCTTCGGACAAGGAACTAAGCTCGAAATCAAA | |
| | Heavy | GAAGTACAGTTGGTAGAAAGCGGCGGAGGACTTGTGCAGCCAGGTGGATC | 40 |
| | Chain | TCTCAGACTTTCATGTGCCGCAAGCGGTTTTACTTTTAGTAACTATGGAA | |
| | | TGAACTGGGTTAGACAAGCGCCCGGAAAAGGATTGGAATGGATAGGAATG | |
| | | ATATACTACGACAGCTCCGAGAAACATTATGCCGACTCAGTTAAAGGAAG | |
| | | ATTTACAATATCAAGAGACAATAGCAAAAACACACTTTATCTTCAAATGA | |
| | | ATTCACTGCGAGCCGAGGATACAGCAGTCTATTATTGCGCAAAAGGCTCG | |
| | | ACCCCCGATTACTGGGGTCAAGGAACAATGGTAACCGTGTCAAGCGCGTC | |
| | | GACCAAGGGCCCATCGGTCTTCCCCCTGGCACCCTCCTCCAAGAGCACCT | |
| | | CTGGGGGCACAGCGGCCCTGGGCTGCCTGGTCAAGGACTACTTCCCCGAA | |
| | | CCGGTGACGGTGTCGTGGAACTCAGGCGCCCTGACCAGCGGCGTGCACAC | |
| | | CTTCCCGGCTGTCCTACAGTCCTCAGGACTCTACTCCCTCAGCAGCGTGG | |
| | | TGACCGTGCCCTCCAGCAGCTTGGGCACCCAGACCTACATCTGCAACGTG | |
| | | AATCACAAGCCCAGCAACACCAAGGTGGACAAGAAAGTTGAGCCCAAATC | |
| | | TTGTGACAAAACTCACACATGCCCACCGTGCCCAGCACCTGAAGCCGCGG | |
| | | GGGGACCGTCAGTCTTCCTCTTCCCCCCAAAACCCAAGGACACCCTCATG | |
| | | ATCTCCCGGACCCCTGAGGTCACATGCGTGGTGGTGGACGTGAGCCACGA | |
| | | AGACCCTGAGGTCAAGTTCAACTGGTACGTGGACGGCGTGGAGGTGCATA | |
| | | ATGCCAAGACAAAGCCGCGGGAGGAGCAGTACAACAGCACGTACCGTGTG | |
| | | GTCAGCGTCCTCACCGTCCTGCACCAGGACTGGCTGAATGGCAAGGAGTA | |
| | | CAAGTGCAAGGTCTCCAACAAAGCCCTCCCAGCCCCCATCGAGAAAACCA | |
| | | TCTCCAAAGCCAAAGGGCAGCCCCGAGAACCACAGGTGTACACCCTGCCC | |
| | | CCATCCCGCGAGGAGATGACCAAGAACCAGGTCAGCCTGACCTGCCTGGT | |
| | | CAAAGGCTTCTATCCCAGCGACATCGCCGTGGAGTGGGAGAGCAATGGGC | |
| | | AGCCGGAGAACAACTACAAGACCACGCCTCCCGTGCTGGACTCCGACGGC | |
| | | TCCTTCTTCCTCTACAGCAAGCTCACCGTGGACAAGAGCAGGTGGCAGCA | |
| | | GGGGAACGTCTTCTCATGCTCCGTGATGCATGAGGCTCTGCACAACCACT | |
| | | ACACGCAGAAGAGCCTCTCCCTGTCTCCGGGTAAATGA | |
| | Light | GACGTAGTACTGACCCAAAGCCCCCTTTCTCTCCCAGTAACCCTCGGACA | 64 |
| | Chain | ACCAGCCTCAATTTCATGCAGATCATCACAATCACTTGAGGACAGCGACG | |
| | | GAGGCACTTTTCTTGAGTGGTTCCAACAAAGACCCGGACAAAGCCCACGC | |
| | | CTGCTTATTTACGACGTATCAAGCAGATTCTCAGGAGTTCCAGACAGATT | |
| | | TTCAGGCAGCGGATCCGGCACAGACTTCACCCTTAAAATTAGCAGAGTAG | |
| | | AAGCAGAAGATGTAGGAGTGTATTATTGTTTCCAAGCCACCCACGACCCC | |
| | | CTCAGCTTCGGACAAGGAACTAAGCTCGAAATCAAAAGAACGGTGGCTGC | |
| | | ACCATCTGTCTTCATCTTCCCGCCATCTGATGAGCAGTTGAAATCTGGAA | |
| | | CTGCCTCTGTTGTGTGCCTGCTGAATAACTTCTATCCCAGAGAGGCCAAA | |
| | | GTACAGTGGAAGGTGGATAACGCCCTCCAATCGGGTAACTCCCAGGAGAG | |
| | | TGTCACAGAGCAGGACAGCAAGGACAGCACCTACAGCCTCAGCAGCACCC | |
| | | TGACGCTGAGCAAAGCAGACTACGAGAAACACAAAGTCTACGCCTGCGAA | |
| | | GTCACCCATCAGGGCCTGAGCTCGCCCGTCACAAAGAGCTTCAACAGGGG | |
| | | AGAGTGTTGA | |

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 73.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 75.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 77.

In some embodiments, the anti-HIV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 79.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 71, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 81.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 84, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 85.

In some embodiments, the anti-HIV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 88, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 89.

In some embodiments, the anti-HIV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 92, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 93.

In some embodiments, the anti-HIV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 94, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 93.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 92, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 96.

In some embodiments, the anti-HIV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 99, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 100.

In some embodiments, the anti-HJV described herein is produced by expressing (i) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 40, and/or (ii) a nucleic acid at least 60% (e.g., 60%, 65%, 70%, 75%, 80%, 85%, 90%, 91%, 92%, 93%, 94%, 95%, 96%, 97%, 98%, 99%, or 100%) identical to SEQ ID NO: 64.

In some embodiments, the anti-HJV antibodies described herein can be used for delivering a molecular payload to a target cell or a target tissue (e.g., a cell or tissue that expresses HJV). Accordingly, the anti-HJV antibody described herein can be linked to a molecular payload. The complexes described herein may be used in various applications, e.g., diagnostic or therapeutic applications.

In some embodiments, the complex described herein is used to modulate the activity or function of at least one gene, protein, and/or nucleic acid. In some embodiments, the molecular payload is responsible for the modulation of a gene, protein, and/or nucleic acids. A molecular payload may be a small molecule, protein, nucleic acid, oligonucleotide, or any molecular entity capable of modulating the activity or function of a gene, protein, and/or nucleic acid in a cell. In some embodiments, a molecular payload is an oligonucleotide that targets a disease-associated repeat in muscle cells.

### IV. Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The anti-HJV antibody containing pharmaceutical composition disclosed herein may further comprise a suitable buffer agent. A buffer agent is a weak acid or base used to maintain the pH of a solution near a chosen value after the addition of another acid or base. In some examples, the buffer agent disclosed herein can be a buffer agent capable of maintaining physiological pH despite changes in carbon dioxide concentration (produced by cellular respiration). Exemplary buffer agents include, but are not limited to a HEPES (4-(2-hydroxyethyl)-1-piperazineethanesulfonic acid) buffer, Dulbecco's phosphate-buffered saline (DPBS) buffer, or Phosphate-buffered Saline (PBS) buffer. Such buffers may comprise disodium hydrogen phosphate and sodium chloride, or potassium dihydrogen phosphate and potassium chloride.

In some embodiments, the buffer agent in the pharmaceutical composition described herein may maintain a pH value of about 5-8. For example, the pH of the pharmaceutical composition can be about 7.1, 7.2, 7.3, 7.4, 7.5, 7.6, 7.7, 7.8, 7.9, or 8.0. In other examples, the pharmaceutical composition may have a pH value lower than 7, for example, about 7, 6.8, 6.5, 6.3, 6, 5.8, 5.5, 5.3, or 5.

The pharmaceutical composition described herein comprises one or more suitable salts. A salt is an ionic compound that can be formed by the neutralization reaction of an acid and a base. (Skoog, D. A; West, D. M.; Holler, J. F.; Crouch, S. R. (2004). “chapters 14-16”. Fundamentals of Analytical Chemistry (8th ed.)). Salts are composed of related numbers of cations (positively charged ions) and anions (negative ions) so that the product is electrically neutral (without a net charge).

In some embodiments, the pharmaceutical compositions can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). In some embodiments, the pharmaceutical composition can be formulated for intravenous injection. In some embodiments, the pharmaceutical composition can be formulated for subcutaneous injection.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous or subcutaneous solution bag or vial having a stopper pierceable by a hypodermic injection needle.

As disclosed herein, hemojuvelin antagonists (e.g., anti-HJV antibodies) may be administered by any suitable route. For use in therapy, an effective amount of the antagonist and/or other therapeutic agent can be administered to a subject by any mode that delivers the agent to the desired tissue, e.g., intravenous administration. In some embodiments, hemojuvelin antagonists (e.g., anti-HJV antibodies) are administered subcutaneously and/or intravenously. Other suitable routes of administration include, but are not limited to, oral, parenteral, intraperitoneal, intranasal, sublingual, intratracheal, inhalation, ocular, vaginal, and rectal.

### EXAMPLES

### Example 1: Hemojuvelin Antagonist Promotes Hematological Recovery

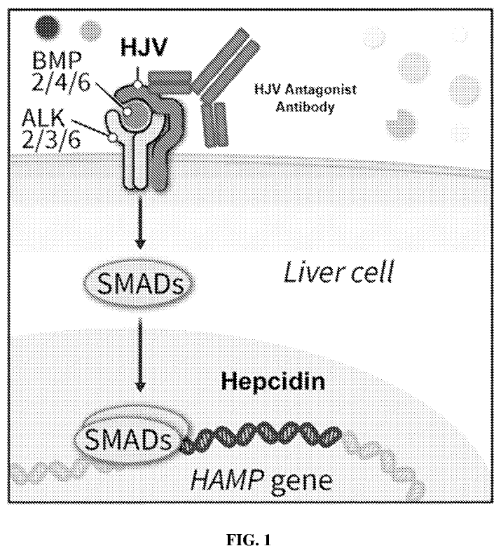

Hemojuvelin (HJV) is a key regulator of hepcidin and iron homeostasis. HJV is a BMP ligand co-receptor that facilitates BMP/SMAD signaling to increase expression of HAMP, the gene that encodes hepcidin (FIG. 1). Loss-of-function mutations in HJV seen in juvenile hemochromatosis are associated with low hepcidin and elevated serum iron levels. These mutations are phenotypically indistinguishable from loss-of-function mutations in HAMP. The present disclosure showed that targeting HJV with an HJV antagonist antibody reduced hepcidin, increased plasma iron, increased RBC count, increased reticulocyte hemoglobin (CHr), increased corpuscular hemoglobin (MCH) and/or increased circulating hemoglobin.

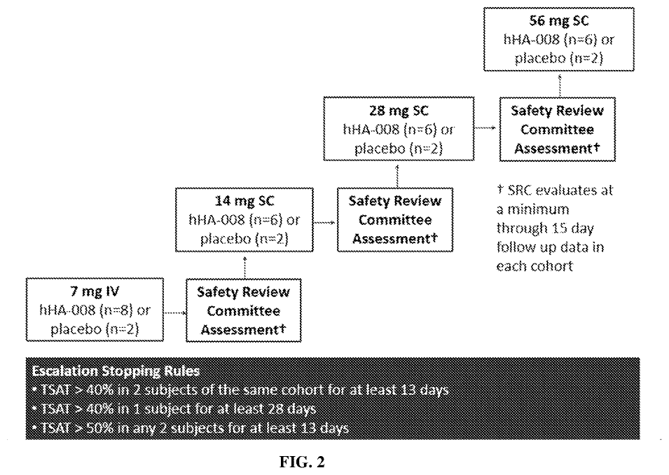

The present disclosure, at least in part, describes a first-in-human, Phase 1a, double-blind, placebo-controlled single-ascending dose study is to evaluate safety, tolerability, pharmacokinetics, and pharmacodynamics of an anti-HJV antibody hHA-008. The design of this study is shown in FIG. 2.

Eligible participants for this study included healthy males and females of non-reproductive potential, ages 18 to 65. Dosing occurred at 7 mg IV, 14 mg SC, 28 mg SC, and 56 mg SC. Safety Review Committee assessments of data through Day 15 were held for each cohort and followed by unblinding. Treatment was allocated 3:1, with 8 participants planned per cohort. Two replacement participants were enrolled in the 7 mg IV cohort and are included in the analyses.

Prior to receiving the hHA-008 antibody, the baseline characteristics were determined. In some embodiments, baseline is described as the last non-missing value before the participant received hHA-008. Data are summarized using “median (range)” at pre-dose, except for “Gender” where “number (N) (percent)” is shown. Abbreviations include transferrin saturation (TSAT %), reticulocyte hemoglobin (CHr), mean corpuscular hemoglobin (MCH), hemoglobin, hematocrit, and red blood cell (RBC) count. Participant number per group is indicated with “n.” Baseline characteristics of the subjects are shown in Table 4.

TABLE 4
Baseline Characteristics

| | Placebo | | 7 mg IV | | 14 mg SC | | 28 mg SC | | 56 mg SC | |
|---|---|---|---|---|---|---|---|---|---|---|
| Characteristic | N = 8 | | n = 8 | | n = 6 | | n = 6 | | N = 6 | |
| Age, years | 57 | (29, 64) | 52 | (41, 62) | 55 | (19, 59) | 56 | (23, 62) | 42 | (29, 60) |
| Gender, | 4 (50)/4 (50) | | 4 (50)/4 (50) | | 3 (50)/3 (50) | | 3 (50)/3 (50) | | 2 (33)/4 (67) | |
| Female/male, N (%) | | | | | | | | | | |
| Hepcidin, ng/mL | 15.0 | (7.0, 20.7) | 10.3 | (2.0, 22.9) | 9.0 | (2.0, 15.0) | 12.5 | (8.6, 16.6) | 8.9 | (5.7, 65.1) |
| TSAT, % | 21 | (14, 27) | 25 | (8, 37) | 25 | (14, 34) | 27 | (18, 71) | 19 | (16, 40) |
| Ferritin, ng/mL | 70 | (39, 118) | 56 | (31, 140) | 51 | (31, 115) | 86 | (49, 156) | 105 | (57, 158) |
| CHr, pg | 34 | (34, 35) | 34 | (27, 35) | 35 | (32, 37) | 35 | (33, 36) | 35 | (32, 37) |
| MCH, pg | 31 | (29, 33) | 30 | (26, 30) | 30 | (29, 34) | 29 | (28, 31) | 31 | (27, 33) |
| Hemoglobin, g/dL | 14.3 | (12.9, 16.2) | 14.0 | (11.7, 15.1) | 14.0 | (12.4, 17.3) | 14.2 | (13.5, 15.6) | 14.4 | (12.6, 16.1) |
| Hematocrit, % | 43 | (39, 48) | 42 | (37, 47) | 41 | (37, 48) | 42 | (38, 45) | 43 | (38, 46) |
| RBC, 10¹²/L | 4.6 | (4.4, 5.5) | 4.7 | (4.2, 5.2) | 4.6 | (4.3, 5.2) | 5.0 | (4.3, 5.1) | 4.8 | (4.1, 5.4) |

After receiving hHA-008 at the prescribed dose, primary endpoints for the safety and tolerability objectives included: adverse events, clinical laboratory assessments, vital signs, physical examinations, and electrocardiograms (FIG. 2). No serious adverse events (AEs), ≥Grade 2 AEs, or AEs leading to study withdrawal were reported.

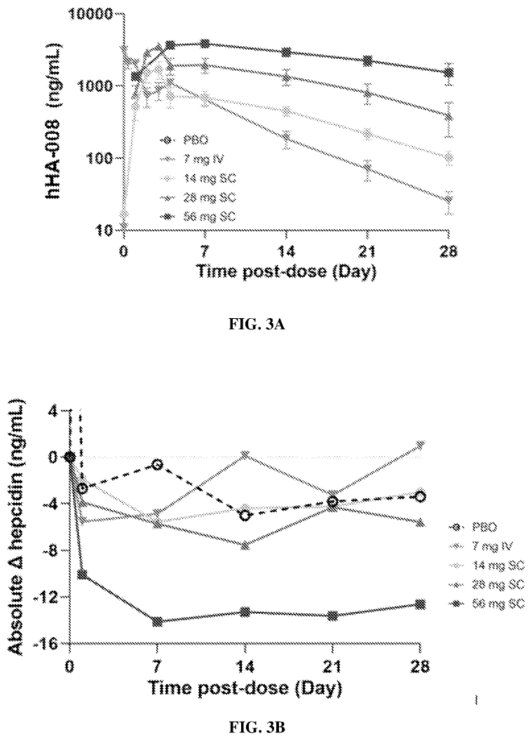

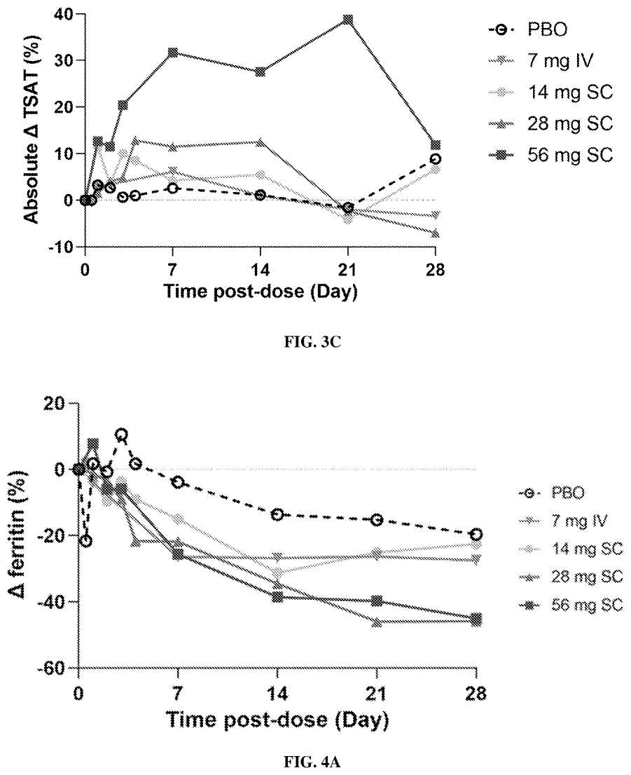

Secondary endpoints included standard pharmacokinetic parameters and the following pharmacodynamic parameters: hepcidin, transferrin saturation (TSAT %), and exploratory hematology biomarkers. Serum samples were analyzed for hHA-008 antibody concentrations using a validated electrochemiluminescence immunoassay method. Data were summarized using descriptive statistics and showed increasing serum concentrations of hHa-008 in a dose-dependent manner (FIG. 3A). The results showed that increasing doses of hHA-008 antibody decreased hepcidin (FIG. 3B) and increased TSAT % (FIG. 3C) in the treatment group. FIG. 3A shows hHA-008 antibody concentration-time profiles plotted as mean with error bars representing standard error of the mean in 7 mg IV, 14 mg SC, 28 mg SC and 56 mg SC. FIGS. 3B-3C shows mean absolute change (A) from baseline in hepcidin (FIG. 3B) and transferrin saturation (TSAT %) (FIG. 3C). Data are shown for pooled placebo (PBO) (n=8), 7 mg IV (n=8), 14 mg SC (n=6), 28 mg SC (n=6), and 56 mg SC (n=6).

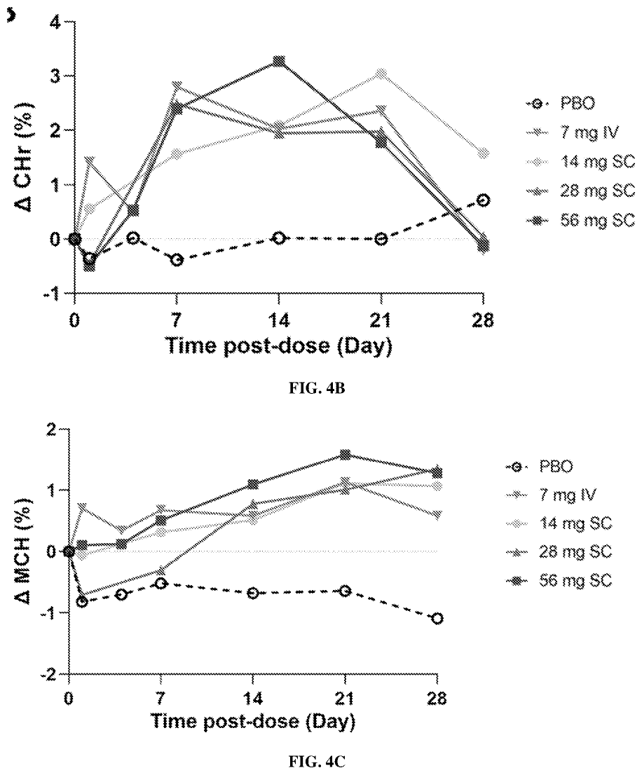

Additional hematology biomarkers were evaluated to assess the effect of hHA-008 antibody on iron storage, hemoglobin production, and erythropoiesis. The results showed that hHA-008 antibody treatment reduced ferritin levels (FIG. 4A), increased reticulocyte hemoglobin (CHr) (FIG. 4B) and increased mean corpuscular hemoglobin (MCH) (FIG. 4C) in the treatment groups compared to the placebo group, suggesting that hHA-008 antibody induced iron mobilization from ferritin iron stores to promote hemoglobin production.

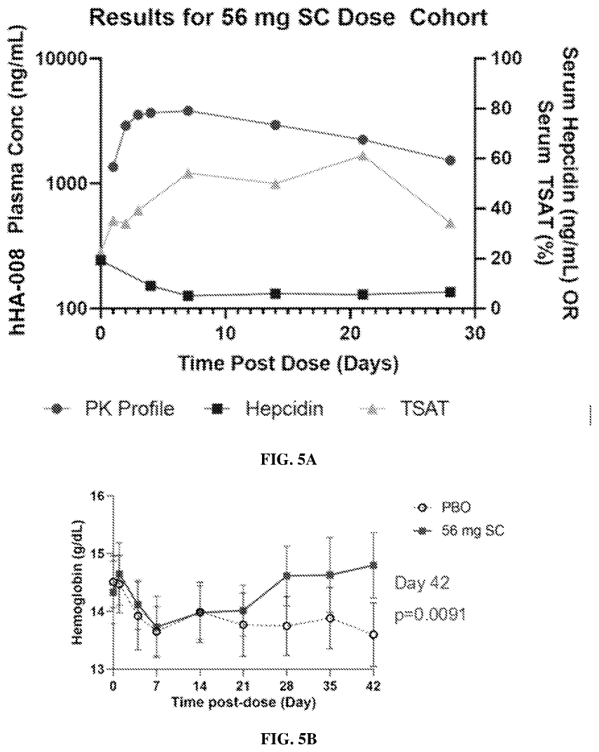

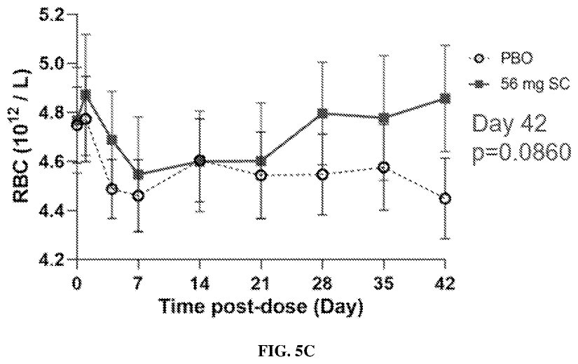

Further, a single dose of 56 mg SC hHA-008 led to robust hepcidin and TSAT responses that increased erythropoiesis and hemoglobin in healthy volunteers. FIG. 5A shows mean PK profile (left Y-axis) and PD response (right Y-axis) measured using hepcidin and transferrin saturation (TSAT) for the 56 mg SC cohort (n=6). Blood was drawn from subjects twice in the first week after hHA-008 treatment, which led to decreased hemoglobin and RBC count from baseline. However, both hemoglobin level and RBC count recovered to baseline level faster in the group treated with 56 mg of hHA-008 compared to the group received the placebo, indicating the ability of hHa-008 treatment to help promote erythropoiesis. FIGS. 5B-5C show mean with error bars representing standard error of the mean for hemoglobin (FIG. 5B) and red blood cell (RBC) count (FIG. 5C) for pooled placebo (PBO) (n=8) and 56 mg SC (n=6). Asterisk (*) indicates statistically significant, two-sample t-test comparison of Day 42 hemoglobin, assessed as change from baseline. This study provides clinical proof of a mechanism that inhibiting HJV with an HJV antagonist antibody reduced hepcidin and increased circulating iron availability.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the application describes “a composition comprising A and B,” the application also contemplates the alternative embodiments “a composition consisting of A and B” and “a composition consisting essentially of A and B.”

Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

What is claimed is:

1. A method for treating a subject having a blood loss condition, the method comprising administering to the subject a hemojuvelin antagonist.

2. The method of claim 1, wherein the hemojuvelin antagonist is administered in an amount effective for promoting hematological recovery.

3. The method of claim 2, wherein the hematological recovery comprises recovery of erythropoiesis, reticulocyte hemoglobin content (CHr), mean corpuscular hemoglobin (MCH) and/or circulating hemoglobin levels to baseline levels prior to the blood loss condition.

4. The method of claim 2 or 3, wherein the hematological recovery is achieved within a shorter duration than would be achieved by a control subject who did not receive the hemojuvelin antagonist.

5. The method of any one of claims 2-4, wherein the hematological recovery comprises recovery of erythropoiesis, reticulocyte hemoglobin (CHr), mean corpuscular hemoglobin (MCH) and/or circulating hemoglobin levels to baseline levels within 3 days, 5 days, 1 week, two weeks, three weeks, or four weeks.

6. The method of any one of claims 1-5, wherein the blood loss condition comprises chronic blood loss.

7. The method of any one of claims 1-5, wherein the blood loss condition comprises acute blood loss.

8. The method of any one of claims 1-7, wherein the blood loss condition comprises iatrogenic blood loss.

9. The method of any one of claims 1-8, wherein the blood loss condition comprises a phlebotomy procedure.

10. The method of any one of claims 1-8, wherein the blood loss condition comprises a surgical procedure.

11. The method of any one of claims 1-9, wherein the blood loss condition comprises a blood donation procedure.

12. The method of any one of claims 1-10, wherein the blood loss condition comprises a bleeding wound.

13. The method of claim 12, wherein the bleeding wound is an internal bleeding wound.

14. The method of claim 13, wherein the internal bleeding wound is selected from ruptured blood vessels, organ contusion, organ rupture, and hematoma.

15. The method of claim 12, wherein the bleeding wound is an external bleeding wound.

16. The method of claim 15, wherein the external bleeding wound is selected from a cut, a stab, a puncture, an avulsion, an incision, and a penetration.

17. The method of any one of claims 1-7, wherein the blood loss condition comprises a disease.

18. The method of claim 17, wherein the disease is a gastrointestinal (GI) disease.

19. The method of claim 18, wherein the GI disease comprises blood loss due to esophageal varices, gastritis, gastric ulcer, duodenal ulcer, diverticulosis, Meckel's diverticulum, intestinal polyps, inflammatory bowel disease (IBD), hemorrhoids, celiac disease or colorectal cancer.

20. The method of claim 17, wherein the disease a genitourinary disease.

21. The method of claim 20, wherein the genitourinary disease comprises blood loss due to menorrhagia, fibroid, endometriosis, bladder tumors, urinary tract infection (UTI), or renal stones.

22. The method of claim 17, wherein the disease is an infectious disease.

23. The method of claim 22, wherein infectious disease comprises blood loss due to a viral hemorrhagic fever selected from Dengue fever, Ebola virus disease, Lassa fever, Hantavirus pulmonary syndrome, Marburg virus disease, and yellow fever.

24. The method of claim 22, wherein the infectious disease comprises blood loss due to a bacterial infectious disease selected from sepsis, bacterial vaginosis, Lemierre's syndrome, and tuberculosis.

25. The method of claim 22, wherein the infectious disease comprises blood loss due to a parasitic infectious disease selected from malaria, Trichuriasis, and Schistosomiasis.

26. The method of any one of any one of claims 1-25, wherein prior to administration, the subject has a hemoglobin level of at least 6 g/dl.

27. The method of any one of claims 1-26, wherein prior to administration, the subject has a hemoglobin level in the range of 7-12 g/dl.

28. The method of any one of claims 1-27, wherein prior to administration, the subject has a hemoglobin level in the range of 7-11 g/dl.

29. The method of any one of claims 1-28, wherein prior to administration, the subject has a hemoglobin level in the range of 7-10 g/dl.

30. The method of claims 26-29, wherein after administration, the subject's hemoglobin level increases by 1-3 g/dl.

31. The method of claim 30, wherein after administration, the subject's hemoglobin level increases by 1-3 g/dl within 1 week, two weeks, three weeks, four weeks, five weeks, or six weeks.

32. The method of any one of claims 1-31, wherein prior to administration, the subject has a ferritin level of up to 5000 ng/ml.

33. The method of any one of claims 1-32, wherein prior to administration, the subject has a ferritin level in the range of 14-150 ng/ml

34. The method of claim 33, wherein after administration, the subject's ferritin level decreases by 15%-50% compared to the ferritin level prior to administration.

35. The method of any one of claims 1-34, wherein prior to administration, the subject has a transferrin saturation (TSAT %) level in the range of 15%-30%.

36. The method of claim 35, wherein after administration, the subject has a TSAT % level in the range of 30%-50%.

37. The method of claim 36, wherein the subject has a TSAT % level in the range of 30%-50% within 1 week, two weeks, three weeks, four weeks, five weeks, or six weeks.

38. The method of any one of claims 1-37, wherein prior to administration, the subject has a reticulocyte hemoglobin (CHr) level in the range of 20-40 pg.

39. The method of claim 38, wherein after administration, the subject's CHr level increases by 1%-5% compared to the CHr level prior to administration.

40. The method of claim 39, wherein the subject's CHr level increases by 1%-5% within 1 day, 2 days, 3 days, 4 days, 5 days, 6 days, one week, or two weeks.

41. The method of any one of claims 1-40, wherein prior to administration, the subject has a hepcidin level in the range of 5-75 ng/ml.

42. The method of claim 41, wherein after administration, the subject's hepcidin level decreases by 2-60 ng/ml compared to the hepcidin level prior to administration.

43. The method of any one of claims 1-42, wherein prior to administration, the subject has a red blood cell count in the range of 3×10¹² to 6×10¹² cells/L.

44. The method of claim 43, wherein after administration, the subject's red blood cell count increases by at least 0.5×10¹² cells/L compared to the red blood cell count prior to administration.

45. The method of any one of claims 1-44, wherein prior to administration, the subject has a mean corpuscular hemoglobin (MCH) level of 15-50 pg.

46. The method of claim 45, wherein after administration, the subject's MCH level increases by 1%-5% compared to the MCH level prior to administration.

47. The method of any one of claims 1-46, wherein the subject does not have a functional iron deficiency prior to administration.

48. The method of any one of claims 1-47, wherein the subject does not have anemia associated with inflammation prior to administration.

49. The method of any one of claims 1-47, wherein the subject has anemia associated with inflammation prior to administration.

50. The method of any one of claims 1-49, wherein the blood loss condition comprises persistent blood loss.

51. The method of any one of claims 1-49, wherein the blood loss condition comprises more than one intermittent blood loss instance up to one week apart, up to two weeks apart, or up to one month apart.

52. The method of any one of claims 1-51, wherein the blood loss condition comprises loss of up to 10% of the subject's total blood volume.

53. The method of any one of claims 1-52, wherein the blood loss is in the range of 1-10% of the subject's total blood volume.

54. The method of any one of claims 1-53, wherein the blood loss condition comprises persistent blood loss which persists at least 1 hour, at least 4 hours, at least 8 hours, at least 12 hours, at least 16 hours, at least 20 hours, at least 1 day, at least 3 days, at least 5 days, at least 8 days, at least 10 days, at least two weeks, at least three weeks, at least one month, at least two months, or at least three months.

55. The method of any one of claims 1-53, wherein the blood loss condition comprises intermittent blood loss instance wherein each instance of blood loss comprises loss of up to 10% of the subject's total blood volume.

56. The method of claim 55, wherein each instance of blood loss occurs up to 1 hour, up to 4 hours, up to 8 hours, up to 12 hours, up to 16 hours, up to 20 hours, up to 1 day, up to 3 days, up to 5 days, up to 8 days, up to 10 days, or up to two weeks apart.

57. The method of any one of claims 1-56, wherein the subject has a ferritin level of 14-80 ng/ml due to the blood loss.

58. The method of claim 57, wherein the subject has a serum iron of at least 40 μg/dL.

59. The method of any one of claims 1-58, wherein the hemojuvelin antagonist is an anti-hemojuvelin antibody.

60. The method of any one of claims 1-59, wherein the hemojuvelin antagonist is administered to the subject weekly, twice a month, or once a month.

61. The method of any one of claims 1-60, wherein the subject is not identified as requiring blood transfusion.

62. The method of any one of claims 1-61, wherein the subject is identified as requiring blood transfusion prior to administration of the hemojuvelin antagonist.

63. The method of any one of claims 1-62, wherein the hemojuvelin antagonist is administered in combination with oral iron supplement or iron injection.

64. The method of any one of claims 1-63, wherein the hemojuvelin antagonist is administered while the subject is on a gluten-free diet.

65. The method of any one of claims 1-64, wherein the hemojuvelin antagonist is administered in combination with a therapeutic agent.

66. The method of claim 65, wherein the therapeutic agent is Erythropoietin (EPO).

67. The method of claim 65, wherein the therapeutic agent is Luspatercept.

68. The method of claim 65, wherein the therapeutic agent is Hypoxia-inducible factor prolyl hydroxylase inhibitor (HIF-PHI).

69. The method of claim 65, wherein the therapeutic agent is for treating the disease that is associated with blood loss.

70. The method of any one of claims 1-69, wherein the hemojuvelin antagonist is administered subcutaneously.

71. The method of any one of claims 1-70, wherein the hemojuvelin antagonist is administered intravenously.

72. The method of claim 71, wherein the subject is administered the hemojuvelin antagonist on multiple occasions.

73. The method of claim 72, wherein the occasions are on a monthly interval.


SEQUENCE LISTING (129 sequences)

SEQ ID NO: 1 - protein, 5 aa, synthetic construct
  REGION 1..5: note = Synthethic Polypeptide
NYGMN

SEQ ID NO: 2 - protein, 17 aa, synthetic construct
  REGION 1..17: note = Synthethic Polypeptide
MIYYDSSEKHYADSVKG

SEQ ID NO: 3 - protein, 6 aa, synthetic construct
  REGION 1..6: note = Synthethic Polypeptide
GTTPDY

SEQ ID NO: 4 - protein, 16 aa, synthetic construct
  REGION 1..16: note = Synthethic Polypeptide
RSSQSLETSDGDTFLE

SEQ ID NO: 5 - protein, 7 aa, synthetic construct
  REGION 1..7: note = Synthethic Polypeptide
EVSTRFS

SEQ ID NO: 6 - protein, 9 aa, synthetic construct
  REGION 1..9: note = Synthethic Polypeptide
FQVTHDPMT

SEQ ID NO: 7 - protein, 115 aa, synthetic construct
  REGION 1..115: note = Synthethic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSS

SEQ ID NO: 8 - protein, 112 aa, synthetic construct
  REGION 1..112: note = Synthethic Polypeptide
DVVLTQSPLSLPVTLGQPASISCRSSQSLETSDGDTFLEWFQQRPGQSPRLLIYEVSTRFSGVPDRFSGSGSGTDFTLKISRVEAEDVGVYYCFQVTHDPMTFGQGTKLEIK

SEQ ID NO: 9 - protein, 5 aa, synthetic construct
  REGION 1..5: note = Synthethic Polypeptide
YYGMN

SEQ ID NO: 10 - protein, 17 aa, synthetic construct
  REGION 1..17: note = Synthethic Polypeptide
MIYYDSSDKHYADSVKG

SEQ ID NO: 11 - protein, 6 aa, synthetic construct
  REGION 1..6: note = Synthethic Polypeptide
GTTPDV

SEQ ID NO: 12 - protein, 6 aa, synthetic construct
  REGION 1..6: note = Synthethic Polypeptide
GTTPDK

SEQ ID NO: 13 - protein, 6 aa, synthetic construct
  REGION 1..6: note = Synthethic Polypeptide
GSTPDY

SEQ ID NO: 14 - protein, 16 aa, synthetic construct
  REGION 1..16: note = Synthethic Polypeptide
RSSQSLESSDGDTFLE

SEQ ID NO: 15 - protein, 16 aa, synthetic construct
  REGION 1..16: note = Synthethic Polypeptide
RSSQSLEESDGYTFLE

SEQ ID NO: 16 - protein, 16 aa, synthetic construct
  REGION 1..16: note = Synthethic Polypeptide
RSSQSLEDSDGGTFLE

SEQ ID NO: 17 - protein, 16 aa, synthetic construct
  REGION 1..16: note = Synthethic Polypeptide
RSSQSLEESDGYTFLH

SEQ ID NO: 18 - protein, 7 aa, synthetic construct
  REGION 1..7: note = Synthethic Polypeptide
EVSNRFS

SEQ ID NO: 19 - protein, 7 aa, synthetic construct
  REGION 1..7: note = Synthethic Polypeptide
DVSTRFS

SEQ ID NO: 20 - protein, 7 aa, synthetic construct
  REGION 1..7: note = Synthethic Polypeptide
DVSERFS

SEQ ID NO: 21 - protein, 7 aa, synthetic construct
  REGION 1..7: note = Synthethic Polypeptide
DVSSRFS

SEQ ID NO: 22 - protein, 7 aa, synthetic construct
  REGION 1..7: note = Synthethic Polypeptide
AVSHRFS

SEQ ID NO: 23 - protein, 7 aa, synthetic construct
  REGION 1..7: note = Synthethic Polypeptide
EVSHRFS

SEQ ID NO: 24 - protein, 9 aa, synthetic construct
  REGION 1..9: note = Synthethic Polypeptide
MQVTHDPLT

SEQ ID NO: 25 - protein, 9 aa, synthetic construct
  REGION 1..9: note = Synthethic Polypeptide
FQVTHDPVT

SEQ ID NO: 26 - protein, 9 aa, synthetic construct
  REGION 1..9: note = Synthethic Polypeptide
FQATYDPLT

SEQ ID NO: 27 - protein, 9 aa, synthetic construct
  REGION 1..9: note = Synthethic Polypeptide
FQATHDPLT

SEQ ID NO: 28 - protein, 9 aa, synthetic construct
  REGION 1..9: note = Synthethic Polypeptide
FQATHDPVT

SEQ ID NO: 29 - protein, 9 aa, synthetic construct
  REGION 1..9: note = Synthethic Polypeptide
FQATHDPLS

SEQ ID NO: 30 - protein, 112 aa, synthetic construct
  REGION 1..112: note = Synthethic Polypeptide
DVVLTQSPLSLPVTLGQPASISCRSSQSLETSDGDTFLEWFQQRPGQSPRLLIYEVSSRFSGVPDRFSGSGSGTDFTLKISRVEAEDVGVYYCMQVTHDPLTFGQGTKLEIK

SEQ ID NO: 31 - protein, 112 aa, synthetic construct
  REGION 1..112: note = Synthethic Polypeptide
DVVLTQSPLSLPVTLGQPASISCRSSQSLETSDGDTFLEWFQQRPGQSPRLLIYEVSNRFSGVPDRFSGSGSGTDFTLKISRVEAEDVGVYYCFQVTHDPVTFGQGTKLEIK

SEQ ID NO: 32 - protein, 112 aa, synthetic construct
  REGION 1..112: note = Synthethic Polypeptide
DVVLTQSPLSLPVTLGQPASISCRSSQSLESSDGDTFLEWFQQRPGQSPRLLIYDVSTRFSGVPDRFSGSGSGTDFTLKISRVEAEDVGVYYCFQVTHDPVTFGQGTKLEIK

SEQ ID NO: 33 - protein, 112 aa, synthetic construct
  REGION 1..112: note = Synthethic Polypeptide
DVVLTQSPLSLPVTLGQPASISCRSSQSLEESDGYTFLEWFQQRPGQSPRLLIYDVSERFSGVPDRFSGSGSGTDFTLKISRVEAEDVGVYYCFQATYDPLTFGQGTKLEIK

SEQ ID NO: 34 - protein, 115 aa, synthetic construct
  REGION 1..115: note = Synthethic Polypeptide
EVQLVESGGGLVQPGGSLRLSCAASGFTFSYYGMNWIRQAPGKGLEWIGMIYYDSSEKHYADSVKGRFTISRDNSKNTLYLQMNSLRAEDTAVYYCAKGTTPDYWGQGTMVTVSS

SEQ ID NO: 35 - protein, 112 aa, synthetic construct
  REGION 1..112: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLTFGQGTKLEIK

SEQ ID NO: 36 - protein, 115 aa, synthetic construct
  REGION 1..115: note = Synthethic Polypeptide
EVQLVESGGGLVQPGGSLRLSCAASGFTFSNYGMNWIRQAPGKGLEWIGMIYYDSSDKHYADSVKGRFTISRDNSKNTLYLQMNSLRAEDTAVYYCAKGTTPDVWGQGTMVTVSS

SEQ ID NO: 37 - protein, 112 aa, synthetic construct
  REGION 1..112: note = Synthethic Polypeptide
DVVLTQSPLSLPVTLGQPASISCRSSQSLEESDGYTFLHWFQQRPGQSPRLLIYEVSNRFSGVPDRFSGSGSGTDFTLKISRVEAEDVGVYYCFQATHDPVTFGQGTKLEIK

SEQ ID NO: 38 - protein, 115 aa, synthetic construct
  REGION 1..115: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSS

SEQ ID NO: 39 - protein, 112 aa, synthetic construct
  REGION 1..112: note = Synthethic Polypeptide
DVVLTQSPLSLPVTLGQPASISCRSSQSLEESDGYTFLHWFQQRPGQSPRLLIYEVSTRFSGVPDRFSGSGSGTDFTLKISRVEAEDVGVYYCFQATHDPLTFGQGTKLEIK

SEQ ID NO: 40 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggaatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggctcgacccccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - protein, 112 aa, synthetic construct
  REGION 1..112: note = Synthethic Polypeptide
DVVLTQSPLSLPVTLGQPASISCRSSQSLADSDGDTFLHWFQQRPGQSPRLLIYAVSHRFSGVPDRFSGSGSGTDFTLKISRVEAEDVGVYYCFQATHDPVTFGQGTKLEIK

SEQ ID NO: 42 - protein, 115 aa, synthetic construct
  REGION 1..115: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDKWGQGTMVTVSS

SEQ ID NO: 43 - protein, 112 aa, synthetic construct
  REGION 1..112: note = Synthethic Polypeptide
DVVLTQSPLSLPVTLGQPASISCRSSQSLEESDGYTFLEWFQQRPGQSPRLLIYEVSHRFSGVPDRFSGSGSGTDFTLKISRVEAEDVGVYYCFQATHDPLTFGQGTKLEIK

SEQ ID NO: 44 - protein, 115 aa, synthetic construct
  REGION 1..115: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGSTPDYWGQGTMVTVSS

SEQ ID NO: 45 - protein, 112 aa, synthetic construct
  REGION 1..112: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLSFGQGTKLEIK

SEQ ID NO: 46 - protein, 330 aa, synthetic construct
  REGION 1..330: note = Synthethic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 107 aa, Homo sapiens [sequence omitted]

SEQ ID NO: 48 - protein, 330 aa, synthetic construct
  REGION 1..330: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 49 - protein, 7 aa, synthetic construct
  REGION 1..7: note = Synthethic Polypeptide
EVSSRFS

SEQ ID NO: 50 - protein, 16 aa, synthetic construct
  REGION 1..16: note = Synthethic Polypeptide
RSSQSLADSDGDTFLH

SEQ ID NO: 51 - protein, 445 aa, synthetic construct
  REGION 1..445: note = Synthethic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 219 aa, synthetic construct
  REGION 1..219: note = Synthethic Polypeptide
DVVLTQSPLSLPVTLGQPASISCRSSQSLETSDGDTFLEWFQQRPGQSPRLLIYEVSTRFSGVPDRFSGSGSGTDFTLKISRVEAEDVGVYYCFQVTHDPMTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 219 aa, synthetic construct
  REGION 1..219: note = Synthethic Polypeptide
DVVLTQSPLSLPVTLGQPASISCRSSQSLETSDGDTFLEWFQQRPGQSPRLLIYEVSSRFSGVPDRFSGSGSGTDFTLKISRVEAEDVGVYYCMQVTHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 219 aa, synthetic construct
  REGION 1..219: note = Synthethic Polypeptide
DVVLTQSPLSLPVTLGQPASISCRSSQSLETSDGDTFLEWFQQRPGQSPRLLIYEVSNRFSGVPDRFSGSGSGTDFTLKISRVEAEDVGVYYCFQVTHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 219 aa, synthetic construct
  REGION 1..219: note = Synthethic Polypeptide
DVVLTQSPLSLPVTLGQPASISCRSSQSLESSDGDTFLEWFQQRPGQSPRLLIYDVSTRFSGVPDRFSGSGSGTDFTLKISRVEAEDVGVYYCFQVTHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 219 aa, synthetic construct
  REGION 1..219: note = Synthethic Polypeptide
DVVLTQSPLSLPVTLGQPASISCRSSQSLEESDGYTFLEWFQQRPGQSPRLLIYDVSERFSGVPDRFSGSGSGTDFTLKISRVEAEDVGVYYCFQATYDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 445 aa, synthetic construct
  REGION 1..445: note = Synthethic Polypeptide
EVQLVESGGGLVQPGGSLRLSCAASGFTFSYYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 219 aa, synthetic construct
  REGION 1..219: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 445 aa, synthetic construct
  REGION 1..445: note = Synthethic Polypeptide
EVQLVESGGGLVQPGGSLRLSCAASGFTFSNYGMNWIRQAPGKGLEWIGMIYYDSSDKHYADSVKGRFTISRDNSKNTLYLQMNSLRAEDTAVYYCAKGTTPDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, synthetic construct
  REGION 1..219: note = Synthethic Polypeptide
DVVLTQSPLSLPVTLGQPASISCRSSQSLEESDGYTFLHWFQQRPGQSPRLLIYEVSNRFSGVPDRFSGSGSGTDFTLKISRVEAEDVGVYYCFQATHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 219 aa, synthetic construct
  REGION 1..219: note = Synthethic Polypeptide
DVVLTQSPLSLPVTLGQPASISCRSSQSLEESDGYTFLHWFQQRPGQSPRLLIYEVSTRFSGVPDRFSGSGSGTDFTLKISRVEAEDVGVYYCFQATHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 63 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPGK

SEQ ID NO: 64 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggacagcgacggaggcacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgtttccaagccacccacgaccccctcagcttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 65 - protein, 219 aa, synthetic construct
  REGION 1..219: note = Synthethic Polypeptide
DVVLTQSPLSLPVTLGQPASISCRSSQSLADSDGDTFLHWFQQRPGQSPRLLIYAVSHRFSGVPDRFSGSGSGTDFTLKISRVEAEDVGVYYCFQATHDPVTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDKWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 219 aa, synthetic construct
  REGION 1..219: note = Synthethic Polypeptide
DVVLTQSPLSLPVTLGQPASISCRSSQSLEESDGYTFLEWFQQRPGQSPRLLIYEVSHRFSGVPDRFSGSGSGTDFTLKISRVEAEDVGVYYCFQATHDPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 445 aa, synthetic construct
  REGION 1..445: note = Synthethic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGSTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 219 aa, synthetic construct
  REGION 1..219: note = Synthethic Polypeptide
DVVLTQSPLSLPVTLGQPASISCRSSQSLEDSDGGTFLEWFQQRPGQSPRLLIYDVSSRFSGVPDRFSGSGSGTDFTLKISRVEAEDVGVYYCFQATHDPLSFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaattggattagacaagcgcccggaaaaggattggaatggataggaatgatatactacgatagctcagaaaaacattatgccgactcagttaaaggaagatttacaatatcaagagacaatagcaaaaacacactttatcttcaaatgaattcactgcgagccgaggatacagcagtctattattgcgcaaaaggaacaacaccagattattggggtcaaggaacaatggtaaccgtgtcaagc

SEQ ID NO: 71 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaattggattagacaagcgcccggaaaaggattggaatggataggaatgatatactacgatagctcagaaaaacattatgccgactcagttaaaggaagatttacaatatcaagagacaatagcaaaaacacactttatcttcaaatgaattcactgcgagccgaggatacagcagtctattattgcgcaaaaggaacaacaccagattattggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 72 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaacgagattctcaggagttccagacagattttcaggcagcggatccggcacagacttcacccttaaaattagcagagtagaagcagaagatgtaggagtgtattattgttttcaagtcacccacgaccccatgacgttcggacaaggaactaagctcgaaatcaaa

SEQ ID NO: 73 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaacgagattctcaggagttccagacagattttcaggcagcggatccggcacagacttcacccttaaaattagcagagtagaagcagaagatgtaggagtgtattattgttttcaagtcacccacgaccccatgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 74 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaagcagattctcaggagttccagacagattttcaggcagcggatccggcacagacttcacccttaaaattagcagagtagaagcagaagatgtaggagtgtattattgtatgcaagtcacccacgaccccctgaccttcggacaaggaactaagctcgaaatcaaa

SEQ ID NO: 75 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagacgagcgacggagacacttttcttgagtggttccaacaaagacccggacaaagcccacgcctgcttatttacgaggtatcaagcagattctcaggagttccagacagattttcaggcagcggatccggcacagacttcacccttaaaattagcagagtagaagcagaagatgtaggagtgtattattgtatgcaagtcacccacgaccccctgaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 76 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagaccagcgacggagatacttttcttgagtggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagtcacccacgaccccgtcacgttcggacaaggaactaagctcgaaatcaaa

SEQ ID NO: 77 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagaccagcgacggagatacttttcttgagtggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagtcacccacgaccccgtcacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 78 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgagtccagcgacggagacacttttcttgagtggttccaacaaagacccggacaaagcccacgcctgcttatttacgatgtatcaactagattctcaggagttccagacagattttcaggcagcggatccggcacagacttcacccttaaaattagcagagtagaagcagaagatgtaggagtgtattattgttttcaagtcacccatgaccccgtgaccttcggacaaggaactaagctcgaaatcaaa

SEQ ID NO: 79 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgagtccagcgacggagacacttttcttgagtggttccaacaaagacccggacaaagcccacgcctgcttatttacgatgtatcaactagattctcaggagttccagacagattttcaggcagcggatccggcacagacttcacccttaaaattagcagagtagaagcagaagatgtaggagtgtattattgttttcaagtcacccatgaccccgtgaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 80 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggaaagcgacggatacacttttcttgaatggttccaacaaagacccggacaaagcccacgcctgcttatttacgatgtatcagaaagattctcaggagttccagacagattttcaggcagcggatccggcacagacttcacccttaaaattagcagagtagaagcagaagatgtaggagtgtattattgtttccaagcgacctacgaccccctcaccttcggacaaggaactaagctcgaaatcaaa

SEQ ID NO: 81 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggaaagcgacggatacacttttcttgaatggttccaacaaagacccggacaaagcccacgcctgcttatttacgatgtatcagaaagattctcaggagttccagacagattttcaggcagcggatccggcacagacttcacccttaaaattagcagagtagaagcagaagatgtaggagtgtattattgtttccaagcgacctacgaccccctcaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 82 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagttactatggaatgaactggattagacaagcgcccggaaaaggattggaatggataggcatgatatactacgacagctcggagaaacattatgccgactcagttaaaggaagatttacaatatcaagagacaatagcaaaaacacactttatcttcaaatgaattcactgcgagccgaggatacagcagtctattattgcgcaaaagggacgacgcccgattactggggtcaaggaacaatggtaaccgtgtcaagc

SEQ ID NO: 83 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggacagcgacggaggaacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgttttcaagcgacccacgaccccctgacgttcggacaaggaactaagctcgaaatcaaa

SEQ ID NO: 84 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagttactatggaatgaactggattagacaagcgcccggaaaaggattggaatggataggcatgatatactacgacagctcggagaaacattatgccgactcagttaaaggaagatttacaatatcaagagacaatagcaaaaacacactttatcttcaaatgaattcactgcgagccgaggatacagcagtctattattgcgcaaaagggacgacgcccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 85 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggacagcgacggaggaacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgttttcaagcgacccacgaccccctgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 86 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaactggataagacaagcgcccggaaaaggattggaatggataggcatgatatactacgacagctcggacaaacattatgccgactcagttaaaggaagatttacaatatcaagagacaatagcaaaaacacactttatcttcaaatgaattcactgcgagccgaggatacagcagtctattattgcgcaaaagggacgacgccggatgtatggggtcaaggaacaatggtaaccgtgtcaagc

SEQ ID NO: 87 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaagagagcgacggatacacttttcttcattggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagccacccacgaccccgtgacgttcggacaaggaactaagctcgaaatcaaa

SEQ ID NO: 88 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaactggataagacaagcgcccggaaaaggattggaatggataggcatgatatactacgacagctcggacaaacattatgccgactcagttaaaggaagatttacaatatcaagagacaatagcaaaaacacactttatcttcaaatgaattcactgcgagccgaggatacagcagtctattattgcgcaaaagggacgacgccggatgtatggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 89 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaagagagcgacggatacacttttcttcattggttccaacaaagacccggacaaagcccacgcctgcttatttacgaggtatcaaacagattctcaggagttccagacagattttcaggcagcggatccggcacagacttcacccttaaaattagcagagtagaagcagaagatgtaggagtgtattattgtttccaagccacccacgaccccgtgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 90 - DNA, 345 bp, synthetic construct
  misc_feature 1..345: note = Synthethic Polynucleotide
  source 1..345: mol_type = other DNA
gaagtacagttggtagaaagcggcggaggacttgtgcaaccaggtggatctctcagactttcatgtgccgcaagcggttttacttttagtaactatggaatgaactgggttagacaagcgcccggaaaaggattggaatggataggcatgatatattacgacagctcggagaaacattatgccgactcagttaaaggaagatttacaatatcaagagacaatagcaaaaacacactttatcttcaaatgaattcactgcgagccgaggatacagcagtctattattgcgcaaaagggaccacccccgattactggggtcaaggaacaatggtaaccgtgtcaagc

SEQ ID NO: 91 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaagaaagcgacggatacacttttcttcactggttccaacaaagacccggacaaagcccacgcctgcttatttacgaggtatcaaccagattctcaggagttccagacagattttcaggcagcggatccggcacagacttcacccttaaaattagcagagtagaagcagaagatgtaggagtgtattattgtttccaagccacccacgaccccctgaccttcggacaaggaactaagctcgaaatcaaa

SEQ ID NO: 92 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcaaccaggtggatctctcagactttcatgtgccgcaagcggttttacttttagtaactatggaatgaactgggttagacaagcgcccggaaaaggattggaatggataggcatgatatattacgacagctcggagaaacattatgccgactcagttaaaggaagatttacaatatcaagagacaatagcaaaaacacactttatcttcaaatgaattcactgcgagccgaggatacagcagtctattattgcgcaaaagggaccacccccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 93 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaagaaagcgacggatacacttttcttcactggttccaacaaagacccggacaaagcccacgcctgcttatttacgaggtatcaaccagattctcaggagttccagacagattttcaggcagcggatccggcacagacttcacccttaaaattagcagagtagaagcagaagatgtaggagtgtattattgtttccaagccacccacgaccccctgaccttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 94 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcaaccaggtggatctctcagactttcatgtgccgcaagcggttttacttttagtaactatggaatgaactgggttagacaagcgcccggaaaaggattggaatggataggcatgatatattacgacagctcggagaaacattatgccgactcagttaaaggaagatttacaatatcaagagacaatagcaaaaacacactttatcttcaaatgaattcactgcgagccgaggatacagcagtctattattgcgcaaaagggaccacccccgattactggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggaccaactcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgctgcatgaggctctgcacaaccactacacgcagaagagcctctccctgtctccgggtaaatga

SEQ ID NO: 95 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgcggacagcgacggagatacttttcttcactggttccaacaaagacccggacaaagcccacgcctgcttatttacgcggtatcacacagattctcaggagttccagacagattttcaggcagcggatccggcacagacttcacccttaaaattagcagagtagaagcagaagatgtaggagtgtattattgtttccaagccacccatgaccccgtcacgttcggacaaggaactaagctcgaaatcaaa

SEQ ID NO: 96 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgcggacagcgacggagatacttttcttcactggttccaacaaagacccggacaaagcccacgcctgcttatttacgcggtatcacacagattctcaggagttccagacagattttcaggcagcggatccggcacagacttcacccttaaaattagcagagtagaagcagaagatgtaggagtgtattattgtttccaagccacccatgaccccgtcacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 97 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggcatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggcacgacgcccgataaatggggtcaaggaacaatggtaaccgtgtcaagc

SEQ ID NO: 98 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggagagcgacggatacacttttcttgagtggttccaacaaagacccggacaaagcccacgcctgcttatttacgaggtatcacatagattctcaggagttccagacagattttcaggcagcggatccggcacagacttcacccttaaaattagcagagtagaagcagaagatgtaggagtgtattattgtttccaagcgacccacgaccccctgacgttcggacaaggaactaagctcgaaatcaaa

SEQ ID NO: 99 - DNA, 1338 bp, synthetic construct
  misc_feature 1..1338: note = Synthethic Polynucleotide
  source 1..1338: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggcatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggcacgacgcccgataaatggggtcaaggaacaatggtaaccgtgtcaagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgcgaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 100 - DNA, 660 bp, synthetic construct
  misc_feature 1..660: note = Synthethic Polynucleotide
  source 1..660: mol_type = other DNA
gacgtagtactgacccaaagccccctttctctcccagtaaccctcggacaaccagcctcaatttcatgcagatcatcacaatcacttgaggagagcgacggatacacttttcttgagtggttccaacaaagacccggacaaagcccacgcctgcttatttacgaggtatcacatagattctcaggagttccagacagattttcaggcagcggatccggcacagacttcacccttaaaattagcagagtagaagcagaagatgtaggagtgtattattgtttccaagcgacccacgaccccctgacgttcggacaaggaactaagctcgaaatcaaaagaacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 101 - DNA, 345 bp, synthetic construct
  misc_feature 1..345: note = Synthethic Polynucleotide
  source 1..345: mol_type = other DNA
gaagtacagttggtagaaagcggcggaggacttgtgcagccaggtggatctctcagactttcatgtgccgcaagcggttttacttttagtaactatggaatgaactgggttagacaagcgcccggaaaaggattggaatggataggaatgatatactacgacagctccgagaaacattatgccgactcagttaaaggaagatttacaatatcaagagacaatagcaaaaacacactttatcttcaaatgaattcactgcgagccgaggatacagcagtctattattgcgcaaaaggctcgacccccgattactggggtcaaggaacaatggtaaccgtgtcaagc

SEQ ID NO: 102 - DNA, 336 bp, synthetic construct
  misc_feature 1..336: note = Synthethic Polynucleotide
  source 1..336: mol_type = other DNA
gacgtagtactgacccaaagccccctttctctcccagtaaccctcggacaaccagcctcaatttcatgcagatcatcacaatcacttgaggacagcgacggaggcacttttcttgagtggttccaacaaagacccggacaaagcccacgcctgcttatttacgacgtatcaagcagattctcaggagttccagacagattttcaggcagcggatccggcacagacttcacccttaaaattagcagagtagaagcagaagatgtaggagtgtattattgtttccaagccacccacgaccccctcagcttcggacaaggaactaagctcgaaatcaaa

SEQ ID NO: 103 - protein, 330 aa, Homo sapiens [sequence omitted]

SEQ ID NO: 104 - protein, 19 aa, synthetic construct
  REGION 1..19: note = Synthethic Polypeptide
MEFGLSWLFLVAILKGVQC

SEQ ID NO: 105 - protein, 5 aa, synthetic construct
  REGION 1..5: note = Synthethic Polypeptide
  VARIANT 1: note = X can be N or Y
XYGMN

SEQ ID NO: 106 - protein, 17 aa, synthetic construct
  REGION 1..17: note = Synthethic Polypeptide
  VARIANT 8: note = X can be E or D
MIYYDSSXKHYADSVKG

SEQ ID NO: 107 - protein, 6 aa, synthetic construct
  REGION 1..6: note = Synthethic Polypeptide
  VARIANT 2: note = X can be T or S
  VARIANT 6: note = X can be Y, V, or K
GXTPDX

SEQ ID NO: 108 - protein, 16 aa, synthetic construct
  REGION 1..16: note = Synthethic Polypeptide
  VARIANT 7: note = X can be A or E
  VARIANT 8: note = X can be T, S, E, or D
  VARIANT 12: note = X can be D, Y, or G
  VARIANT 16: note = X can be E or H
RSSQSLXXSDGXTFLX

SEQ ID NO: 109 - protein, 7 aa, synthetic construct
  REGION 1..7: note = Synthethic Polypeptide
  VARIANT 1: note = X can be E, D, or A
  VARIANT 4: note = X can N, S, T, E, or H
XVSXRFS

SEQ ID NO: 110 - protein, 9 aa, synthetic construct
  REGION 1..9: note = Synthethic Polypeptide
  VARIANT 1: note = X can be F or M
  VARIANT 3: note = X can be V or A
  VARIANT 5: note = X can be H or Y
  VARIANT 8: note = X can be M, L, or V
  VARIANT 9: note = X can be T or S
XQXTXDPXX

SEQ ID NO: 111 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 112 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 113 - protein, 329 aa, synthetic construct
  REGION 1..329: note = Synthetic Polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 114 - protein, 444 aa, synthetic construct
  REGION 1..444: note = Synthetic Polypeptide
EVQLVESGGGLVQPGGSLRLSCAASGFTFSNYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 115 - protein, 444 aa, synthetic construct
  REGION 1..444: note = Synthetic Polypeptide
EVQLVESGGGLVQPGGSLRLSCAASGFTFSYYGMNWIRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 116 - protein, 444 aa, synthetic construct
  REGION 1..444: note = Synthetic Polypeptide
EVQLVESGGGLVQPGGSLRLSCAASGFTFSNYGMNWIRQAPGKGLEWIGMIYYDSSDKHYADSVKGRFTISRDNSKNTLYLQMNSLRAEDTAVYYCAKGTTPDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 117 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 118 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDQLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHNHYTQKSLSLSPG

SEQ ID NO: 119 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGTTPDKWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 120 - protein, 444 aa, synthetic construct
  REGION 1..444: note = Synthetic Polypeptide
EVQLVESGGGLVQPGGSLRLSCAASGFTFSNYGMNWVRQAPGKGLEWIGMIYYDSSEKHYADSVKGRFTISRDNSKNTLYLQMNSLRAEDTAVYYCAKGSTPDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 121 - protein, 14 aa, synthetic construct
  REGION 1..14: note = Synthetic
SSPMALGANATATR

SEQ ID NO: 122 - protein, 14 aa, synthetic construct
  REGION 1..14: note = Synthetic
TSSPMALGANATAT

SEQ ID NO: 123 - protein, 364 aa, synthetic construct
  REGION 1..364: note = Synthetic
QCKILRCNAEYVSSTLSLRGGGSSGALRGGGGGGRGGGVGSGGLCRALRSYALCTRRTARTCRGDLAFHSAVHGIEDLMIQHNCSRQGPTPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSD

SEQ ID NO: 124 - protein, 21 aa, synthetic construct
  REGION 1..21: note = Synthetic
SSPMALGANATATRKLTIIFK

SEQ ID NO: 125 - protein, 30 aa, synthetic construct
  REGION 1..30: note = Synthetic
DNDFLFVQATSSPMALGANATTRKLTIIFK

SEQ ID NO: 126 - protein, 21 aa, synthetic construct
  REGION 1..21: note = Synthetic
QLCVGGCPPSQRLSRSERNRR

SEQ ID NO: 127 - protein, 12 aa, synthetic construct
  REGION 1..12: note = Synthetic
SQRLSRSERNRR

SEQ ID NO: 128 - protein, 426 aa, Homo sapiens
MGEPGQSPSPRSSHGSPPTLSTLTLLLLLCGHAHSQCKILRCNAEYVSSTLSLRGGGSSGALRGGGGGGRGGGVGSGGLCRALRSYALCTRRTARTCRGDLAFHSAVHGIEDLMIQHNCSRQGPTAPPPPRGPALPGAGSGLPAPDPCDYEGRFSRLHGRPPGFLHCASFGDPHVRSFHHHFHTCRVQGAWPLLDNDFLFVQATSSPMALGANATATRKLTIIFKNMQECIDQKVYQAEVDNLPVAFEDGSINGGDRPGGSSLSIQTANPGNHVEIQAAYIGTTIIIRQTAGQLSFSIKVAEDVAMAFSAEQDLQLCVGGCPPSQRLSRSERNRRGAITIDTARRLCKEGLPVEDAYFHSCVFDVLISGDPNFTVAAQAALEDARAFLPDLEKLHLFPSDAGVPLSSATLLAPLLSGLFVLWLCIQ

SEQ ID NO: 129 - protein, 25 aa, synthetic construct
  REGION 1..25: note = Synthetic
DTHFPICIFCCGCCHRSKCGMCCKT